SEQUENCE LISTING (35 sequences)

SEQ ID NO: 1 - DNA, 2939 bp, Artificial Sequence
  PLE-wu transposon sequence
ttaacccttttgcagttagagggaatatttttcccaccaataaaaaccaataaaaaaggaacaaaatttctatttgcaattttatttttcgacctgacaacattaacgtaacattatgacatttgtttctgattttatttgacattgacagattcaattttttttcaaagtcttgaggtgaatttgaaaactagcggttttttgtacttcgagagaaatcggtacatatctttgcaatagagtggtgaaaaaaggtaagttcttctagatgttgtgcttttgaatgttttttcatagatataagacgtattttttatgtatctgccacgaattttcatatatgtcgattttttttggtgggaagaatattccctctaaccgtgaagtatccgctttctactaatacgtaaggttagagaaaatattcctttattatgtttacggtacctgtttttattgcttgcggctttttctcggggcttggtgtggtttttgcatggcttttgtttttttatttgcagattgagtgaaaatgaaaatacttagagatgacgatattgagaaggagttgaatggtatgttcggtttgtcagaaccggaagagtctgaagacggcgaagaatcagaagacgatgacgttccgtacaatgtcactaagttacagcgaattttaggggatttagaagaaccagaccttcgatctactgcaacaattcgagcatcgttatcgcctcaactatcaacatcatctgatgcggagatcccatgcggtgagcctttacttgtaatacctccacctactgtgatagaagctgttgcgtctacgtctgcagtgccattacatgtgccgcaaccagctgtactactgcctgctgagcctcctatttcacgtaagttgcctgtaaaaggtagaggacgaggagctaggatggaacgtggatccggaacgggacgtagatttagaacaggacgagaagctggaactagaatgggacgtggagctagaacgaggctaggaagtggggttggagataaagacagcgattcatccgaagataatacgccaccagtatcagaagcagagtcagaagcaagcgattctgacagagatgaagaagagtggaaaaaaaccttgtggactgatattaggccacagcttgagtcctttgatagtgttccaatgactccgacacgcgtgttaccatctaatgccaggcctatccgatattttgaaaaattttttactcaagaagtttttgaattaattatcacagagactaacagatatgcatgtcaaaacaacgtaataggctggaccatactagacataaaggaattgaaggctttcctaggtatattgattattatgggatacaatattttacctactttcgaattgtattggtcatcggatcccgaatttagagttgacgaaatagcttccaccatgacattccgaagattcaagcaaattttacgttgtttgcatctaaatgataattctaaacaacctgccagacttagccccgagtatgataagctattcaaaattcgcccgttattgacgcttataaacacctcttttcaagaaaatgcgcacaactcttcctctcagtcaatagatgaatcaatgattcttttcaaaggaaggtctacattgaagcaatacatgcctatgaagccaataaaaaggggtttcaaggtatggtgccgctgtgatagtataactggttatctgtacgaatttgatatatacaccggaagagatggtgacagagtagaagacaatttaggaggaaaagtggtcaaaaagttaacagaaaaattgaaaggaatggcggcagtgcatgttacatttgataattttttttgcagttatgatatcatgaattatctacatgtaaatggtatttctgcgagcggtactgtacgcaggcaaagagcagacttaccgaaacttgtgaaaagtaaaaaaaaactaaaactaacaaaagggcagtacaaatggagagtgaaggagaacgtggcttttgtaatctggcaagatacaaaagaagtcttatttatgaccaatgctttccatcccaaggacaatgaaacatctctacccaggaaaggaagagacggttccaagacagatgtaaggtgtcctgcagtagtgaaagaatatacaaaaagaatgggtggagtagatcactttgatcacatcaaagggacatactcagtggggcgaaggagcaaacggtggtggcttcgcatattttattttatttttgatgcctgcatcacaaattcttttttactccagggaaagaatgcgaatgcaaccaaattgtccaacctagagtatcgtgtcgctctagccagaggacttattggttgtttttcttcccgaaaacgtcgtgctgaaggcgtcaattacgtcgtaagaaaaaaagttgctgtatctgaaaattatcaaaaggcaattcacatcgttgcaccggaaattcgcttttccaacgttggtgatcacatgccaaatgatataccgtcttatcaaagatgccgatattgcagcactaaggcaaaagataagcgatctaagatcaagtgtagcaaatgtggagtaccgttatgtataacaccatgcttttcgaattttcacaaacaagtttaattttcttttatttttgtttctattttcttattggtattcaattgtgtgattgaaataaagttattatgtgagatttatgttgtttttttagtttgttgaaaaatcatactacaatttcattactctcaaacaggtagagggaatattcttaccaccaattaaaaaaaaaagtttaaaaaaaattgaatctcaatttttattttaggcatcttatgtaacacaaatccgaaaaccaaatctttctatctcatctagatccttataaccgttaaagggttaa

SEQ ID NO: 2 - protein, 708 aa, Artificial sequence
  PLE-wu transposase amino acid sequence
MKILRDDDIEKELNGMFGLSEPEESEDGEESEDDDVPYNVTKLQRILGDLEEPDLRSTATIRASLSPQLSTSSDAEIPCGEPLLVIPPPTVIEAVASTSAVPLHVPQPAVLLPAEPPISRKLPVKGRGRGARMERGSGTGRRFRTGREAGTRMGRGARTRLGSGVGDKDSDSSEDNTPPVSEAESEASDSDRDEEEWKKTLWTDIRPQLESFDSVPMTPTRVLPSNARPIRYFEKFFTQEVFELIITETNRYACQNNVIGWTILDIKELKAFLGILIIMGYNILPTFELYWSSDPEFRVDEIASTMTFRRFKQILRCLHLNDNSKQPARLSPEYDKLFKIRPLLTLINTSFQENAHNSSSQSIDESMILFKGRSTLKQYMPMKPIKRGFKVWCRCDSITGYLYEFDIYTGRDGDRVEDNLGGKVVKKLTEKLKGMAAVHVTFDNFFCSYDIMNYLHVNGISASGTVRRQRADLPKLVKSKKKLKLTKGQYKWRVKENVAFVIWQDTKEVLFMTNAFHPKDNETSLPRKGRDGSKTDVRCPAVVKEYTKRMGGVDHFDHIKGTYSVGRRSKRWWLRIFYFIFDACITNSFLLQGKNANATKLSNLEYRVALARGLIGCFSSRKRRAEGVNYVVRKKVAVSENYQKAIHIVAPEIRFSNVGDHMPNDIPSYQRCRYCSTKAKDKRSKIKCSKCGVPLCITPCFSNFHKQV

SEQ ID NO: 3 - DNA, 2166 bp, Artificial sequence
  PLE-wu transposase nucleotide sequence
atgaaaatacttagagatgacgatattgagaaggagttgaatggtatgttcggtttgtcagaaccggaagagtctgaagacggcgaagaatcagaagacgatgacgttccgtacaatgtcactaagttacagcgaattttaggggatttagaagaaccagaccttcgatctactgcaacaattcgagcatcgttatcgcctcaactatcaacatcatctgatgcggagatcccatgcggtgagcctttacttgtaatacctccacctactgtgatagaagctgttgcgtctacgtctgcagtgccattacatgtgccgcaaccagctgtactactgcctgctgagcctcctatttcacgtaagttgcctgtaaaaggtagaggacgaggagctaggatggaacgtggatccggaacgggacgtagatttagaacaggacgagaagctggaactagaatgggacgtggagctagaacgaggctaggaagtggggttggagataaagacagcgattcatccgaagataatacgccaccagtatcagaagcagagtcagaagcaagcgattctgacagagatgaagaagagtggaaaaaaaccttgtggactgatattaggccacagcttgagtcctttgatagtgttccaatgactccgacacgcgtgttaccatctaatgccaggcctatccgatattttgaaaaattttttactcaagaagtttttgaattaattatcacagagactaacagatatgcatgtcaaaacaacgtaataggctggaccatactagacataaaggaattgaaggctttcctaggtatattgattattatgggatacaatattttacctactttcgaattgtattggtcatcggatcccgaatttagagttgacgaaatagcttccaccatgacattccgaagattcaagcaaattttacgttgtttgcatctaaatgataattctaaacaacctgccagacttagccccgagtatgataagctattcaaaattcgcccgttattgacgcttataaacacctcttttcaagaaaatgcgcacaactcttcctctcagtcaatagatgaatcaatgattcttttcaaaggaaggtctacattgaagcaatacatgcctatgaagccaataaaaaggggtttcaaggtatggtgccgctgtgatagtataactggttatctgtacgaatttgatatatacaccggaagagatggtgacagagtagaagacaatttaggaggaaaagtggtcaaaaagttaacagaaaaattgaaaggaatggcggcagtgcatgttacatttgataattttttttgcagttatgatatcatgaattatctacatgtaaatggtatttctgcgagcggtactgtacgcaggcaaagagcagacttaccgaaacttgtgaaaagtaaaaaaaaactaaaactaacaaaagggcagtacaaatggagagtgaaggagaacgtggcttttgtaatctggcaagatacaaaagaagtcttatttatgaccaatgctttccatcccaaggacaatgaaacatctctacccaggaaaggaagagacggttccaagacagatgtaaggtgtcctgcagtagtgaaagaatatacaaaaagaatgggtggagtagatcactttgatcacatcaaagggacatactcagtggggcgaaggagcaaacggtggtggcttcgcatattttattttatttttgatgcctgcatcacaaattcttttttactccagggaaagaatgcgaatgcaaccaaattgtccaacctagagtatcgtgtcgctctagccagaggacttattggttgtttttcttcccgaaaacgtcgtgctgaaggcgtcaattacgtcgtaagaaaaaaagttgctgtatctgaaaattatcaaaaggcaattcacatcgttgcaccggaaattcgcttttccaacgttggtgatcacatgccaaatgatataccgtcttatcaaagatgccgatattgcagcactaaggcaaaagataagcgatctaagatcaagtgtagcaaatgtggagtaccgttatgtataacaccatgcttttcgaattttcacaaacaagtttaattttcttttatttttgtttctattttcttattggtattc

SEQ ID NO: 4 - DNA, 379 bp, Artificial sequence
  PLE-wu 5′ end
ttaacccttttgcagttagagggaatatttttcccaccaataaaaaccaataaaaaaggaacaaaatttctatttgcaattttatttttcgacctgacaacattaacgtaacattatgacatttgtttctgattttatttgacattgacagattcaattttttttcaaagtcttgaggtgaatttgaaaactagcggttttttgtacttcgagagaaatcggtacatatctttgcaatagagtggtgaaaaaaggtaagttcttctagatgttgtgcttttgaatgttttttcatagatataagacgtattttttatgtatctgccacgaattttcatatatgtcgattttttttggtgggaagaatattccctctaac

SEQ ID NO: 5 - DNA, 148 bp, Artificial Sequence
  PLE-wu 3′ end
ggtagagggaatattcttaccaccaattaaaaaaaaaagtttaaaaaaaattgaatctcaatttttattttaggcatcttatgtaacacaaatccgaaaaccaaatctttctatctcatctagatccttataaccgttaaagggttaa

SEQ ID NO: 6 - DNA, 27 bp, Artificial sequence
  Forward primer for amplifying PLE-wu transposon
gcggaattcgcaacagagcgtcgcgag

SEQ ID NO: 7 - DNA, 29 bp, Artificial sequence
  Reverse primer for amplifying PLE-wu transposon
gcggaattcagcggagcaaaagctgttac

SEQ ID NO: 8 - DNA, 37 bp, Artificial sequence
  Forward primer for amplifying 708-residue PLE-wu transposase
cgcgaattcgccaccatgaaaatacttagagatgacg

SEQ ID NO: 9 - DNA, 37 bp, Artificial sequence; identical to SEQ ID NO: 8
  Reverse primer for amplifying 708-residue PLE-wu transposase

SEQ ID NO: 10 - DNA, 29 bp, Artificial sequence
  Forward primer for amplifying Full-5′ end of PLE-wu
gctgaattcaacacgcgactcttttcgtc

SEQ ID NO: 11 - DNA, 27 bp, Artificial sequence
  Reverse primer for amplifying Full-5′ end of PLE-wu (1)
gcgactagtagaaagcggatacttcac

SEQ ID NO: 12 - DNA, 32 bp, Artificial sequence
  Forward primer for amplifying Full-3′ end of PLE-wu
gcgacgcgtattggtattcaattgtgtgattg

SEQ ID NO: 13 - DNA, 29 bp, Artificial sequence
  Reverse primer for amplifying Full-3′ end of PLE-wu
gctgaattcaagaatggcaaaccaagtcg

SEQ ID NO: 14 - DNA, 29 bp, Artificial sequence
  Forward primer for checking for excision of PLE-wu (P1)
cgcctcgaggacgtctggcaagaatcaca

SEQ ID NO: 15 - DNA, 29 bp, Artificial sequence
  Reverse primer for checking for excision of PLE-wu (P2)
cgcaagcttcacgacagcaggctgaataa

SEQ ID NO: 16 - DNA, 20 bp, Artificial sequence
  Forward primer for checking for excision of PLE-wu (P3)
gaaccgtcagatccgctagt

SEQ ID NO: 17 - DNA, 20 bp, Artificial sequence
  Reverse primer for checking for excision of PLE-wu (P4)
gaacttcagggtcagcttgc

SEQ ID NO: 18 - DNA, 35 bp, Artificial sequence
  Forward primer for amplifying 576-residue transposase
cgcgaattcgccaccatggaacgtggatccggaac

SEQ ID NO: 19 - DNA, 32 bp, Artificial sequence
  Reverse primer for amplifying 576-residue transposase
gcggaattcttaaacttgtttgtgaaaattcg

SEQ ID NO: 20 - DNA, 30 bp, Artificial sequence
  Reverse primer for amplifying Full-5′ end of PLE-wu (2)
gcgacgcgtagtagaaagcggatacttcac

SEQ ID NO: 21 - DNA, 29 bp, Artificial sequence
  Forward primer for amplifying deleted-5′ end of PLE-wu (i.e. short PLE-wu/short Tpase)
gcgacgcgttacgttaatgttgtcaggtc

SEQ ID NO: 22 - DNA, 31 bp, Artificial sequence
  Forward primer for amplifying deleted-3′ end of PLE-wu (i.e. short PLE-wu/short Tpase)
gcgacgcgtgcatcttatgtaacacaaatcc

SEQ ID NO: 23 - DNA, 20 bp, Artificial sequence
  Reverse primer for amplifying 3′end of PLE-wu and its nearby chromosome region
tgaacccaccacatttcaga

SEQ ID NO: 24 - DNA, 112 bp, Artificial sequence
  5′ end of short/ truncated PLE-wu transposon
ttaacccttttgcagttagagggaatatttttcccaccaataaaaaccaataaaaaaggaacaaaatttctatttgcaattttatttttcgacctgacaacattaacgtaac

SEQ ID NO: 25 - DNA, 74 bp, Artificial sequence
  3′ end of truncated / short PLE-wu transposon
catcttatgtaacacaaatccgaaaaccaaatctttctatctcatctagatccttataaccgttaaagggttaa

SEQ ID NO: 26 - DNA, 21 bp, Artificial sequence
  Forward primer for amplifying 3′end of PLE-wu and its nearby chromosome region
cacaaatccgaaaaccaaatc

SEQ ID NO: 27 - protein, 576 aa, Artificial sequence
  PLE-wu short transposase
MERGSGTGRRFRTGREAGTRMGRGARTRLGSGVGDKDSDSSEDNTPPVSEAESEASDSDRDEEEWKKTLWTDIRPQLESFDSVPMTPTRVLPSNARPIRYFEKFFTQEVFELIITETNRYACQNNVIGWTILDIKELKAFLGILIIMGYNILPTFELYWSSDPEFRVDEIASTMTFRRFKQILRCLHLNDNSKQPARLSPEYDKLFKIRPLLTLINTSFQENAHNSSSQSIDESMILFKGRSTLKQYMPMKPIKRGFKVWCRCDSITGYLYEFDIYTGRDGDRVEDNLGGKVVKKLTEKLKGMAAVHVTFDNFFCSYDIMNYLHVNGISASGTVRRQRADLPKLVKSKKKLKLTKGQYKWRVKENVAFVIWQDTKEVLFMTNAFHPKDNETSLPRKGRDGSKTDVRCPAVVKEYTKRMGGVDHFDHIKGTYSVGRRSKRWWLRIFYFIFDACITNSFLLQGKNANATKLSNLEYRVALARGLIGCFSSRKRRAEGVNYVVRKKVAVSENYQKAIHIVAPEIRFSNVGDHMPNDIPSYQRCRYCSTKAKDKRSKIKCSKCGVPLCITPCFSNFHKQV

SEQ ID NO: 28 - DNA, 1731 bp, Artificial sequence
  PLE-wu nucleotide sequence encoding short transposase
atggaacgtggatccggaacgggacgtagatttagaacaggacgagaagctggaactagaatgggacgtggagctagaacgaggctaggaagtggggttggagataaagacagcgattcatccgaagataatacgccaccagtatcagaagcagagtcagaagcaagcgattctgacagagatgaagaagagtggaaaaaaaccttgtggactgatattaggccacagcttgagtcctttgatagtgttccaatgactccgacacgcgtgttaccatctaatgccaggcctatccgatattttgaaaaattttttactcaagaagtttttgaattaattatcacagagactaacagatatgcatgtcaaaacaacgtaataggctggaccatactagacataaaggaattgaaggctttcctaggtatattgattattatgggatacaatattttacctactttcgaattgtattggtcatcggatcccgaatttagagttgacgaaatagcttccaccatgacattccgaagattcaagcaaattttacgttgtttgcatctaaatgataattctaaacaacctgccagacttagccccgagtatgataagctattcaaaattcgcccgttattgacgcttataaacacctcttttcaagaaaatgcgcacaactcttcctctcagtcaatagatgaatcaatgattcttttcaaaggaaggtctacattgaagcaatacatgcctatgaagccaataaaaaggggtttcaaggtatggtgccgctgtgatagtataactggttatctgtacgaatttgatatatacaccggaagagatggtgacagagtagaagacaatttaggaggaaaagtggtcaaaaagttaacagaaaaattgaaaggaatggcggcagtgcatgttacatttgataattttttttgcagttatgatatcatgaattatctacatgtaaatggtatttctgcgagcggtactgtacgcaggcaaagagcagacttaccgaaacttgtgaaaagtaaaaaaaaactaaaactaacaaaagggcagtacaaatggagagtgaaggagaacgtggcttttgtaatctggcaagatacaaaagaagtcttatttatgaccaatgctttccatcccaaggacaatgaaacatctctacccaggaaaggaagagacggttccaagacagatgtaaggtgtcctgcagtagtgaaagaatatacaaaaagaatgggtggagtagatcactttgatcacatcaaagggacatactcagtggggcgaaggagcaaacggtggtggcttcgcatattttattttatttttgatgcctgcatcacaaattcttttttactccagggaaagaatgcgaatgcaaccaaattgtccaacctagagtatcgtgtcgctctagccagaggacttattggttgtttttcttcccgaaaacgtcgtgctgaaggcgtcaattacgtcgtaagaaaaaaagttgctgtatctgaaaattatcaaaaggcaattcacatcgttgcaccggaaattcgcttttccaacgttggtgatcacatgccaaatgatataccgtcttatcaaagatgccgatattgcagcactaaggcaaaagataagcgatctaagatcaagtgtagcaaatgtggagtaccgttatgtataacaccatgcttttcgaattttcacaaacaagtttaa

SEQ ID NO: 29 - DNA, 20 bp, Artificial sequence
  5′ end PLE-wu imperfect inverted repeat
cccttttgcagttagaggga

SEQ ID NO: 30 - DNA, 20 bp, Artificial sequence
  3′ end PLE-wu imperfect inverted repeat
tccttataaccgttaaaggg

SEQ ID NO: 31 - DNA, 6 bp, Artificial sequence
  5′ end of PLE-wu perfect inverted terminal repeat
cccttt

SEQ ID NO: 32 - DNA, 6 bp, Artificial sequence
  3′ end of PLE-wu perfect inverted terminal repeats
aaaggg

SEQ ID NO: 33 - DNA, 32 bp, Artificial sequence
  Sub-terminal repeat 1
gttagagggaatatttttcccaccaataaaaa

SEQ ID NO: 34 - DNA, 32 bp, Artificial sequence
  Sub-terminal repeat 2
ttttttttggtgggaagaatattccctctaac

SEQ ID NO: 35 - DNA, 32 bp, Artificial sequence
  Sub-terminal repeat 3
ggtagagggaatattcttaccaccaattaaaa

(19) United States
(12) Patent Application Publication
(10) Pub. No.: US 2014/0349321 A1
(43) Pub. Date: Nov. 27, 2014

(54) BINDING MOLECULES AGAINST DENGUE VIRUS AND USES THEREOF

(71) Applicant: Agency for Science, Technology and Research, Singapore (SG)

(72) Inventors: Katja Fink, Singapore (SG); Cheng-I Wang, Singapore (SG)

(73) Assignee: Agency for Science, Technology and Research, Singapore (SG)

(21) Appl. No.: 14/365,853

(22) PCT Filed: Dec. 17, 2012
(86) PCT No.: PCT/SG2012/000477

§ 371 Date: Jun. 16, 2014

(30) Foreign Application Priority Data
Dec. 16, 2011 (SG) 201109401-8

(51) Int. Cl. C07K 16/10; G01N 33/543

(52) U.S. Cl. CPC C07K 16/1081; G01N 33/54306; G01N 2333/47; C07K 2317/10
USPC 435/7.92; 530/388.15; 530/387.3; 536/23.53

(57) ABSTRACT

The present invention relates to binding molecules suited to the diagnosis, prevention and/or treatment of dengue virus infection.

### INCORPORATION BY CROSS-REFERENCE

This application claims priority from Singaporean Patent Application No. 201109401-8 filed on 16 Dec. 2011, the entire contents of which are incorporated herein by cross-reference.

### TECHNICAL FIELD

The present invention relates generally to the field of infectious diseases. More specifically, the present invention relates to the diagnosis and treatment of dengue virus infection.

### BACKGROUND

Dengue is a prevalent mosquito-borne flavivirus causing significant human disease ranging from dengue fever to life-threatening dengue hemorrhagic fever/dengue shock syndrome (DHF/DSS). It is estimated that 2.5 billion people are at risk of dengue virus infection with 50-100 million cases of dengue fever annually causing approximately 25,000 deaths (predominantly among children).

The symptoms of dengue disease are primarily immune-mediated, with the most severe manifestations generally occurring after patient viremia has decreased from its peak. Typical dengue symptoms include high fever, vascular leakage, rashes, headache and bone and muscle pain. Dengue patients thus exhibit hallmarks of severe inflammation including increased serum cytokine concentrations, high numbers of activated T and B cells, and high titers of circulating antibodies. While dengue fever is an acute, self-limiting illness, DHF/DSS is a plasma leakage syndrome characterised by defects in vascular permeability, marked thrombocytopenia, hepatomegaly and bleeding diathesis, which can lead to life-threatening shock. The rapid spread of dengue virus to most tropical and subtropical countries has led to its classification as an emerging infectious disease and has intensified efforts to prevent infection.

There are four serotypes of dengue virus and each can cause the full spectrum of disease. It is possible for a single individual to be infected four separate times by these alternative strains of virus. Intriguingly, successive rounds of infection seem to be associated with increased disease severity, suggesting that pre-existing immunity can have a detrimental effect on the course of disease.

Sera from infected patients has been found to contain antibodies against dengue virus structural proteins (including prM, the E glycoprotein, and capsid (C) protein), and also against non-structural proteins, (primarily against NS1, which is secreted by infected cells). In particular, the E glycoprotein which is exposed on the surface of the particle induces significant humoral immune responses. The E glycoprotein consists of three domains I, II and III. E domain III (EDIII) sticks out from the virus coat of infectious virus particles and is therefore easily accessible for antibodies. EDIII protein is required for viral attachment to host cells and blocking of EDIII protein by antibodies most efficiently neutralizes the virus. Antibodies blocking other regions of the E protein (EDI and EDII) can also inhibit infection, but the binding of the antibodies is usually of lower affinity and the inhibition less efficient. Unexpectedly, the immune response in patients seems to produce many antibodies that are EDII-specific, whereas EDIII-specific antibodies are more rare. Hence, host antibody responses against the E glycoprotein can be relatively ineffective.

While EDIII protein sequences are less conserved than EDII protein, many EDIII-specific antibodies are also serotype cross-reactive, since only specific loops in EDIII contain serotype-specific mutations. In in vitro neutralization assays both EDII- and EDIII-specific antibodies can be neutralizing and serotype-specific. Since only a small fraction of antibodies in the serum bind serotype-specific epitopes on EDIII protein, their effect is usually masked by a majority of cross-reactive antibodies. In vitro neutralization assays are thus often very difficult to interpret and cannot clearly identify the serotypes of previous infections.

Despite the prevalence and seriousness of dengue disease there are few if any effective preventative and treatment strategies available. Successive rounds of dengue infection are associated with increased disease severity. The degree and quality of immunity induced against specific dengue subtypes following vaccination and/or initial dengue infection is thus an important consideration in the design of therapeutic and preventative strategies (e.g. re-vaccination). However, it remains difficult to assess.

In dengue diagnostics, the neutralization assay is the gold standard to assess protective antibody titers. However, due to the inherent biophysical characteristics of dengue virus it is almost impossible to standardize the neutralization assay (Dowd et al. 2011; WHO, 2009). The neutralization assay therefore offers limited value and additional diagnostic tools are needed (Sabchareon et al. 2012). Furthermore, current treatments for dengue infection rely primarily on alleviating symptoms rather than assisting host immune responses against the virus.

A need therefore exists for improved dengue virus diagnostic tools, and agents which aid host immune responses against dengue virus infection.

### SUMMARY OF THE INVENTION

The present inventors have identified a series of binding molecules beneficial for the diagnosis, prevention and treatment of dungue virus infection.

The present invention relates at least to the following embodiments:

1. A human monoclonal antibody capable of specifically binding to an envelope (E) protein of at least one dengue virus serotype, wherein the antibody comprises a light chain variable domain sequence comprising the first, second, and third complementarity determining region (CDR) sequences of a single clone designated in FIG. 20, 21 or 22.

2. A cross-reactive human monoclonal antibody derived from a memory B lymphocyte capable of specifically binding to an E protein of at least one dengue virus serotype, wherein the antibody comprises all CDR sequences from one pair of the following heavy and light chain variable domain peptide sequences: SEQ ID NOs: 189 and 190; SEQ ID NOs: 191 and 192; SEQ ID NOs: 199 and 200; SEQ ID NOs: 201 and 202: SEQ ID NOs: 203 and 204; SEQ ID NOs: 207 and 208; SEQ ID NOs: 209 and 210; SEQ ID NOs: 211 and 212; SEQ ID NOs: 213 and 214; or SEQ ID NOs: 215 and 216.

3. A cross-reactive human monoclonal antibody derived from a plasmablast capable of specifically binding to an E protein of at least one dengue virus serotype, wherein the antibody comprises all CDR sequences from one pair of the following heavy and light chain variable domain peptide sequences: SEQ ID NOs: 113 and 114; SEQ ID NOs: 115 and 116; SEQ ID NOs: 121 and 122; SEQ ID NOs: 123 and 124; SEQ ID NOs: 127 and 128; SEQ ID NOs: 129 and 130; SEQ ID NOs: 131 and 132; SEQ ID NOs: 133 and 134; SEQ ID NOs: 135 and 136; SEQ ID NOs: 137 and 138; SEQ ID NOs: 139 and 140; SEQ ID NOs: 141 and 142; SEQ ID NOs: 143 and 144; SEQ ID NOs: 145 and 146; SEQ ID NOs: 147 and 148; SEQ ID NOs: 151 and 152; SEQ ID NOs: 153 and 154; SEQ ID NOs: 155 and 156; SEQ ID NOs: 157 and 158; SEQ ID NOs: 159 and 160; SEQ ID NOs: 161 and 162; SEQ ID NOs: 163 and 164; SEQ ID NOs: 165 and 166; SEQ ID NOs: 169 and 170; SEQ ID NOs: 171 and 172; SEQ ID NOs: 173 and 174; SEQ ID NOs: 177 and 178; SEQ ID NOs: 179 and 180; SEQ ID NOs: 181 and 182; SEQ ID NOs: 183 and 184; SEQ ID NOs: 187 and 188; SEQ ID NOs: 189 and 190; SEQ ID NOs: 191 and 192; SEQ ID NOs: 193 and 104: SEQ ID NOs: 195 and 196; SEQ ID NOs: 197 and 198; SEQ ID NOs: 199 and 200; SEQ ID NOs: 201 and 202; SEQ ID NOs: 203 and 204; SEQ ID NOs: 205 and 206; SEQ ID NOs: 207 and 208; SEQ ID NOs: 209 and 210; SEQ ID NOs: 211 and 212; SEQ ID NOs: 213 and 214; or SEQ ID NOs: 215 and 216.

4. A serotype-specific human monoclonal antibody derived from a plasmablast capable of specifically binding to an E protein of one or two dengue virus serotypes with higher affinity compared to E proteins of other dengue virus serotypes, wherein the antibody comprises all CDR sequences from one pair of the following heavy and light chain variable domain peptide sequences: SEQ ID NO: 109 and SEQ ID NO: 110; SEQ ID NO: 111 and SEQ ID: NO 112; SEQ ID NO: 119 and SEQ ID NO: 120: SEQ ID NO: 125 and SEQ ID NO: 126; SEQ ID NO: 149 and SEQ ID NO: 150; SEQ ID NO: 153 and SEQ ID NO: 154; SEQ ID NO: 167 and SEQ ID NO: 168; SEQ ID NO: 175 and SEQ ID NO: 176; SEQ ID NO: 0.185 or SEQ ID NO: 186.

5. A serotype-specific human monoclonal antibody derived from a memory B lymphocyte capable of specifically binding to an E protein of one or two dengue virus serotypes with higher affinity compared to E proteins of other dengue virus serotypes, wherein the antibody comprises all CDR sequences from one pair of the following heavy and light chain variable domain peptide sequences: SEQ ID NO: 193 and SEQ ID NO: 194; SEQ ID NO: 195 and SEQ ID NO: 196; SEQ ID NO: 197 or 198; or SEQ ID NO: 205 and SEQ ID NO: 206

6. The monoclonal antibody of any one of embodiments 1 to 5, wherein the antibody comprises full heavy and light chain variable domain sequences from said pair.

7. A bi-specific antibody comprising a variable domain of a first monoclonal antibody linked to a variable domain of a second monoclonal antibody, wherein the first and second antibody each comprise all CDR sequences from one pair of heavy and light chain variable domain peptide sequences according to any one of embodiments 1 to 6.

8. A bispecific antibody comprising a variable domain of a first monoclonal antibody linked to a variable domain of a second monoclonal antibody, wherein first and second antibodies are a combination as defined in Table 5 or Table 6.

9. A pharmaceutical composition or kit comprising the antibody of any one of embodiments 1 to 8.

10. An isolated polynucleotide encoding first, second, and third complementarity determining region (CDR) sequences in the heavy chain variable domain of a single clone designated in FIG. 20, 21 or 22.

11. An isolated polynucleotide encoding first, second, and third complementarity determining region (CDR) sequences in the light chain heavy chain variable domain of a single clone designated in FIG. 20, 21 or 22.

12. An isolated polynucleotide encoding an antibody according to any one of embodiments 1 to 8.

13. Use of the antibody according to any one of embodiments 1 to 8 for prevention, treatment or diagnosis of dengue virus infection.

14. Use of the antibody according to any one of embodiments 1 to 8 in the preparation of a medicament for prevention or treatment of dengue virus infection.

15. The use according to embodiment 13 or embodiment 14, wherein the antibody comprises:

all CDR sequences from any one or more of the following pairs of heavy and light chain variable domain peptide sequences (SEQ ID NOs: 109 and 110; SEQ ID NOs: 111 and 112; SEQ ID NOs: 117 and 118; SEQ ID NOs: 119 and 120; SEQ ID NOs: 125 and 126; SEQ ID NOs: 149 and 150; SEQ ID NOs: 153 and 154; SEQ ID NOs: 167 and 168; SEQ ID NOs: 175 and 176; SEQ ID NOs: 185 and 186; SEQ ID NOs: 189 and 190; SEQ ID NOs: 191 and 192; SEQ ID NOs: 193 and 194; SEQ ID NOs: 195 and 196; SEQ ID NOs: 197 and 198; SEQ ID NOs: 199 and 200; SEQ ID NOs: 201 and 202: SEQ ID NOs: 203 and 204; SEQ ID NOs: 205 and 206; SEQ ID NOs: 207 and 208; SEQ ID NOs: 209 and 210; SEQ ID NOs: 211 and 212; SEQ ID NOs: 213 and 214; SEQ ID NOs: 215 and 216; or

a series of heavy and light chain variable domain CDR sequences in accordance with any one clone as designated in FIG. 22 herein.

16. An antibody according to any one of embodiments 1 to 8 for use in the prevention or treatment of dengue virus infection.

17. The antibody according to embodiment 16, wherein the antibody comprises:

all CDR sequences from any one or more of the following pairs of heavy and light chain variable domain peptide sequences (SEQ ID NOs: 109 and 110; SEQ ID NOs: 111 and 112; SEQ ID NOs: 117 and 118; SEQ ID NOs: 119 and 120; SEQ ID NOs: 125 and 126; SEQ ID NOs: 149 and 150; SEQ ID NOs: 153 and 154; SEQ ID NOs: 167 and 168; SEQ ID NOs: 175 and 176; SEQ ID NOs: 185 and 186; SEQ ID NOs: 189 and 190; SEQ ID NOs: 191 and 192; SEQ ID NOs: 193 and 194; SEQ ID NOs: 195 and 196; SEQ ID NOs: 197 and 198; SEQ ID NOs: 199 and 200; SEQ ID NOs: 201 and 202: SEQ ID NOs: 203 and 204; SEQ ID NOs: 205 and 206; SEQ ID NOs: 207 and 208; SEQ ID NOs: 209 and 210; SEQ ID NOs: 211 and 212; SEQ ID NOs: 213 and 214; SEQ ID NOs: 215 and 216; or

a series of heavy and light chain variable domain CDR sequences in accordance with any one clone as designated in FIG. 22 herein.

18. The use according to any one of embodiments 13 to 15, or the antibody according to claim 16 or claim 17, wherein said antibody comprises full heavy and light chain variable domain sequences from said pair

19. A method for detecting immunity to dengue virus in a subject, comprising:
coating at least one insoluble support with a dengue virus E protein,
contacting the E protein coated to the support with a first antibody comprising:
an antibody according to any one of embodiments 1 to 8; and
the biological sample of the subject;
and detecting a presence or absence of competitive binding to the E protein between the first antibody and an antibody specific to the E protein that may be present in the sample,
wherein detection of said competitive binding indicates pre-existing immunity to dengue virus in the subject.

20. The method according to embodiment 19, comprising:
coating four of said insoluble supports with E protein, wherein each said support is coated with E protein from a distinct dengue virus serotype, and each said support is isolated from all other said supports;
contacting the E protein coated to each said isolated insoluble support with said first antibody and said biological sample; and
determining the amount of first antibody bound to the E protein coated on each said isolated insoluble support;
wherein detection of less first antibody bound to the E protein coated on one of said supports when compared to at least one other of said supports indicates pre-existing immunity to dengue virus in the subject,
and wherein said pre-existing immunity is specific to the dengue serotype of the E protein to which less first antibody is bound.

21. The method according to embodiment 20, comprising:
coating four of said insoluble supports with E protein, wherein each said support is coated with E protein from the same dengue virus serotype, and each said support is isolated from all other said supports;
contacting the E protein coated to each said isolated insoluble support with said first antibody, said biological sample and soluble E protein of a specific dengue virus serotype,
wherein each said isolated insoluble support is contacted with soluble E protein from a different dengue virus serotype; and
determining the amount of first antibody bound to the E protein of each said isolated insoluble support;
wherein detection of less first antibody bound to the E protein coated on one of said supports when compared to at least one other of said supports indicates pre-existing immunity to dengue virus in the subject,
and wherein said pre-existing immunity is specific to the dengue serotype of the soluble E protein contacted with the E protein to which less first antibody is bound.

22. A method for detecting immunity to dengue virus in a subject, comprising:
coating four insoluble supports with E protein, wherein each said support is coated with E protein from a distinct dengue virus serotype, and each said support is isolated from all other said supports;
contacting the E protein coated on each said isolated support with a biological sample from the subject, and, a first antibody according to any one of claims 1 to 8;
wherein the first antibody contacted with the E protein coated on each said isolated insoluble support binds specifically to that said E protein, and cannot bind to an E protein coated to any other of said supports;
determining the amount of first antibody bound to the E protein of each said isolated insoluble support;
wherein detection of less first antibody bound to the E protein coated on one of said supports when compared to at least one other of said supports indicates pre-existing immunity to dengue virus in the subject,
and wherein said pre-existing immunity is specific to the dengue serotype of the E protein to which less first antibody is bound.

23. The method according to embodiment 22, wherein said first antibody is an antibody according to claim 4.

24. The method according to any one of embodiments 19 to 23, wherein any one or more of said insoluble supports is a bead or a well in culture plate.

25. The method according to any one of embodiments 19 to 24, wherein the first antibody is labelled with a detectable marker.

26. A method for detecting immunity to dengue virus in a subject, comprising:
coating at least one insoluble support with a first antibody comprising all heavy and light chain variable domain CDR sequences from an antibody according to any one of embodiments 1 to 8;
contacting the first antibody coated on the support with dengue virus particles to thereby allow said virus particles to bind to said first antibody;
contacting the virus particles with a biological sample from the subject to thereby allow any dengue virus-specific antibodies that may be present in said sample to bind to said virus particles; and
detecting whether said virus particles are bound by any said dengue virus-specific antibodies to thereby determine whether immunity to dengue virus exists in the subject.

27. The method according to embodiment 26, wherein the first antibody is only capable of binding to an E protein from one dengue virus serotype.

28. The method according to any one of embodiments 19 to 25, or embodiment 27, wherein the E protein is E domain III (EDIII) protein.

### BRIEF DESCRIPTION OF THE DRAWINGS

Preferred embodiments of the present invention will now be described, by way of example only, with reference to the accompanying figures wherein: FIG. 1 shows cross-reactive plasmablasts after secondary dengue infection. A) Experimental setup: individual plasmablasts were sorted into 96 well plates for Ig variable gene sequencing, cloning and expression of antibodies. B) ELISA to test DENV-specificity of recombinantly expressed mAbs. For each mAb clone (x axes), the OD450 values for DENV1, 2, 3 and 4 are represented by an individual data bar. Serum pooled from several dengue-immune individuals was used as a positive control. C) Representative clone 1B-H1L1 is shown for immunohistology with DENV-infected BHK-21 cells. Serum pooled from several dengue-immune individuals was used as a positive control and chikungunya-specific 5C-RF was used as a negative control. Clones showing a staining intensity and frequency comparable to the positive control were considered positive. D) 77% of the mAbs derived from patient 10/63 bound to DENV1, 2, 3, or 4 (as assessed by ELISA and/or histology). Cells were sorted at day 4 after onset of fever. Similarly, 60% of mAbs derived from patient 10/50 bound to DENV1, 2, 3, or 4 (as assessed by ELISA and/or histology). Cells were sorted at day 6 after onset of fever.

FIG. 2 PB-derived mAbs are cross-neutralizing in vitro but show serotype-specific protection in vivo. A) Neutralizing capacity of plasma from patients 10/63 and 10/50 against the serotype of the current infection at different time points postinfection. B) The neutralizing capacity of PB-derived mAbs from patients 10/63 and 10/50 was tested against all four DENV serotypes; the NT50 (means 6SD) of all tested mAbs is shown (10/63: n=12, 10/50: n=13). Patient 10/63 mAbs neutralize DENV2 and DENV3 significantly better than DENV1 and DENV4, as indicated by a lower mean NT50 value. The highest concentration tested was 10 mg/ml, and nonneutralizing Abs were arbitrarily assigned an NT50 of 10 (see Supplemental Table I for individual values). **p<0.005, ***p<0.0005, Kruskal-Wallis test with a Dunn multiple-comparison test. C) Neutralization for the clones that were tester later in vivo (in D) are shown in detail; neutralization of the respective serotypes of the previous and current infection for both patients is illustrated; 100% infection is equivalent to no neutralization. D) A total of 100 mg of the DENV-neutralizing mAb clones shown in B), derived from patient 10/63 and 10/50, and control mAb HA4 or 5F-RC were injected i.p. into AG129 mice 5-24 h before infection with DENV1, DENV2, or DENV3 (serotypes of the previous and current infection). DENV titers in the blood of mice were measured at day 3 postinfection, which corresponds with the peak of viremia. Each dot represents one mouse, and the mean value/group is indicated. The experiments were repeated with similar results. *p<0.05, **p<0.005, anti-DENV mAbs versus control mAb, one-way ANOVA with the Tukey multiple-comparison test.

FIG. 3 demonstrates that the plasmablast response is predominantly E protein specific. A) Twelve and fourteen clones from patient 10/63 and 10/50, respectively, were tested in ELISA for their binding to recombinant soluble E protein. Pie-charts show clones specific to E protein of one or more serotypes and radar diagrams show binding to the current and previous serotype of infection. B) For Western Blot, virus particles produced in C6/36 cells were lysed and proteins separated by SDS-PAGE under non-reducing conditions. Controls were pooled plasma from healthy donors with dengue immunity, EDIII-specific mAb 9F12, and negative control mAb HA4. With the exception of 9B-H1L1, all human mAbs bound to E protein monomer (ca. 4910) and E protein dimer (ca. 90 kD). Positive control plasma showed a faint band for prM and capsid (14 kD and 17 kD, not shown) whereas mAbs bound to E protein only.

FIG. 4 indicates that dengue-specific plasmablasts show a distinct VH and VL composition. A) CDR mutation frequency of PBs and MBCs isolated during the acute phase and during convalescence (MBC cony); n.a.: not available. All sequences were IgG and populations were compared using a Mann-Whitney test, **: p=0.005. B) Clonality in MBC and PB populations for patients 10/63 and 10/50 during the acute phase is illustrated in pie charts. The sections represent clones and numbers of sequences per clone are indicated outside the charts. VH sequences were analyzed and were IgG for all sequences except for patient 10/50 MBCs, where IgM and IgG sequences were pooled because most MBCs were IgM. C) Alignment of a VH1-46*01 clone reveals several mutations in each mAb sequence, and some mutations appear to have resulted in loss of dengue specificity (tested by ELISA and histology, FIG. 9). D) V and J family usage of heavy and light chains in patient 10/50. Sequences from all isolated plasmablasts (upper graphs), plasmablasts producing Abs binding to DENV and non-specific memory B cells were grouped into V1 to V6, amongst which the usage of J1 to J6 was determined. All PB sequences were of the IgG isotype, whereas MBCs were of either the IgM or IgG isotype. The numbers of sequences analyzed are indicated in the upper right of the respective graphs.

FIG. 5 illustrates gating strategies for sorting of single plasmablasts or memory B cells. Freshly isolated PBMCs were stained for flow cytometry and the following consecutive gating strategy was used: A) gating on lymphoctyes; B) and C) exclusion of duplets by light scatter characteristics; D) gating on CD20+CD19+ cells and CD20−CD19+ cells. E) CD20+CD19+ cells were further separated according to their CD27 expression and CD27+ memory cells were gated for F) the discrimination between NGC-FITC binding cells (specific memory) and NGC-FITC non-binding cells (unspecific memory). G) CD20−CD19+ cells from D) were gated on CD27high and CD38high cells, and H) were further divided into CD138+ and CD138− cells. Gating as in F) and H) was only applied for patient 10/50. I) to L): as a control for specific binding of NGC-488, a DENV-naive, healthy donor is shown for comparison to D-F. CD38 was not included in the stain and therefore NGC-A488 versus FSC is shown in L). M) Sorted naïve B cells (CD20+CD19+CD27−), memory B cells (CD20+CD19+CD27+) and PB from a dengue patient at day 5 after onset of fever were washed and then directly incubated on anti-IgG or DENV3-coated ELISPOT plates. N) Total IgG and DENV-specific IgG-secreting cells were seen almost exclusively in the sorted PB population.

FIG. 6 shows sequences from all isolated plasmablasts (upper graphs), plasmablasts producing Abs binding to DENV and total memory B cells (CD19+CD27+) from patient 10/63 were grouped into Ig genes using V1 to V6, amongst which the usage of J1 to J6 was determined. All cells were of the IgG isotype. The numbers of sequences analysed are indicated in the upper right of the respective graphs.

FIG. 7 shows binding of antibodies to recombinant E protein or to whole virus particles. A) ELISA plates were coated with whole virus particles that were concentrated by precipitation with Polyethylene glycol and UV-inactivated before storing in aliquots. B) ELISA plates were coated with recombinant E protein (ProspecBio, US). Antibodies indicated in the x-axes were tested, and a starting concentration of 1 ug/ml was used.

FIG. 8 shows a potential format for the detection of DENV-specific antibodies in the serum or plasma of human donors by employing the antibodies described in this patent. DENV-specific antibody in Fab format (to avoid binding of anti-human IgG detection Ab) is used for coating. Virus particles (red) specifically, bind to the coating Abs. Serum samples (orange) from patients or vaccinees are added. Serum antibodies that bind to virus particles are detected with anti-human IgG (blue) conjugated to a tag.

FIG. 9 A) Neutralization assay with DENV-2 at three increasing concentrations of Abs (5, 0.19, and 0.006 μg/ml (27-fold serial dilutions): Each Bi-specific Ab (BisAb) and parent Ab is illustrated with an individual color. Parent antibodies are indicated on the x-axes. Controls: 4G2, a commercially available DENV-neutralizing Ab; AB serum, human serum of blood type AB; no antibody; no virus. B) The same neutralization assay strategy as in (A), but with six increasing concentrations of Abs, starting with 5 μg/ml, then 3-fold serial dilution.

FIG. 10 shows the results of an ELISA indicating that the antibodies do not bind to nonstructural protein NS1. A coating antibody specific for NS1 was used, followed by incubation of the plates with NS1 from infected Vero cells. Antibodies indicated in the x axes were added, and binding antibodies were detected with an anti-human IgG secondary antibody. The positive control was a pool of serum from healthy donors with previous dengue infection.

FIGS. 11 and 12 show neutralizing data for E protein binding antibodies.

FIG. 13 shows neutralizing data for non-E protein binding antibodies.

FIG. 14 illustrates three exemplary competitive ELISA approaches for a situation where the patient has been infected with DENV1 and has generated DENV1-specific antibodies (red). All competition approaches include the coating of a solid surface with EDIII or E protein and binding of patient plasma or serum antibodies, whereas the competitor is A) EDIII, B) a combination of cross-reactive monoclonal human antibodies and C) a set of serotype-specific antibodies.

FIG. 15 shows binding of patient plasma to EDIII. Whereas a EDIII serotype-specific mAb 3H5 binds only to DENV2, plasma from a healthy donor with dengue-immunity binds to EDIII of all serotypes. Shown are serotypes 1 and 2 for illustration.

FIG. 16 provides a comparison of neutralization assay and competition ELISA (approach 1 in FIG. 23) A) Neutralization assay with plasma dilutions ranging from 1:200 to 1:1600. EC50 values at day 2 are 1769, 107, 678, 168 for DENV1, 2, 3 and 4, respectively, suggesting a previous DENV1 or 3 infection. The current infection of this patient is. DENV2, as determined by PCR. B) The plate was coated with EDIII protein of DENV1 and a mixture of plasma plus an increasing amount of competing EDIII protein was added. Only DENV1, and to a lesser extent DENV2 EDIII at increasing concentrations can compete for specific antibodies 17 days after acute secondary DENV2 infection.

FIG. 17 provides results from flow cytometry based competition assay. BHK21 cells infected with DENV are stained with a mixture of 1 ug/ml monoclonal antibodies and increasing dilutions of patient plasma. A) BHK21 cells infected with DENV2 strain TSV01 were stained with a mixture of 1 ug/ml 4G2 and plasma or 9F12 and plasma from a patient ca. 20 days after 1° infection with DENV1. The patient serum can compete with cross-reactive 4G2, but not with 9F12, which is DENV2-specific. B) BHK21 cells infected with DENV3 or DENV4 were stained with a mixture of 1 ug/ml cross-reactive mAb 2C-H3L2 and plasma from a patient with 1° infection DENV1 or a 2° DENV3 infection. Plasma from the patient with 1° infection does not compete with 2C-H3L2, whereas plasma from the patient with 2° infection does compete with 2C-H3L2. Both plasma samples were obtained at early convalescence, about 20 days after fever onset. The patient with 2° DENV3 infection has pre-existing DENV4 antibodies as determined by neutralization assay, explaining the more efficient competition on DENV4-infected cells.

FIG. 18 A) FPLC-SEC chromatography profiles for DENV1-4 rDIII. The sample injected is obtained using step by step dialysis using reducing urea concentration and also in the presence of detergent Tween-20. All four traces correspond to UV absorbance at 280 nm. For clear visualization each profile is represented in different lines and is indicated. Only monomers are obtained for DENV1 to DENV3-rDIII, and aggregates and monomers are observed for DENV4-rDIII. B) Coomassie stained SDS-PAGE gel corresponding to the purified monomers obtained for DENV1-rDIII, DENV2-rDIII, DENV3-rDIII and DENV4-rDIII from FPLC. The expected molecular mass ˜15 KDa is indicated by arrow.

FIG. 19 shows variable loop region identified for EDIII of D1, D2 and D4 based on the available structural data. A) Shows the Multalin results for the sequence of EDIII of D1, D2 and D3 and the non conserved variable region is indicated in the box. B) Comparison of the variable region to the structural data available in the protein data bank. The variable region corresponds to the loop region as shown in the box. This identified variable loop region will be used in generation of serotype specific antibody production which will be then applied in the development of serotype diagnostic kit.

FIG. 20 shows an amino acid sequence alignment of VH and VL regions from plasmablasts 10/50 A) and plasma cells 10/63 B) isolated at day 4 and 6, respectively, after onset of fever. Phylogenetic trees were inferred based on % identity with Jalview software version 11.

FIG. 21 shows an amino acid sequence alignment of VH A) and VL B) regions to from memory B cells of patient 10/50

FIG. 22 shows CDR sequence (CDR1, 2 and 3) in VH and VL regions from plasmablasts of patient 10/63, from plasmablasts from, patient 10/50 and from memory B cells in patient 10/50.

### DEFINITIONS

As used in this application, the singular form “a”, “an” and “the” include plural references unless the context clearly dictates otherwise. For example, the phrase “antibody” also includes a plurality of antibodies.

As used herein, the term “comprising” means “including.” Variations of the word “comprising”, such as “comprise” and “comprises,” have correspondingly varied meanings. Thus, for example, pharmaceutical composition “comprising” an antibody may consist exclusively of that antibody or may include one or more additional components (e.g. another antibody with different binding specificity).

The term “therapeutically effective amount” as used herein, includes within its meaning a non-toxic but sufficient amount of an agent or composition for use in the present invention to provide the desired therapeutic effect. The exact amount required will vary from subject to subject depending on factors such as the species being treated, the age and general condition of the subject, the severity of the condition being treated, the particular agent being administered, the mode of administration and so forth. Thus, it is not possible to specify an exact “effective amount” applicable to all embodiments. However, for any given case, an appropriate “effective amount” may be determined by one of ordinary skill in the art using only routine experimentation.

As used herein, the term “subject” includes any animal of economic, social or research importance including bovine, equine, ovine, primate, avian and rodent species. Hence, a “subject” may be a mammal such as, for example, a human or a non-human mammal.

As used herein, the terms “antibody” and “antibodies” include IgG (including IgG1, IgG2, IgG3, and IgG4), IgA (including IgA1 and IgA2), IgD, IgE, or IgM, and IgY, whole antibodies, including single-chain whole antibodies, and antigen-binding fragments thereof. Antigen-binding antibody fragments include, but are not limited to, Fab, Fab′ and F(ab′)2, Fd, single-chain Fvs (scFv), single-chain antibodies, disulfide-linked Fvs (sdFv) and fragments comprising either a VL or VH domain. The antibodies may be from any animal origin. Antigen-binding antibody fragments, including single-chain antibodies, may comprise the variable region(s) alone or in combination with the entire or partial of the following: hinge region, CH1, CH2, and CH3 domains. Also included are any combinations of variable region(s) and hinge region, CH1, CH2, and CH3 domains. Antibodies may be monoclonal, polyclonal, chimeric, multispecific, humanized, and human monoclonal and polyclonal antibodies which specifically bind the biological molecule.

As used herein, the terms “protein” and “polypeptide” each refer to a polymer made up of amino acids linked together by peptide bonds and are used interchangeably. For the purposes of the present invention a “polypeptide” may constitute a full length protein or a portion of a full length protein.

As used herein, the term “polynucleotide” refers to a single- or double-stranded polymer of deoxyribonucleotide bases, ribonucleotide bases, known analogues or natural nucleotides, or mixtures thereof.

It will be understood that “inducing” an immune response as contemplated herein includes inciting an immune response and enhancing a previously existing immune response.

As used herein the term “treatment”, refers to any and all uses which remedy a disease state or symptoms, or otherwise prevent, hinder, retard, or reverse the progression of disease or other undesirable symptoms in any way whatsoever.

As used herein, the term “kit” refers to any delivery system for delivering materials. Such delivery systems include systems that allow for the storage, transport, or delivery of reaction reagents (for example labels, reference samples, supporting material, etc. in the appropriate containers) and/or supporting materials (for example, buffers, written instructions for performing an assay etc.) from one location to another. For example, kits may include one or more enclosures, such as boxes, containing the relevant reaction reagents and/or supporting materials. The term “kit” includes fragmented kits comprising two or more separate containers that each contains a subportion of the total kit components and combined kits, and combined kits containing all of the components of a reaction assay in a single container (e.g. in a single box housing each of the desired components).

It will be understood that use the term “about” herein in reference to a recited numerical value includes the recited numerical value and numerical values within plus or minus ten percent of the recited value.

It will be understood that use of the term “between” herein when referring to a range of numerical values encompasses the numerical values at each endpoint of the range. For example, a polypeptide of between 10 residues and 20 residues in length is inclusive of a polypeptide of 10 residues in length and a polypeptide of 20 residues in length.

Any description of prior art documents herein, or statements herein derived from or based on those documents, is not an admission that the documents or derived statements are part of the common general knowledge of the relevant art.

For the purposes of description all documents referred to herein are hereby incorporated by reference in their entirety unless otherwise stated.

### DETAILED DESCRIPTION

The following detailed description conveys exemplary embodiments of the present invention in sufficient detail to enable those of ordinary skill in the art to practice the present invention. Features or limitations of the various embodiments described do not necessarily limit other embodiments of the present invention or the present invention as a whole. Hence, the following detailed description does not limit the scope of the present invention, which is defined only by the claims.

Dengue virus has four serotypes, and it is possible for a single individual to be infected four separate times by these alternative strains of virus. Immune protection is specific to the serotype of virus encountered and is thought to persist life-long, whereas secondary infection with a different serotype is associated with an increased risk of severe disease. Successive rounds of infection are associated with increased disease severity, suggesting that pre-existing immunity can have a detrimental effect on the course of disease. The degree and quality of immunity induced against specific dengue subtypes following vaccination and/or initial dengue infection is thus an important consideration in the design of therapeutic and preventative strategies (e.g. re-vaccination).

Knowledge about pre-existing dengue infections is also of relevance for clinicians to decide whether a patient is at higher risk to develop a severe disease. Antibodies with a high affinity for conserved epitopes are generally only present in patients with a secondary infection. Cross-reactive antibodies with suitable binding affinities for multiple dengue virus serotypes can be used as a basis for competitive binding with anti-dengue virus antibodies in patient plasma allowing detection of a previous infection. Alternatively, antibodies with suitable serotype-specific binding properties can be employed to detect whether a patient's antibody repertoire covers all four serotypes. Competition of serum with serotype-specific antibodies will indicate the presence of serum antibodies specific for the respective serotype.

The present invention provides a series of cross-reactive and serotype-specific anti-dengue virus antibodies. The antibodies described herein are advantageous for diagnostic assays such as those described above. Accordingly, methods are provided for diagnosing immunity to dengue, such as immunity arising from previous infection and/or vaccination. In the case where vaccination has occurred, the methods can provide indication as to whether re-vaccination necessary. The present methods may also be used to detect the presence of serotype-specific immunity, thus identifying patients at risk of severe symptoms upon subsequent infection by a different dengue serotype.

The E glycoprotein exposed on the surface of the dengue virus particle is a particularly important target for host neutralizing antibodies. The E protein consists of three domains I, II and III. E domain III (EDIII) sticks out from the virus coat of infectious virus particles and is therefore easily accessible for antibodies. EDIII protein is required for viral attachment to host cells and blocking of EDIII protein by antibodies most efficiently neutralizes the virus. Antibodies blocking other regions of the E protein (EDI and EDII) can also inhibit infection, but the binding of the antibodies is usually of lower affinity and the inhibition less efficient. Surprisingly, the immune response in patients seems to produce many antibodies that are EDII-specific, whereas EDIII-specific antibodies are rare.

Accordingly, the present invention provides a suite of therapeutic antibodies targeting the EDIII domain of the dengue virus E glycoprotein. These antibodies are found to be particularly effective in neutralizing the virus, and can be used for both preventative (e.g. vaccination) and treatment purposes. Also provided herein are a series of bi-specific anti-dengue virus antibodies, which are demonstrated to provide increased neutralization capacity. Methods for the prevention and treatment of dengue virus infection utilizing these antibodies are thus also provided.

### Binding Molecules Against Dengue Virus

The present invention provides molecules capable of binding the dengue virus that are advantageous for the diagnosis, prevention and/or treatment of dengue virus infection.

Nucleotide sequences encoding polypeptides critical to the binding properties of the disclosed molecules are provided in the attached sequence listing (see SEQ ID NOs: 1-108).

Binding molecules of present invention encompass any compound comprising the disclosed dengue-virus specific binding sequences. For example, the binding molecule to may be a polypeptide, fusion protein, minibody, antibody, antibody fragment, or domain deleted antibody.

Binding molecules of the present invention generally bind specifically to particular epitope(s) present on the surface of dengue virus particles. Preferred are binding molecules that bind specifically to the envelope (E) glycoprotein of dengue virus. The binding molecule may bind specifically to the EDI, EDII and/or EDIII domain of the E glycoprotein. In certain embodiments, the binding molecule binds specifically to the EDIII domain.

Binding “specifically” refers to the situation where a given molecule is “specific for” another different molecule. For example, if molecule A binds “specifically” or has binding “specificity” for molecule B, molecule A has the capacity to discriminate between molecule B and any other number of potential alternative binding partners. Accordingly, when exposed to a plurality of different but equally accessible molecules as potential binding partners, molecule A will selectively bind to molecule B and other alternative potential binding partners will remain substantially unbound by molecule A. In general, molecule A will preferentially bind to molecule B at least 10-fold, preferably 50-fold, more preferably 100-fold, and most preferably greater than 100-fold more frequently than other potential binding partners. Molecule A may be capable of binding to molecules that are not molecule B at a weak, yet detectable level. This is commonly known as background binding and is readily discernible from molecule B-specific binding, for example, by use of an appropriate control.

In preferred embodiments, the binding molecules are antibodies or fragments thereof. The antibodies may be polyclonal or monoclonal antibodies. The antibodies may be mammalian antibodies, and preferably human antibodies. In certain embodiments, the antibodies are human monoclonal antibodies.

Non-limiting examples of preferred antibodies with binding specificity for the dengue virus E protein EDIII domain include those referred to in Tables 2A, 2B3A, 3B, 4, 6, and 7. Nucleotide sequences encoding heavy and/or light chain variable domain sequences, including complementarity determining regions (CDR) for these antibodies are provided in the attached Sequence Listing, and designated as SEQ ID NOs: 1-108.

### —Serotype-Specific Antibodies

Antibodies of the present invention may bind specifically to one or more serotype-specific epitope(s) on a given dengue virus particle. Accordingly, the antibodies may display preferential binding specificity for a particular dengue virus serotype or serotypes. For example, the antibodies may display binding specificity for dengue virus serotype 1, dengue virus serotype 2, dengue virus serotype 3, dengue virus serotype 4, or a combination of serotypes 1 and 2; serotypes 1 and 3; serotypes 1 and 4; serotypes 2 and 3; serotypes 2 and 4; serotypes 3 and 4; serotypes 1, 2 and 3; serotypes 1, 2 and 4; or serotypes 2, 3 and 4.

In certain embodiments, the antibodies bind specifically to dengue virus serotype 1. Preferably, the antibodies bind specifically to the dengue virus serotype 1 E protein, and more preferably the EIII domain of the dengue virus serotype 1 E protein. Non-limiting examples of these antibodies include those comprising the CDR sequences encoded by a combination of any one or more of the following heavy and light chain variable domain nucleotide sequences (SEQ ID NO: 7 and SEQ ID NO: 8; SEQ ID NO 43 and SEQ ID NO: 44; SEQ ID NO: 33 and SEQ ID NO: 34). The antibodies may comprise the full heavy/light chain variable domain sequences encoded by SEQ ID NO: 7 and SEQ ID NO: 8; SEQ ID NO 43 and SEQ ID NO: 44; or SEQ ID NO: 34 and SEQ ID NO: 24. The antibodies may comprise CDR sequences from any one or more of the following heavy and light chain variable domain peptide sequences (SEQ ID NO: 153 and SEQ ID NO: 154; SEQ ID NO: 185 and SEQ ID NO: 186; SEQ ID NO: 167 and SEQ ID NO: 168) and/or comprise those full heavy and light chain variable domain peptide sequences.

In other embodiments, the antibodies bind specifically to dengue virus serotype 2. Preferably, the antibodies bind specifically to the dengue virus serotype 2 E protein, and more preferably the EIII domain of the dengue virus serotype 2 E protein. Non-limiting examples of these antibodies include those comprising the CDR sequences encoded by a combination of any one or more of the following heavy and light chain variable domain nucleotide sequences (SEQ ID NO: 29 and SEQ ID NO: 30; SEQ ID NO: 45 and SEQ ID NO: 46; SEQ ID NO: 65 and SEQ ID NO: 66; SEQ ID NO: 79 and SEQ ID NO: 80; SEQ ID NO: 81 and SEQ ID NO: 82; SEQ ID NO: 83 and SEQ ID NO: 84). The antibodies may comprise the full heavy and light chain variable domain sequences encoded by SEQ ID NO: 29 and SEQ ID NO: 30; SEQ ID NO: 45 and SEQ ID NO: 46; SEQ ID NO: 65 and SEQ ID NO: 66; SEQ ID NO: 79 and SEQ ID NO: 80; SEQ ID NO: 81 and SEQ ID NO: 82; or SEQ ID NO: 83 and SEQ ID NO: 84. The antibodies may comprise CDR sequences from any one or more of the following heavy and light chain variable domain peptide sequences (SEQ ID NO: 109 and SEQ ID NO: 110; SEQ ID NO: 111 and SEQ ID: NO 112; SEQ ID NO: 119 and SEQ ID NO: 120: SEQ ID NO: 149 and SEQ ID NO: 150; SEQ ID NO: 193 and SEQ ID NO: 194; SEQ ID NO: 195 and SEQ ID NO: 196) to and/or comprise those full heavy and light chain variable domain peptide sequences. In still other embodiments, the antibodies bind specifically to dengue virus serotype 3. Preferably, the antibodies bind specifically to the dengue virus serotype 3 E protein, and more preferably the EIII domain of the dengue virus serotype 3 E protein. Non-limiting examples of these antibodies include those comprising the CDR sequences encoded by a combination of any one or more of the following heavy and light chain variable domain nucleotide sequences (SEQ ID NO: 75 and SEQ ID NO: 76). The antibodies may comprise the full heavy and light chain variable domain sequences encoded by SEQ ID NO: 75 and SEQ ID NO: 76. The antibodies may comprise CDR sequences from any one or more of the following heavy and light chain variable domain peptide sequences (SEQ ID NO: 125 and SEQ ID NO: 126) and/or comprise those full heavy and light chain variable domain peptide sequences.

In other additional embodiments, the antibodies bind specifically to both dengue virus serotype 1 and dengue virus serotype 2. Preferably, the antibodies bind specifically to the dengue virus serotype 1 and 2 E proteins, and more preferably the EIII domain of the dengue virus serotype 1 and 2 E proteins. Non-limiting examples of these antibodies include those encoded by a combination of the following heavy and light chain variable domain nucleotide sequences (SEQ ID NO: 85 and SEQ ID NO: 86). The antibodies may comprise the full heavy and light chain variable domain sequences encoded by SEQ ID NO: 85 and SEQ ID NO: 86. The antibodies may comprise CDR sequences from any one or more of the following heavy and light chain variable domain peptide sequences (SEQ ID NO: 125 and SEQ ID NO: 126 and/or comprise those full heavy and light chain variable domain peptide sequences.

In further additional embodiments, the antibodies bind specifically to both dengue virus serotype 1 and dengue virus serotype 3. Preferably, the antibodies bind specifically to the dengue virus serotype 1 and 3 E proteins, and more preferably the EIII domain of the dengue virus serotype 1 and 3 E proteins. Non-limiting examples of these antibodies include those encoded by a combination of the following heavy and light chain variable domain nucleotide sequences (SEQ ID NO: 33 and SEQ ID NO: 34; SEQ ID NO: 87 and SEQ ID NO: 88). The antibodies may comprise the full heavy and light chain variable domain sequences encoded by SEQ ID NO: 33 and SEQ ID NO: 34; or SEQ ID NO: 87 and SEQ ID NO: 88. The antibodies may comprise CDR sequences from any one or more of the following heavy and light chain variable domain peptide sequences (SEQ ID NO: 175 and SEQ ID NO: 176; SEQ ID NO: 205 and SEQ ID NO: 206) and/or comprise those full heavy and light chain variable domain peptide sequences.

Serotype-specific antibodies of the present invention may bind specifically to dengue virus serotype(s) with particularly high affinity, and in certain embodiments with higher affinity than cross-reactive antibodies. The serotype-specific antibodies may be particularly suited to use as neutralizing antibodies. Non-limiting examples, of serotype-specific antibodies of the present invention that display strong affinity for dengue virus serotype(s) and which are thus suitable neutralizing antibodies include those antibodies designated herein as 5D-H1L2, 2C-H3L2, 6C-H8L1, 8F-H1L1, 5A-H6L1, 11E-H1L1, 6E-H1L1, 1B-H1L1, 9B-H1L1.

Further non-limiting examples include those antibodies designated herein as A3-3M2-D3-H2L1, A3-3M2-F5-H1L2, A3-3M3-C4-H3L3 and A3-2M2-G8-H1L2.

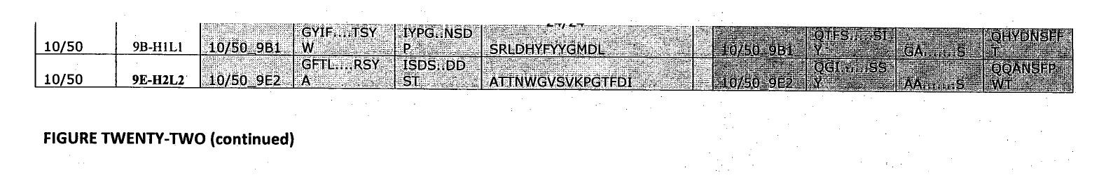

Serotype-specific antibodies in accordance with the present invention may have a series of heavy and light chain variable domain CDR sequences in accordance with any one clone as designated in FIG. 22 herein.

### —Cross-Reactive Antibodies

Antibodies of the present invention may be cross-reactive in that they may have a capacity to bind specifically to two, three or four serotypes of dengue virus. In preferred embodiments, cross-reactive antibodies of the present invention bind specifically to four different dengue virus serotypes. The binding to each of the four serotypes may be of identical or substantially identical affinity.

By way of non-limiting example only, cross-reactive antibodies of the present invention may have binding affinity to a target epitope of a dengue virus particle comparable to those of antibodies generated by memory B lymphocytes upon re-infection with dengue virus, or infection after vaccination. Cross-reactive antibodies in this genre may be referred to herein as “high affinity” cross-reactive antibodies. Non-limiting examples of these antibodies include those encoded by a combination of any one or more of the following heavy and light chain variable domain nucleotide sequences (SEQ ID NO: 89 and SEQ ID NO: 90; SEQ ID NO: 91 and SEQ ID NO: 92; SEQ ID NO: 93 and SEQ ID NO: 94; SEQ ID NO: 95 and SEQ ID NO: 96; SEQ ID NO: 97 and SEQ ID NO: 98; SEQ ID NO: 99 and SEQ ID NO: 100; SEQ ID NO: 101 and SEQ ID NO: 102; SEQ ID NO: 103 and SEQ ID NO: 104; SEQ ID NO: 105 and SEQ ID NO: 106; SEQ ID NO: 107 and SEQ ID NO: 108). The antibodies may comprise the full heavy and light chain variable domain sequences encoded by SEQ ID NO: 89 and SEQ ID NO: 90; SEQ ID NO: 91 and SEQ ID NO: 92; SEQ ID NO: 93 and SEQ ID NO: 94; SEQ ID NO: 95 and SEQ ID NO: 96; SEQ ID NO: 97 and SEQ ID NO: 98; SEQ ID NO: 99 and SEQ ID NO: 100; SEQ ID NO: 101 and SEQ ID NO: 102; SEQ ID NO: 103 and SEQ ID NO: 104; SEQ ID NO: 105 and SEQ ID NO: 106; or SEQ ID NO: 107 and SEQ ID NO: 108. The antibodies may comprise CDR sequences from any one or more of the following heavy, and light chain variable domain peptide sequences (SEQ ID NOs: 189 and 190; SEQ ID NOs: 191 and 192; SEQ ID NOs: 199 and 200; SEQ ID NOs: 201 and 202: SEQ ID NOs: 203 and 204; SEQ ID NOs: 207 and 208; SEQ ID NOs: 209 and 210; SEQ ID NOs: 211 and 212; SEQ ID NOs: 213 and 214; SEQ ID NOs: 215 and 216) and/or comprise those full heavy and light chain variable domain peptide sequences.

“High affinity” cross-reactive antibodies of the present invention may bind specifically to multiple dengue virus serotypes with particularly high affinity and thus may be particularly suited to use as neutralizing antibodies. Non-limiting examples, of “high affinity” cross-reactive antibodies of the present invention that display strong affinity for dengue virus serotype(s) and which are thus suitable neutralizing antibodies include those antibodies designated herein as A3-3M1-B11-H1L2, A3-3M1-D1-H1L3, A3-3M3-G3-H1L1, A3-2M1-A5-H1L1, A3-2M2-H5-H3L2, A3-2M3-C9-H2L1, A3-2M3-E9-H1L2, A3-2M3-F9-H2L1, A3-2M3-G3-H2L3, A3-2M3-C2-H3L1, and 5D-H6L2. Another non-limiting example is the antibody designated herein as 5D-H6L2.

By way of further non-limiting example, cross-reactive antibodies of the present invention may have binding affinity to a target epitope of a dengue virus particle comparable to those of antibodies generated by plasma cells or plasmablasts upon primary infection or initial vaccination with dengue virus. Cross-reactive antibodies in this genre may be referred to herein as “low affinity” cross-reactive antibodies. Non-limiting examples of these antibodies include those encoded by a combination of any one or more of the following heavy and light chain variable domain nucleotide sequences (SEQ ID NO: 1 and SEQ ID NO: 2; SEQ ID NO: 3 and SEQ ID NO: 4; SEQ ID NO: 5 and SEQ ID NO: 6; SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; SEQ ID NO: 25 and SEQ ID NO: 26; SEQ ID NO: 27 and SEQ ID NO: 28; SEQ ID NO: 31 and SEQ ID NO: 32; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 47 and SEQ ID NO: 48; SEQ ID NO: 49 and SEQ ID NO: 50; SEQ ID NO: 51 and SEQ ID NO: 52; SEQ ID NO: 53 and SEQ ID NO: 54; SEQ ID NO: 55 and SEQ ID NO: 56; SEQ ID NO: 57 and SEQ ID NO: 58; SEQ ID NO: 59 and SEQ ID NO: 60; SEQ ID NO: 61 and SEQ ID NO: 62; SEQ ID NO: 63 and SEQ ID NO: 64; SEQ ID NO: 65 and SEQ ID NO: 66; SEQ ID NO: 67 and SEQ ID NO: 68; SEQ ID NO: 69 and SEQ ID NO: 70; SEQ ID NO: 71 and SEQ ID NO: 72; SEQ ID NO: 73 and SEQ ID NO: 74; SEQ ID NO: 77 and SEQ ID NO: 78). The antibodies may comprise the full heavy and light chain variable domain sequences encoded by SEQ ID NO: 1 and SEQ ID NO: 2; SEQ ID NO: 3 and SEQ ID NO: 4; SEQ ID NO: 5 and SEQ ID NO: 6; SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; SEQ ID NO: 25 and SEQ ID NO: 26; SEQ ID NO: 27 and SEQ ID NO: 28; SEQ ID NO: 31 and SEQ ID NO: 32; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 47 and SEQ ID NO: 48; SEQ ID NO: 49 and SEQ ID NO: 50; SEQ ID NO: 51 and SEQ ID NO: 52; SEQ ID NO: 53 and SEQ ID NO: 54; SEQ ID NO: 55 and SEQ ID NO: 56; SEQ ID NO: 57 and SEQ ID NO: 58; SEQ ID NO: 59 and SEQ ID NO: 60; SEQ ID NO: 61 and SEQ ID NO: 62; SEQ ID NO: 63 and SEQ ID NO: 64; SEQ ID NO: 65 and SEQ ID NO: 66; SEQ ID NO: 67 and SEQ ID NO: 68; SEQ ID NO: 69 and SEQ ID NO: 70; SEQ ID NO: 71 and SEQ ID NO: 72; SEQ ID NO:573 and SEQ ID NO: 74; or SEQ ID NO: 77 and SEQ ID NO: 78. The antibodies may comprise CDR sequences from any one or more of the following heavy and light chain variable domain peptide sequences (SEQ ID NOs: 113 and 114; SEQ ID NOs: 115 and 116; SEQ ID NOs: 121 and 122; SEQ ID NOs: 123 and 124; SEQ ID NOs: 127 and 128; SEQ ID NOs: 129 and 130; SEQ ID NOs: 131 and 132; SEQ ID NOs: 133 and 134; SEQ ID NOs: 135 and 136; SEQ ID NOs: 137 and 138; SEQ ID NOs: 139 and 140; SEQ ID NOs: 141 and 142; SEQ ID NOs: 143 and 144; SEQ ID NOs: 145 and 146; SEQ ID NOs: 147 and 148; SEQ ID NOs: 151 and 152; SEQ ID NOs: 153 and 154; SEQ ID NOs: 155 and 156; SEQ ID NOs: 157 and 158; SEQ ID NOs: 159 and 160; SEQ ID NOs: 161 and 162; SEQ ID NOs: 163 and 164; SEQ ID NOs: 165 and 166; SEQ ID NOs: 169 and 170; SEQ ID NOs: 171 and 172; SEQ ID NOs: 173 and 174; SEQ ID NOs: 177 and 178; SEQ ID NOs: 179 and 180; SEQ ID NOs: 181 and 182; SEQ ID NOs: 183 and 184; SEQ ID NOs: 187 and 188; SEQ ID NOs: 189 and 190; SEQ ID NOs: 191 and 192; SEQ ID NOs: 193 and 104: SEQ ID NOs: 195 and 196; SEQ ID NOs: 197 and 198; SEQ ID NOs: 199 and 200; SEQ ID NOs: 201 and 202; SEQ ID NOs: 203 and 204; SEQ ID NOs: 205 and 206; SEQ ID NOs: 207 and 208; SEQ ID NOs: 209 and 210; SEQ ID NOs: 211 and 212; SEQ ID NOs: 213 and 214; SEQ ID NOs: 215 and 216) and/or comprise those full heavy and light chain variable domain peptide sequences.

Cross-reactive antibodies in accordance with the present invention may have a series of heavy and light chain variable domain CDR sequences in accordance with any one clone as designated in FIG. 22 herein.

### —Bispecific Binding Molecules,

A bispecific binding molecule of the present invention may comprise one binding site that reacts with a first target epitope of a dengue virus and one binding site that reacts with a second target epitope of the same or a separate dengue virus. Non-limiting examples of bispecific molecules include bispecific antibodies bispecific fusion proteins and bispecific minibodies.

In preferred embodiments, the bispecific binding molecule is a bispecific antibody. The bispecific antibodies may be generated using known methods such as, for example, by linking the variable domain of a given antibody with the variable domain of a different antibody using a suitable linker such as a protein linker. Bispecific antibodies may be expressed in a suitable cell line (e.g. HEK cells) and tested for their capacity to neutralize dengue infection in an appropriate assay (e.g. fluorescene-based neutralization assay as described in the Examples herein).

Non-limiting examples of suitable bispecific antibodies include those comprising heavy chain and/or light chain variable domain CDR sequences derived from the specific antibody combinations shown in Tables 5 and Table 6. Nucleotide sequences encoding the heavy chain and/or light chain variable domain CDR sequences derived from the specific antibody combinations shown in Tables 5 and Table 6 are shown in Sequence Listing.

### Preventative and Therapeutic Methods

The present invention provides methods for preventing and/or treating a dengue virus infection in a subject. Also provide are methods for inducing an immune response against the dengue virus in a subject. The methods may be conducted for prophylactic, ameliorative, palliative, and/or therapeutic purposes to induce an immune response against, or, prevent or treat infection by, a Dengue virus. The Dengue virus may be a serotype I, II, III, or IV virus, or a recombinant form thereof.

The methods of the present invention may be used to induce an immune response against dengue virus in a subject. It will be understood that “inducing” an immune response as contemplated herein includes inciting an immune response and modulating a previously existing immune response (e.g. enhancing or existing immune response). “Enhancing” an immune response as contemplated herein refers to augmenting the immune response such as, for example, innate immunity and/or adaptive immunity (e.g. humoral responses) in a subject, for example, against a given serotype or serotype(s) of dengue virus. Methods for measuring the immune response are known to persons of ordinary skill in the art. Exemplary methods include solid-phase heterogeneous assays (e.g. enzyme-linked immunosorbent, assay), solution phase assays (e.g. electrochemiluminescence assay), amplified luminescent proximity homogeneous assays, flow cytometry, intracellular cytokine staining, functional T-cell assays, functional B-cell assays, functional monocyte-macrophage assays, dendritic and reticular endothelial cell assays, measurement of NK cell responses, oxidative burst assays, cytotoxic-specific cell lysis assays, pentamer binding assays, and phagocytosis and apoptosis evaluation.

“Subjects” as contemplated herein include mammals (e.g. humans) and individuals of any species of social, economic or research importance including, but not limited to, ovine, bovine, equine, porcine, feline, canine, avian, primate, and rodent species.

The methods comprise administering to the subject a binding molecule of the present invention. Preferably, the binding molecule is an antibody such as, for example, a human monoclonal antibody. The antibody may be a component of a pharmaceutical composition (e.g. a vaccine). Non-limiting examples of suitable antibodies are described above in the section entitled “Binding molecules against dengue virus”.

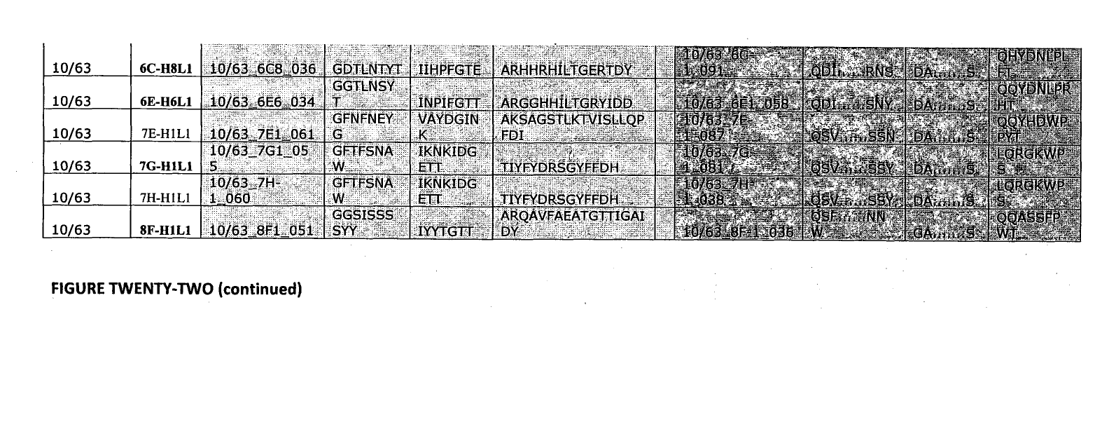

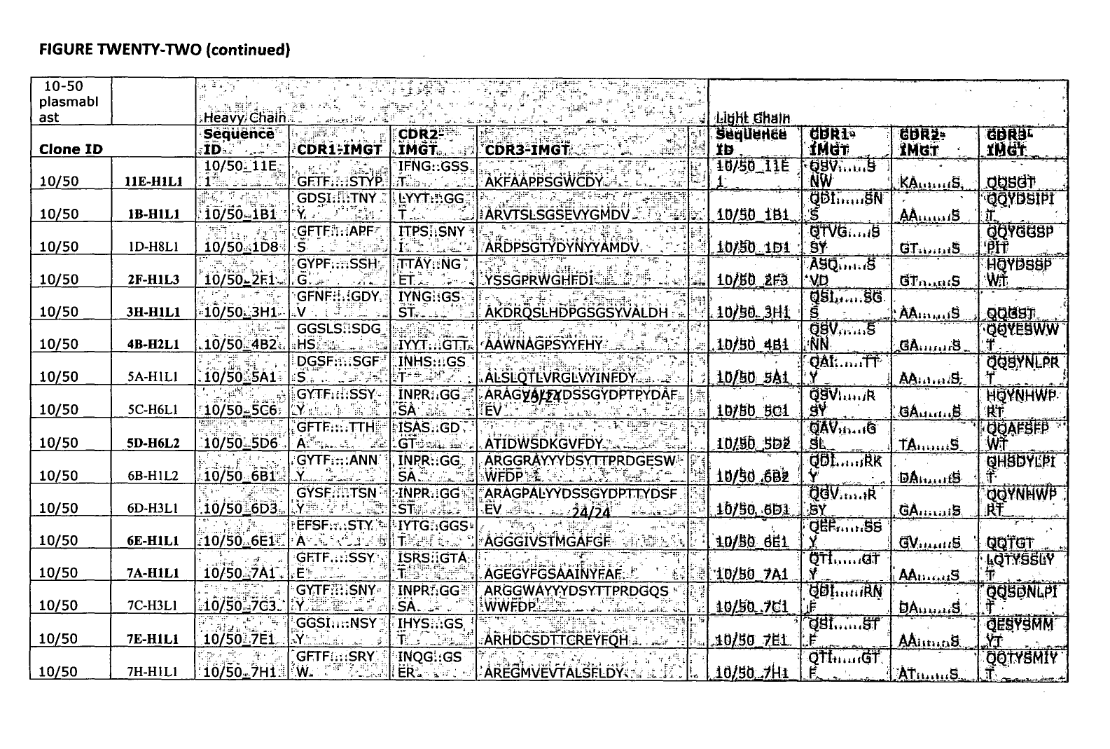

An antibody administered in accordance with the methods may comprise the heavy and/or light chain variable domain CDR sequence(s) of a single antibody as defined in FIG. 20. The heavy and/or light chain variable domain CDR sequence(s) may be encoded by a nucleotide sequence as defined in any one of SEQ ID NOs: 1-108. The antibody administered may comprise one or more of the full heavy/light chain variable domain sequences encoded by any one or SEQ ID NOs: 1-108.

Non-limiting examples of cross-reactive and serotype-specific antibodies of the present invention that may be particularly suited to therapeutic applications include those antibodies designated herein as A3-3M1-B11-H1L2, A3-3M1-D1-H1L3, A3-3M3-G3-H1L1, A3-2M1-A5-H1L1, A3-2M2-H5-H3L2, A3-2M3-C9-H2L1, A3-2M3-E9-H1L2, A3-2M3-F9-H2L1, A3-2M3-G3-H2L3, A3-2M3-C2-H3L1, A3-3M2-D3-H2L1, A3-3M2-F5-H1L2, A3-3M3-C4-H3L3, A3-2M2-G8-H1L2, 5D-H1L2, 2C-H3L2, 6C-H8L1, 8F-H1L1, 5A-H6L1, 11E-H1L1, 6E-H1L1, 1B-H1L1, and 9B-H1L15D-H6L2. The antibodies may be those encoded by a combination of any one or more of the following heavy and light chain variable domain nucleotide sequences, or may comprise the full heavy and light chain variable domain sequences encoded by any one of the following sequences: SEQ ID NO: 7 and SEQ ID NO: 8; SEQ ID NO: 33 and SEQ ID NO: 34; SEQ ID NO 43 and SEQ ID NO: 44; SEQ ID NO: 34 and SEQ ID NO: 24; SEQ ID NO: 29 and SEQ ID NO: 30; SEQ ID NO: 45 and SEQ ID NO: 46; SEQ ID NO: 59 and SEQ ID NO:60; SEQ ID NO: 65 and SEQ ID NO: 66; SEQ ID NO: 75 and SEQ ID NO: 76; SEQ ID NO: 79 and SEQ ID NO: 80; SEQ ID NO: 81 and SEQ ID NO: 82; SEQ ID NO: 83 and SEQ ID NO: 84; SEQ ID NO: 85 and SEQ ID NO: 86; SEQ ID NO: 87 and SEQ ID NO: 88; SEQ ID NO: 89 and SEQ ID NO: 90; SEQ ID NO: 91 and SEQ ID NO: 92; SEQ ID NO: 93 and SEQ ID NO: 94; SEQ ID NO: 95 and SEQ ID NO: 96; SEQ ID NO: 97 and SEQ ID NO: 98; SEQ ID NO: 99 and SEQ ID NO: 100; SEQ ID NO: 101 and SEQ ID NO: 102; SEQ ID NO: 103 and SEQ ID NO: 104; SEQ ID NO: 105 and SEQ ID NO: 106; or SEQ ID NO: 107 and SEQ ID NO: 108. The antibodies may comprise CDR sequences from any one or more of the following heavy and light chain variable domain peptide sequences (SEQ ID NOs: 109 and 110; SEQ ID NOs: 111 and 112; SEQ ID NOs: 117 and 118; SEQ ID NOs: 119 and 120; SEQ ID NOs: 125 and 126; SEQ ID NOs: 149 and 150; SEQ ID NOs: 153 and 154; SEQ ID NOs: 167 and 168; SEQ ID NOs: 175 and 176; SEQ ID NOs: 185 and 186; SEQ ID NOs: 189 and 190; SEQ ID NOs: 191 and 192; SEQ ID NOs: 193 and 194; SEQ ID NOs: 195 and 196; SEQ ID NOs: 197 and 198; SEQ ID NOs: 199 and 200; SEQ ID NOs: 201 and 202: SEQ ID NOs: 203 and 204; SEQ ID NOs: 205 and 206; SEQ ID NOs: 207 and 208; SEQ ID NOs: 209 and 210; SEQ ID NOs: 211 and 212; SEQ ID NOs: 213 and 214; SEQ ID NOs: 215 and 216) and/or comprise those full heavy and light chain variable domain peptide sequences. Therapeutic antibodies in accordance with the present invention may have a series of heavy and light chain variable domain CDR sequences in accordance with any one clone as designated in FIG. 22 herein.

These antibodies may also be administered as immunogenic vaccines or components of such vaccines.

Additional non-limiting examples of binding molecules that may be particularly suited to therapeutic applications include bispecific binding molecules antibodies such as those described in the subsection above entitled “bispecific binding molecules”. Preferably, the bispecific binding molecules are bispecific antibodies.

The binding molecules (e.g. antibodies) can be administered in the form of a pharmaceutical composition.

In certain embodiments, the pharmaceutical compositions are vaccines. Vaccines of the present invention include both preventative vaccines (i.e. vaccines administered for the purpose of preventing infection) and therapeutic vaccines (i.e. vaccines administered for the purpose of treating infection). A vaccine of the present invention may therefore be administered to a recipient for prophylactic, ameliorative, palliative, or therapeutic purposes.

Pharmaceutical compositions of the present invention may be prepared using methods known to those of ordinary skill in the art. Non-limiting examples of suitable methods are described in Gennaro et al. (Eds), (1990), “Remington's Pharmaceutical Sciences”, Mack Publishing Co., Easton, Pa., USA.

Pharmaceutical compositions of the present invention may comprise a pharmaceutically acceptable carrier, excipient, diluent and/or adjuvant. “Pharmaceutically acceptable” carriers, excipients, diluents and/or adjuvants as contemplated herein are substances which in general do not produce significant adverse reaction(s) when administered to a particular recipient such as a human or non-human animal. Pharmaceutically acceptable carriers, excipients, diluents, and adjuvants are generally also compatible with other ingredients of the composition. Non-limiting examples of suitable excipients, diluents, and carriers can be found in the “Handbook of Pharmaceutical Excipients” 4th Edition, (2003) Rowe et al. (Eds), The Pharmaceutical Press, London, American Pharmaceutical Association, Washington.

Pharmaceutical compositions of the present invention may be in a form suitable for administration by injection, in a formulation suitable for oral ingestion (such as, for example, capsules, tablets, caplets, elixirs), in the form of an ointment, cream or lotion suitable for topical administration, in a form suitable for delivery as an eye drop, in an aerosol form suitable for administration by inhalation, such as by intranasal inhalation or oral inhalation, or in a form suitable for parenteral administration, that is, subcutaneous, intramuscular or intravenous injection.

Supplementary active ingredients such as adjuvants or biological response modifiers can also be incorporated into pharmaceutical compositions of the present invention. Although adjuvant(s) may be included in pharmaceutical compositions of the present invention they need not necessarily comprise an adjuvant. In such cases, reactogenicity problems arising from the use of adjuvants may be avoided.

In general, adjuvant activity in the context of a pharmaceutical composition of the present invention includes, but is not limited to, an ability to enhance the immune response (quantitatively or qualitatively) induced by immunogenic components in the composition (e.g. an antibody of the present invention). This may reduce the dose or level of the immunogenic components required to produce an immune response and/or reduce the number or the frequency of immunisations required to produce the desired immune response.

Any suitable adjuvant may be included in a pharmaceutical composition of the present invention. For example, an aluminium-based adjuvant may be utilised. Suitable aluminium-based adjuvants include, but are not limited to, aluminium hydroxide, aluminium phosphate and combinations thereof. Other specific examples of aluminium-based adjuvants that may be utilised are described in European Patent No. 1216053 and U.S. Pat. No. 6,372,223. Other suitable adjuvants include Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminium salts such as aluminium hydroxide gel (alum) or aluminium phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A; oil in water emulsions including those described in European Patent No. 0399843, U.S. Pat. No. 7,029,678 and PCT Publication No. WO 2007/006939; and/or additional cytokines, such as GM-CSF or interleukin-2, -7, or -12, granulocyte-macrophage colony-stimulating factor (GM-CSF), monophosphoryl lipid A (MPL), cholera toxin (CT) or its constituent subunit, heat labile enterotoxin (LT) or its constituent subunit, toll-like receptor ligand adjuvants such as lipopolysaccharide (LPS) and derivatives thereof (e.g. monophosphoryl lipid A and 3-Deacylated monophosphoryl lipid A), muramyl dipeptide (MDP) and F protein of Respiratory Syncytial Virus (RSV).

Pharmaceutical compositions of the present invention (e.g. vaccines) may be provided in a kit. The kit may comprise additional components to assist in performing the methods of the present invention such as, for example, administration device(s), buffer(s), and/or diluent(s). The kits may include containers for housing the various components and instructions for using the kit components in the methods of the present invention.

In general, an antibody or pharmaceutical composition of the present invention is administered to the subject in a therapeutically effective amount. A “therapeutically effective amount” includes within its meaning a non-toxic but sufficient amount of an antibody or pharmaceutical composition for use in the present, invention to provide the desired therapeutic effect. The exact amount required will vary from subject to subject depending on factors such as the species being treated, the age and general condition of the subject, the severity of the condition being treated, the nature of the infection being prevented, the particular agent being administered, the mode of administration, and so forth. Thus, it is not possible to specify an exact “therapeutically effective amount”. However, for any given case, an appropriate “therapeutically effective amount” may be determined by one of ordinary skill in the art using only routine experimentation.

A pharmaceutical composition of the present invention can be administered to a recipient by standard routes, including, but not limited to, parenteral (e.g. intravenous, intraspinal, subcutaneous or intramuscular), oral, topical, or mucosal routes (e.g. intranasal).

A pharmaceutical composition of the present invention may be administered to a recipient in isolation or in combination with other additional therapeutic agent(s). In embodiments where a pharmaceutical composition is administered with therapeutic agent(s), the administration may be simultaneous or sequential (i.e. pharmaceutical composition administration followed by administration of the agent(s) or vice versa).

In general, a pharmaceutical composition of the present invention can be administered in a manner compatible with the route of administration and physical characteristics of the recipient (including health status) and in such a way that it elicits the desired effect(s) (i.e. therapeutically effective, immunogenic and/or protective). For example, the appropriate dosage of a pharmaceutical composition of the present invention may depend on a variety of factors including, but not limited to, a subject's physical characteristics (e.g. age, weight, sex), whether the compound is being used as single agent or adjuvant therapy, the type of MHC restriction of the patient, the progression (i.e. pathological state) of a virus infection, and other factors that may be recognized by one skilled in the art. Various general considerations that may be considered when determining an appropriate dosage of a pharmaceutical composition of the present invention are described, for example, in Gennaro et al. (Eds), (1990), “Remington's Pharmaceutical Sciences”, Mack Publishing Co., Easton, Pa., USA; and Gilman et al., (Eds), (1990), “Goodman And Gilman's: The Pharmacological Bases of Therapeutics”, Pergamon Press.

One skilled in the art would be able, by routine experimentation, to determine an effective, non-toxic amount of an antibody described herein to include in a pharmaceutical composition of the present invention for the desired therapeutic outcome. In general, a pharmaceutical composition of the present invention may be administered to a patient in an amount of from about 50 micrograms to about 5 mg of active component(s). Dosage in an amount of from about 50 micrograms to about 500 micrograms is especially preferred. Generally, an effective dosage is expected to be in the range of about 0.0001 mg to about 1000 mg of active component(s) per kg body weight per 24 hours; typically, about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours; or about 1.0 mg to about 250 mg per kg body weight per 24 hours. More typically, an effective dose range is expected to be in the range about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; or about 5.0 mg to about 15 mg per kg body weight per 24 hours.

Typically, in treatment applications, the treatment may be for the duration of the disease state or condition. Further, it will be apparent to one of ordinary skill in the art that the optimal quantity and spacing of individual dosages will be determined by the nature and extent of the disease state or condition being treated, the form, route and site of administration, and the nature of the particular individual being treated. Optimum conditions can be determined using conventional techniques.

In many instances (e.g. preventative applications), it may be desirable to have several or multiple administrations of a pharmaceutical composition of the present invention. For example, a pharmaceutical composition may be administered 1, 2, 3, 4, 5, 6, 7, 8, 9, 10, or more times. The administrations may be from about one to about twelve week intervals, and in certain embodiments from about one to about four week intervals. Periodic re-administration may be desirable in the case of recurrent exposure to dengue virus targeted by a pharmaceutical composition of the present invention.

It will also be apparent to one of ordinary skill in the art that the optimal course of administration can be ascertained using conventional course of treatment determination tests.

Where two or more entities are administered to a subject “in conjunction”, they may be administered in a single composition at the same time, or in separate compositions at the same time, or in separate compositions separated in time.

Certain embodiments of the present invention involve the administration of pharmaceutical compositions in multiple separate doses. Accordingly, the methods for the prevention (i.e. vaccination) and treatment of dengue virus infection described herein encompass the administration of multiple separated doses to a subject, for example, over a defined period of time. Accordingly, the methods for the prevention (i.e. vaccination) and treatment of infection disclosed herein include administering a priming dose of a pharmaceutical composition of the present invention. The priming dose may be followed by a booster dose. The booster may be for the purpose of re-vaccination. In various embodiments, the pharmaceutical composition or vaccine is administered at least once, twice, three times or more.

### Diagnostic Methods

Antibodies of the present invention may be used for diagnostic methods. The diagnostic methods may be used for a variety of different purposes such as, for example, diagnosing immunity to dengue, diagnosing immunity to dengue in patients having a pre-existing immunity to dengue, determining efficacy of vaccination against dengue, determining the efficacy of vaccination against dengue across different serotypes in a defined population or in an individual, determining the presence of at least one type of antibody directed against a dengue virus in an individual or a population having been already previously infected with dengue, determining dengue virus serotype-specific immunity after vaccination against dengue and/or determining when re-vaccination against dengue is necessary.

In certain embodiments, the diagnostic methods utilise competitive antibody binding for a target molecule (e.g. a dengue virus E protein). For example, the presence or absence of certain cross-specific and/or serotype-specific anti-dengue antibodies in a biological sample from a subject (e.g. blood plasma or any other suitable body fluid) may be detected by coating dengue virus or targeted components thereof (e.g. E protein, or domain(s) thereof) with an antibody of the present invention. By way of non-limiting example only, a “low affinity” cross-reactive antibody of the present invention (see non-limiting examples in section above entitled “Cross-reactive antibodies” may be applied to a dengue viral E protein which is bound directly or indirectly (e.g. via another antibody) to the surface of an insoluble support. The support might be, for example, a bead or the well of a culture plate. Multiple supports may be used, and different supports might be coated with dengue virus E protein derived from different serotypes. A biological sample from the patient is applied, and the presence of high affinity and/or serotype-specific antibodies against the dengue virus can be detected if competition is detected between the low affinity antibodies originally applied to the E protein and any antibodies with similar binding specificities in the sample. Competitive removal of the low affinity antibodies from the E protein by higher-affinity antibodies present in the sample may alter detectable signal, for example, when either type of antibody comprises a detectable label and results are compared with controls. The detectable, label may be measured by known techniques such as ELISA and/or flow cytometry.

In other embodiments, the diagnostic methods utilise capture assays in which, for example, whole dengue virus which is bound directly or indirectly (e.g. via another antibody) to the surface of an insoluble support (e.g. bead or well of a plate). Multiple supports may be used, and different supports might be coated with dengue virus E protein derived from different serotypes. Antibodies specific for the bound virus in a biological sample can be detected upon their “capture” by the bound virus particles culminating in a detectable signal. The captured antibodies may be detected using any suitable technique (e.g. application of a labelled secondary antibody) and signal measured by a suitable method (e.g. ELISA and/or flow cytometry).

In still other embodiments, the diagnostic methods utilize competitive binding for a given serotype of dengue virus E protein (e.g. EDIII domain) between detectably-labeled serotype-specific antibodies of the present invention (see suitable examples in the subsection above entitled “serotype-specific antibodies”) and serotype-specific antibodies in a biological sample from a patient. The detectable label may be measured by known techniques such as ELISA and/or flow cytometry.

Biosensors may also be used in the diagnostic methods of the present invention.

Non-limiting examples of specific diagnostic assays utilizing antibodies of the present invention are described in the Examples and Figures of the specification.

In certain embodiments the present invention provides an assay for determining efficacy of vaccination against dengue. The assay can be used to determine efficacy of vaccination against dengue across different serotypes in a defined population or in an individual. This assay can be carried out either after the vaccination, or after an infection with dengue. In particular, the assay allows identifying dengue serotype-specific immunity in a population or an individual.

Also provided is an assay for determining the presence of at least one type of antibody directed against a dengue virus in an individual or a population having been already previously infected with dengue, wherein the at least one type of antibody is determined within 1 hour, or 10 hours, or 20 hours, or 30 hours, or 40 hours, or 50 hours, or 60 hours, or 70 hours, or 72 hours after onset of dengue fever being the result of a second or further infection with dengue in the individual or the individuals of a population, or between onset of dengue fever to 72 hours after onset of dengue fever or before production of new antibodies against dengue of the second or further infection with dengue in the individual or the individuals of a population.

It will be appreciated by persons of ordinary skill in the art that numerous variations and/or modifications can be made to the present invention as disclosed in the specific embodiments without departing from the spirit or scope of the present invention as broadly described. The present embodiments are, therefore, to be considered in all respects as illustrative and not restrictive.

### EXAMPLES

The present invention will now be described with reference to specific examples, which should not be construed as in any way limiting.

### Example 1

### Plasmablasts Generated During Repeated Dengue Infection are Virus Glycoprotein-Specific and Bind to Multiple Virus Serotypes

### 1.1 Material and Methods

### —Patients

The research involving patients enrolled in the EDEN study was approved by the Institutional Review Board of Singapore National Healthcare Group Ethical Domain (DSRB B/05/013) and patients gave written informed consent. As part of the collaborative program “STOP Dengue” (www.stopdengue.sg), adult patients (age >21 years) presenting at community primary care clinics with acute onset fever (>38.5° C. for less than 72 h) without rhinitis or clinically obvious alternative diagnoses were included in the study. Whole blood samples were collected into EDTA-vacutainer tubes (Becton Dickinson) at recruitment (acute phase), at 4-7 days (defervescence), and 3-4 weeks after fever onset (convalescence). Patients were diagnosed by DENV-specific RT-PCR. DENV-specific IgM and IgG antibodies were detected by ELISA using the commercially available PanBio kit (Inverness Medical, Australia). Both patients described in this study had DENV-specific serum IgG antibodies at the time of fever onset and were therefore classified as having secondary infections.

### —Cell Lines and Virus Strains

All viruses used were produced in C6/36 mosquito cells (ATCC). Patient isolate strains were used for plasmablast screening ELISA and neutralization assays: DENV1-05K2916 (EU081234 (Schreiber et al., 2009), DENV2-TSV01 (McBride and Vasudevan, 1995), DENV3-VN32/96 (EU482459) and DENV4-My04 31580. DENV3 and 4 were provided by Dr. Cameron Simmons, Oxford University Clinical Research Unit, Viet Nam and Prof. Shamala Devi, University of Malaya, Malaysia, respectively. DENV1-Westpac 74 (U88535), DENV1-05K2392 (Low et al., 2006), DENV2-TSV01 or DENV2-08K3115 were used for mouse experiments.

### —Cell Sorting and Sequence Analysis

Freshly isolated PBMCs were labeled with antibodies against CD20, CD27, CD19 (Biolegend) and CD138 (BD Pharmingen). For patient 10/50, DENV-specific memory B cells were identified based on their capacity of bind Alexa488-labelled DENV2-NGC (labeling kit from Molecular Probes). Cells were resuspended in sorting buffer (PBS, 2% FCS, 2 mM EDTA) for sorting into 96well PCR plates on a FACSAria (BD). The plates contained 10 mM Tris-HCL with 40 U/μl RNase inhibitor (Promega) and were immediately placed onto dry ice after sorting and stored at −80° C.

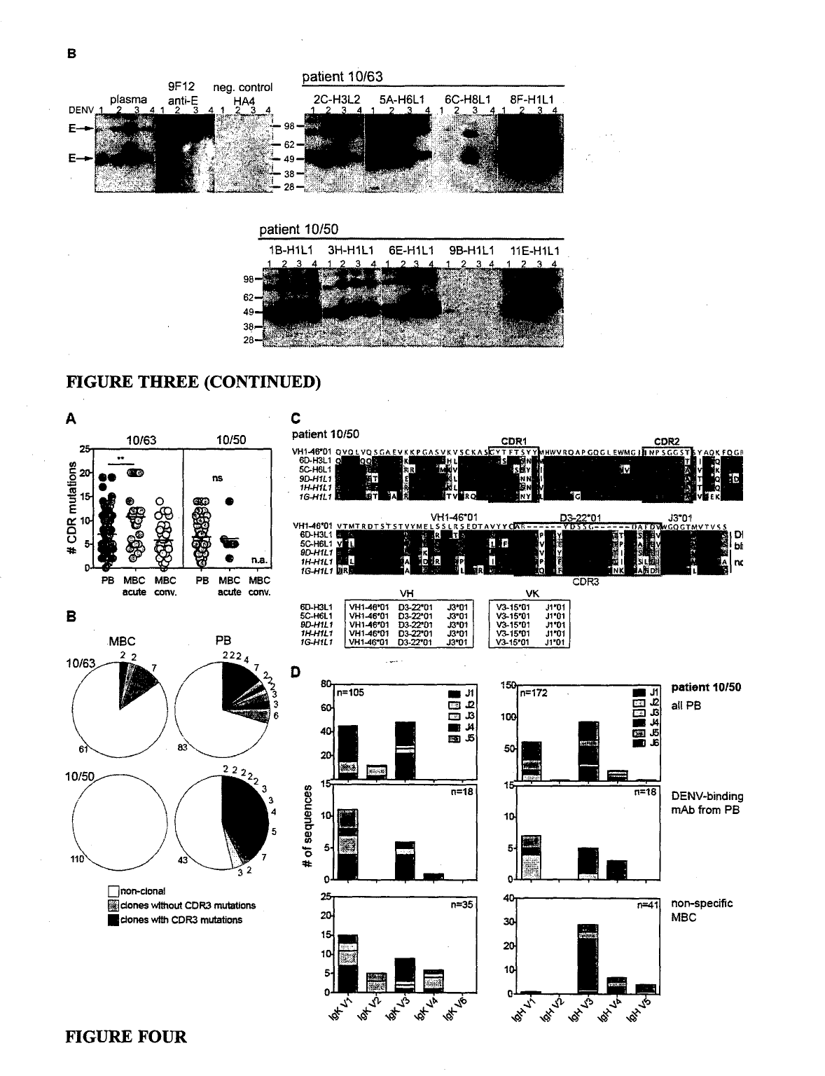

The mRNA of human IgG heavy and kappa light chains were amplified from single B cells by RT-PCR according to the protocol published by Smith et al. (Smith et al., 2009). The sequence quality was checked with Lasergene or CodonCodeAligner software. Sequences with high background noise or overlapping peaks were excluded from the analysis. vBase2 (www.vBase2.org) was used to determine the V and J gene family (FIG. 4). To calculate CDR3 length and mutations, sequences were analyzed with JOINSOLVER® (Souto-Carneiro et al., 2004). Germline sequences were from the IMGT database (Giudicelli et al. 2006).

### —Cloning, Expression and Purification of Antibodies

The mRNA of human IgG heavy and kappa light chains were amplified from single B cells by RT-PCR using the same protocol as specified in Smith et al. (Smith et al., 2009). For cloning into expression vectors RT-PCR products of selected B cells were used for nested PCR with modified primers: SalI and NheI sites were added at the 5′ and 3′ ends of the heavy chain and SalI and BbvCI sites were added at the 5′ and 3′ ends of the kappa light chain. The PCR products were cloned into the pTT5 mammalian expression vector (Durocher, et al. 2002) (licensed from the National Research Council Biotechnology Research Institute, Canada) using Infusion (Clontech) following the manufacturer's protocol. Plasmids containing productive sequences were subsequently used for recombinant antibody expression in HEK293-6E cells (Durocher et al., 2002). The control human IgG1 mAb HA4 (kindly provided by DSO National Laboratories, Singapore) is specific for H₅N₁ influenza virus, and human IgG1 mAb 5F-RC (Wrammert et al. 2012) is specific for chikungunya virus.

### —ELISA

For DENV-specific ELISA, MaxiSorp plates (Nunc) were coated with PEG-precipitated DENV serotypes 1-4. Plates were blocked with PBS, 0.05% Tween 20 (PBST) and 3% skimmed milk. Supernatants from Ab-expressing HEK cells were incubated on virus-coated plates for 1 h at RT before washing with PBST and detection of virus-binding antibodies with a secondary anti-human IgG-HRP (Sigma). For determination of absolute concentrations of IgG plates were coated with anti-Ig antibody (Caltag) and an IgG standard at different concentrations was included to generate a standard curve. EDIII-specific antibodies were measured on plates coated with EDIII of all four serotypes (ProSpec-Tany Technogene Ltd.) at a concentration of 150 ng per well. Purified antibodies were used for screening. E protein-specific antibodies were measured on plates coated with 300 ng/well E protein of DENV1, DENV3 (ProSpec-Tany Technogene Ltd.) or 150 ng/well E protein of DENV2-TSV01, which was produced in S2 cells as described in (Umashankar et al. 2008). Supernatants from Ab-expressing 293HEK cells or purified antibodies were used for screening. Pooled serum from several dengue-immune healthy donors was used as a positive control. 3,3,5,5-tetramethylbenzidine HRP substrate solution (TMB, Sigma) was used as substrate for all ELISAs. An OD value 2-fold higher than the background was defined as a positive signal.

### —Western Blot

Virus produced in C6/36 cells was PEG-precipitated and re-suspended in NTE buffer. Concentrated viral particles were heated at 95° C. for 5 min in non-reducing Laemmli buffer, prior to being separated by electrophoresis (NuPAGE 4-12% Bis-Tris Gel, Invitrogen) and electro-transferred onto PVDF membranes (Hybond-P, Amersham, GE Healthcare). The membranes were incubated with mAbs or a pool of plasma from dengue-immune healthy donors (1:20'000), followed by peroxidase-conjugated goat anti-human or anti-mouse IgG (JacksonImmunoResearch, 1:10'000).

### —Neutralization Assay

A flow cytometry-based neutralization assay was used with modifications (Kraus et al., 2007). BHK21 cell monolayers were grown in 96 well plates. Heat-inactivated plasma samples or protein G-purified monoclonal antibodies diluted in RPMI medium without FCS were incubated with DENY 1 (05K2916), 2 (TSV01), 3 (VN32/96) or 4 (My04 31580) at approximately MOI 1 for 1 h at 37° C. Plasma-virus mixtures were then transferred onto the BHK21 monolayers and incubated for 2 h at 37° C. before adding RPMI, 5% FCS. After an incubation time of 2-3 days, cells were stained intra-cellularly with antibodies against NS 1 and E protein and then acquired on an LSRII flow cytometer (Becton Dickinson). Data were analyzed using FlowJo software (TreeStar Inc.). The proportions of infected cells were plotted against the dilution factor and the EC50 was calculated with Prism5 (Graphpad Software) applying a three-parameter non-linear curve fit. Values with a curve fit of R²>0.95 were considered for the analysis.

### —Mouse Experiments

AG129 mice (U&K Universal, UK) were housed under SPF conditions at the Biological Resource Center (BRC), Singapore. 100 ug purified mAb in PBS was injected i.p., followed by virus infection via the same route 5-24 h later (3-6×10⁶ pfu/mouse). The animal experiments were conducted according to the rules and guidelines of the Agri-Food and Veterinary Authority (AVA) and the National Advisory Committee for Laboratory Animal Research (NACLAR), Singapore. The experiments were reviewed and approved by the Institutional review board of Biological Resource Center, Singapore (IACUC; protocol #090461).

### —Statistics

Data were analyzed with Prism Software (version 5 for Mac) and the statistical tests used were indicated in the figure legends. A p value of <0.05 were considered statistically significant.

### 1.2 Results

### —Plasmablasts Isolated During Secondary Infection are Dengue-Specific but Serotype Cross-Reactive In Vitro

Total plasmablasts were isolated from two separate dengue patients (10/63 and 10/50; Table 1) to characterize plasmablast-derived antibodies in infected individuals. The DENV immune status of these subjects was firstly established using a flow-cytometry based infection-neutralization assay (Kraus et al., 2007) (Table 1).

TABLE 1

| | | | Current | Pre-Existing | |
|---|---|---|---|---|---|
| Patient | Age | Clinical | Infection | Immunity | Time of |
| ID | (y) | Diagnosis | (RT-PCR) | (NT₅₀)^{a} | PB Sort^{b} |
| 10/63 | 41 | Dengue with | DENV2 | DENV1: 3197 | Day 4 |
| | | warning signs; | | DENV2: 2770 | |
| | | platelet nadir: | | DENV3: 678 | |
| | | 16 × 10³/μl | | DENV4: 624 | |
| 10/50 | 24 | Dengue with | DENV3 | DENV1: 1701 | Day 6 |
| | | warning signs; | | DENV2: 25763 | |
| | | platelet nadir: | | DENV3: 355 | |
| | | 22 × 10³/μl | | DENV4: 2693 | |

^{a}Neutralizing titers were determined 45 h (10/63) and 17 h (10/50) after the onset of fever. Bold text indicates assumed DENV serotype of previous infection.
^{b}Time point after onset of fever.

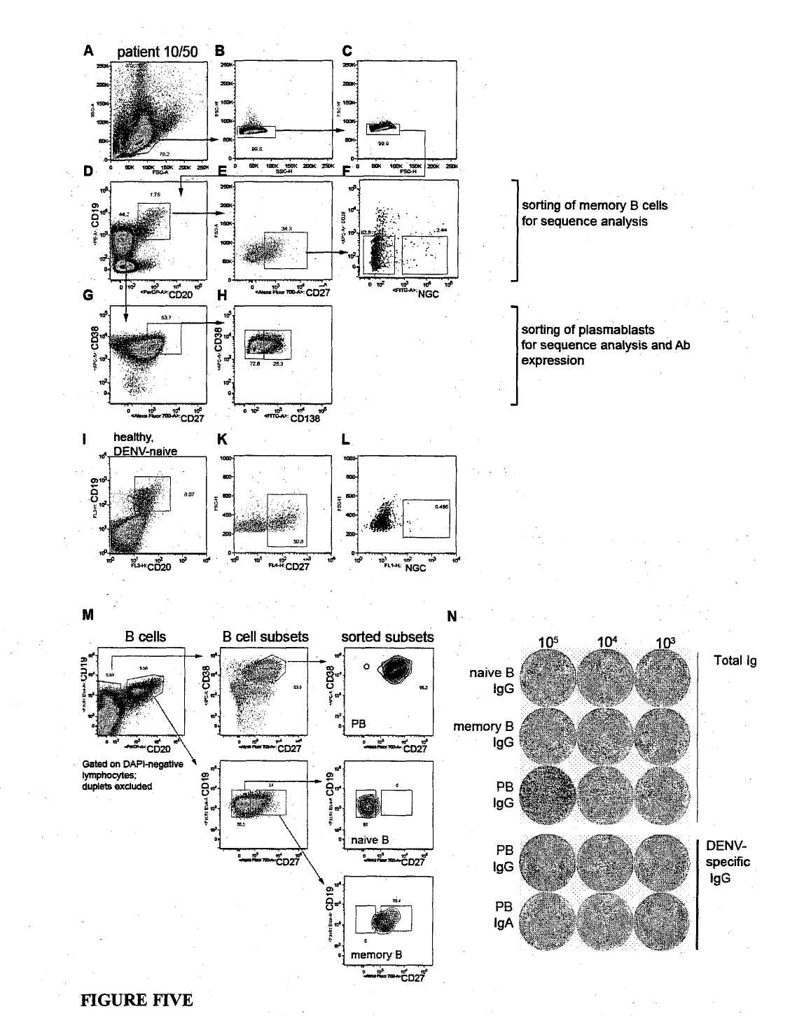

These data indicated that patient ID 10/63 had undergone a previous DENV1 infection, whereas patient ID10/50 had experienced a previous DENV2 and possibly a DENV4 infection. RT-PCR revealed that the viruses of the ongoing infection were serotypes DENV2 (ID10/63) and DENV3 (ID10/50). Plasmablasts were identified and sorted as CD20⁻CD19⁺CD27^{high} lymphocytes for patient 10/63 and as CD20⁻CD19⁺CD27^{high}CD138⁺ or CD138⁻ lymphocytes for patient 10/50. Antibody secretion by B cell subsets sorted from dengue-patients according to this strategy was verified by ELISPOT (FIG. 5). Plasmablasts were sorted into single wells of 96-well plates for nested RT-PCR to amplify the variable regions of the antibody heavy (VH) and light (VL) chains and for subsequent cloning into IgG1-expression vectors (Smith et al., 2009; Wrammert et al. 2008) (FIG. 1A). CD138⁺ plasmablasts were chosen from patient 10/50 for the expression of antibodies. IgG1 antibodies were tested in ELISA for their capacity to bind to viral particles (FIG. 1B; Table 2), which indicated that mAbs from patient 10/63 preferentially bound to DENV1 and 3, whereas mAbs from patient 10/50 bound preferentially to DENV2. Antibody binding to DENV1, 2, 3 or 4-infected BHK21 cells in fluorescent histology assays exhibited equal binding to all four virus serotypes for most clones (FIG. 1C; Table 2). A sequence alignment was generated of VH and VL regions from plasmablasts 10/63 FIG. 29A) and plasma cells 10/50 FIG. 29B) isolated at day 4 and 6, respectively, after onset of fever. CDR and FR are indicated.

In summary, 77% of the plasmablasts in patient 10/63 and 60% of the plasmablasts in patient 10/50 demonstrated binding activity to DENV by ELISA and/or in histology (FIG. 1D).

TABLE 2A
listing of the binding properties and neutralizing titers of all expressed antibodies (patient 10/63)
Patient 10/63

| ELISA/Histology | | | | | | Neutralization NT50 (ug/ml) | | | | | |
|---|---|---|---|---|---|---|---|---|---|---|---|
| | mAb ID | DENV1 | DENV2 | DENV3 | DENV4 | | mAb ID | DENV1 | DENV2 | DENV3 | DENV4 |
| 1 | 1A-H12L1 | | | | | 1 | 1A-H12L1 | | | | |
| 2 | 1B-H14L2 | | | E | | 2 | 1B-H14L2 | | | | |
| 3 | 1D-H4L1 | H | | E/H | | 3 | 1D-H4L1 | 1.35 | 0.23 | 0.04 | 2.55 |
| 4 | 1E-H3L1 | E | | | | 4 | 1E-H3L1 | | | | |
| 5 | 2A-H5L4 | | | E | | 5 | 2A-H5L4 | | | | |
| 6 | 2C-H3L2 | E/H | E | E/H | | 6 | 2C-H3L2 | | 0.31 | 0.24 | 0.90 |
| 7 | 2E-H2L2 | E | | | | 7 | 2E-H2L2 | | | | |
| 8 | 2F-H1L1 | E/H | E | E/H | E | 8 | 2F-H1L1 | 123.7 | 0.06 | 0.10 | 0.62 |
| 9 | 2G-H2L1 | E | | E | E | 9 | 2G-H2L1 | | | | |
| 10 | 3A-H6L1 | | | | | 10 | 3A-H6L1 | | | | |
| 11 | 3D-H2L2 | E/H | E | E | | 11 | 3D-H2L2 | | | | |
| 12 | 3E-H1L2 | E | | E | | 12 | 3E-H1L2 | | | | |
| 13 | 3F-H1L2 | E/H | | E | | 13 | 3F-H1L2 | | | | |
| 14 | 3H-H6L1 | | | E | | 14 | 3H-H6L1 | | | | |
| 15 | 4B-H1L2 | | | | | 15 | 4B-H1L2 | | | | |
| 16 | 4F-H6L1 | E/H | E | E/H | | 16 | 4F-H6L1 | 3.79 | 0.33 | 0.27 | >5 |
| 17 | 5A-H6L1 | E/H | E | E/H | | 17 | 5A-H6L1 | | 0.08 | 0.05 | |
| 18 | 5B-H1L1 | E/H | | | | 18 | 5B-H1L1 | | 0.21 | 0.14 | |
| 19 | 5C-H2L1 | | | H | | 19 | 5C-H2L1 | | | | |
| 20 | 5D-H1L2 | E/H | | H | | 20 | 5D-H1L2 | 0.38 | 0.13 | 0.01 | |
| 21 | 5G-H3L1 | | | | | 21 | 5G-H3L1 | | | | |
| 22 | 5H-H3L1 | E/H | | H | | 22 | 5H-H3L1 | | 0.05 | 0.05 | |
| 23 | 6A-H1L1 | | | | | 23 | 6A-H1L1 | | | | |
| 24 | 6C-H8L1 | E/H | | E/H | | 24 | 6C-H8L1 | 91.74 | 1.4 | 0.38 | |
| 25 | 6D-H5L2 | | | | | 25 | 6D-H5L2 | | | | |
| 26 | 6E-H6L1 | E/H | | E/H | | 26 | 6E-H6L1 | | 0.38 | 0.11 | |
| 27 | 7E-H1L1 | | | E/H | | 27 | 7E-H1L1 | | | | |
| 28 | 7F-H2L1 | | | | | 28 | 7F-H2L1 | | | | |
| 29 | 7G-H1L1 | E/H | E | E/H | | 29 | 7G-H1L1 | 9.67 | 3.23 | 0.14 | |
| 30 | 7H-H1L1 | | | | E | 30 | 7H-H1L1 | | | | |
| 31 | 8F-H1L1 | E/H | E | E/H | | 31 | 8F-H1L1 | | 0.01 | 0.06 | |

Bold: dengue-binding clones which were up-scaled and purified for neutralization assay
E and H denote clones which were positive in ELISA or Histology, respectively.

TABLE 2B
listing of the binding properties and neutralizing titers of all experessed antibodies (patient 10/50)
Patient 10/50

| ELISA/Histology | | | | | | | Neutralization NT50 (ng/ml) | | | | |
|---|---|---|---|---|---|---|---|---|---|---|---|
| | mAb ID | DENV1 | DENV2 | DENV3 | DENV4 | | mAb ID | DENV1 | DENV2 | DENV3 | DENV4 |
| 1 | 1B-H1L1 | E/H | E/H | E/H | E/H | 1 | 1B-H1L1 | | 0.036 | 0.006 | 0.025 |
| 2 | 1D-H8L1 | E/H | E/H | E/H | E/H | 2 | 1D-H8L1 | | 0.064 | 0.122 | |
| 3 | 1H-H1L1 | | | | | 3 | 1H-H1L1 | | | | |
| 4 | 2E-H1L1 | | | | | 4 | 2E-H1L1 | | | | |
| 5 | 2F-H1L3 | E/H | E/H | E/H | E/H | 5 | 2F-H1L3 | | 0.18 | 0.093 | |
| 6 | 3C-H5L1 | | | | | 6 | 3C-H5L1 | | | | |
| 7 | 3H-H1L1 | E/H | E/H | E/H | E/H | 7 | 3H-H1L1 | | 0.03 | 0.56 | 0.04 |
| 8 | 4B-H2L1 | E/H | E/H | E/H | H | 8 | 4B-H2L1 | 0.19 | | 0.019 | |
| 9 | 4E-H9L1 | | | | | 9 | 4E-H9L1 | | | | |
| 10 | 4F-H1L2 | | | | | 10 | 4F-H1L2 | | | | |
| 11 | 5A-H1L1 | E/H | E/H | E/H | H | 11 | 5A-H1L1 | | | | |
| 12 | 5C-H6L1 | | E/H | | | 12 | 5C-H6L1 | | | | |
| 13 | 5D-H6L2 | E/H | E/H | E/H | H | 13 | 5D-H6L2 | 0.064 | 0.09 | 0.144 | 0.039 |
| 14 | 6B-H1L2 | | E/H | E/H | | 14 | 6B-H1L2 | | | | |
| 15 | 6D-H3L1 | | E/H | E/H | | 15 | 6D-H3L1 | | | | |
| 16 | 6E-H1L1 | E/H | E/H | E/H | H | 16 | 6E-H1L1 | | | 0.008 | 0.02 |
| 17 | 6G-H1L1 | | | | | 17 | 6G-H1L1 | | | | |
| 18 | 7A-H1L1 | E/H | E/H | E/H | | 18 | 7A-H1L1 | | | | 1.63 |
| 19 | 7B-H1L2 | | | | | 19 | 7B-H1L2 | | | | |
| 20 | 7C-H3L1 | | E/H | | | 20 | 7C-H3L1 | | | | |
| 21 | 7E-H1L1 | E/H | E/H | E/H | E/H | 21 | 7E-H1L1 | | 0.24 | 0.14 | |
| 22 | 7H-H1L1 | E/H | E/H | E/H | | 22 | 7H-H1L1 | | | | |
| 23 | 8D-H9L1 | | | | | 23 | 8D-H9L1 | | | | |
| 24 | 8F-H1L2 | | | | | 24 | 8F-H1L2 | | | | |
| 25 | 9B-H1L1 | E/H | E/H | E/H | E/H | 25 | 9B-H1L1 | | | | |
| 26 | 9D-H1L1 | | | | | 26 | 9D-H2L2 | | | | |
| 27 | 9E-H2L2 | E/H | E/H | E/H | E/H | 27 | 9E-H2L2 | 0.04 | 0.044 | 0.167 | 0.021 |
| 28 | 10G-H1L11 | | | | E | 28 | 10G-H1L11 | | | | |
| 29 | 11D-H1L1 | | | | | 29 | 11D-H1L1 | | | | |
| 30 | 11E-H1L1 | E/H | E/H | E/H | H | 30 | 11E-H1L1 | | 0.18 | 0.041 | |

Bold: dengue-binding clones which were scaled up and purified for neutralization assay
E and H denote clones which were positive in ELISA or Histology, respectively.

### —The Original Serotype of Infection is Neutralized More Efficiently In Vivo

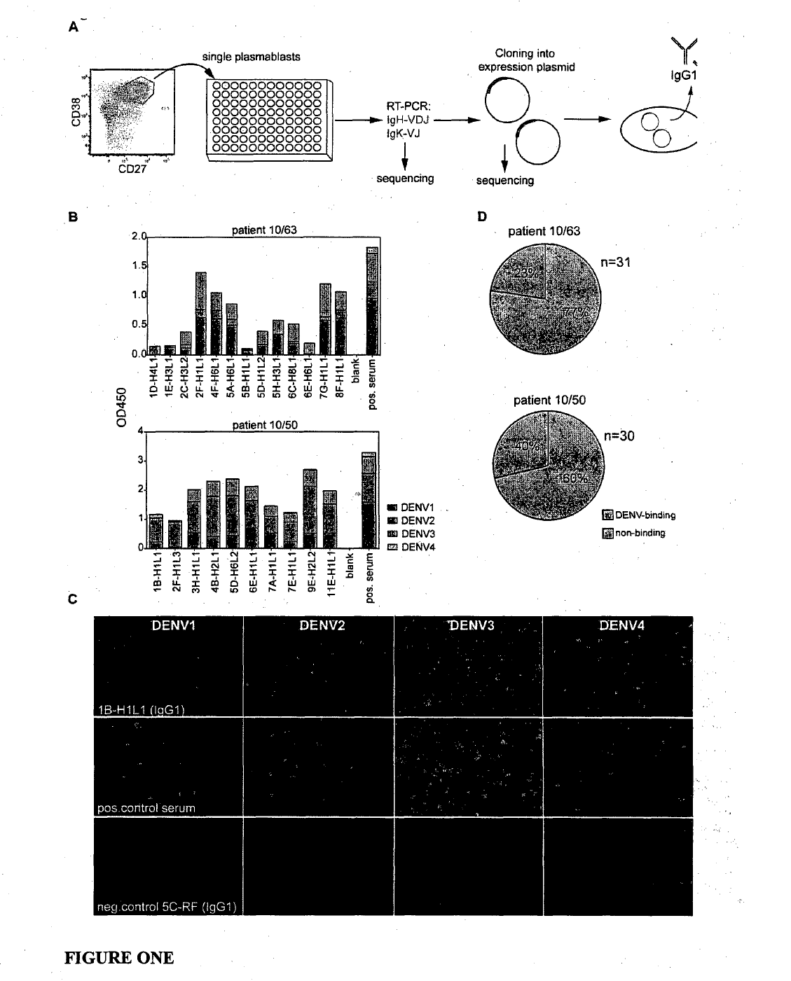

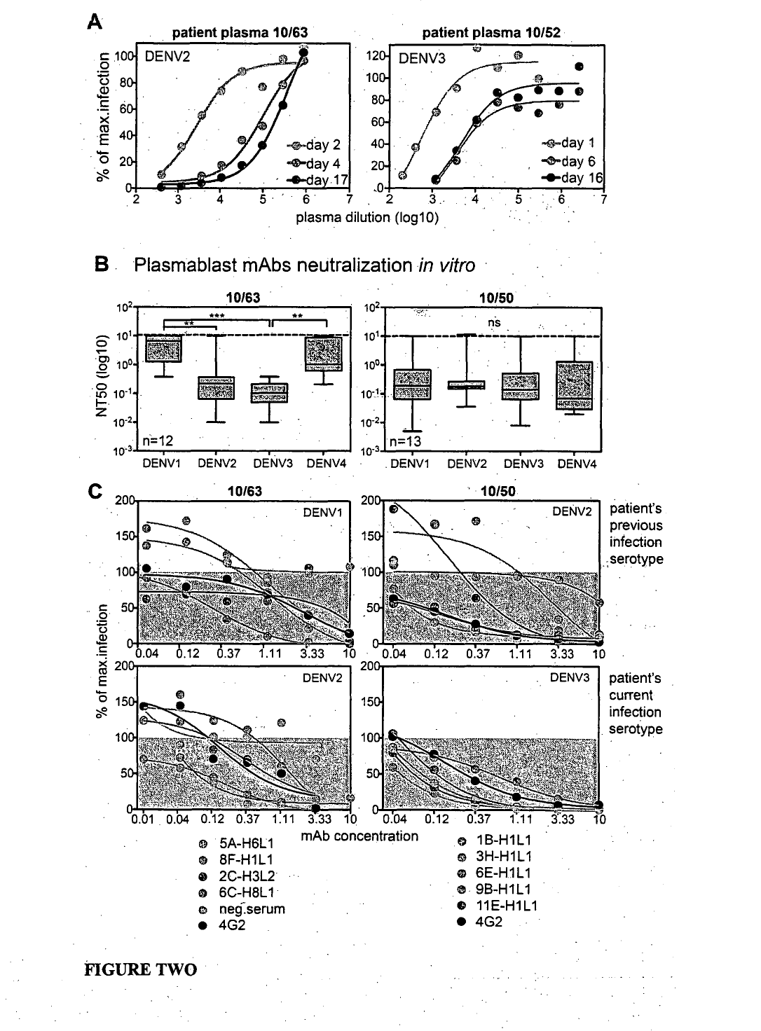

Patient plasma obtained during the time of plasmablast isolation (day 4 or 6, respectively) already showed efficient neutralization of the new serotype of infection in vitro (FIG. 2A). To study the neutralizing capacity of individual plasmablast-derived antibodies compared to polyclonal plasma, the clones from each donor that showed strongest binding in ELISA (FIG. 1B) were tested in a flow cytometry-based neutralization assay. The 50% neutralizing titer (NT50) for most clones occurred at concentrations between 0.01 and 1 μg/ml, and most clones neutralized all four serotypes (FIG. 2B; Table 2).

In vitro neutralization assays offer only limited predictive value for dengue protection since in vitro culture of the highly mobile dengue virus seems to expose poorly accessible viral epitopes, leading to increased antibody binding and enhanced virus neutralization. The protective capacity of selected mAbs in a mouse model of dengue infection (Schul et al., 2007) was tested to address the in vivo efficacy. The mAbs selected were chosen based on their high in vitro neutralizing capacity against previous and/or current serotypes of infection (FIG. 2C). 100 μg of each mAb was injected i.p. 5-24 h before infection with DENV1, 2 or 3 (FIG. 2D). In contrast to in vitro neutralization data (FIG. 2C) but in line with preexisting titers in the plasma (Table 1), mAbs derived from patient 10/63 were more protective against DENV1 and mAbs derived from patient 10/50 were more protective against DENV2, as shown by the reduction of viremia compared with the control mAb HA4 or 5F-RC (FIG. 2D). 10/50 antibodies also reduced DENV1 viremia 4-10 fold (data not shown). However, there was no protection against the current serotype of infection. In fact, patient 10/50-derived antibodies tended to increase DENV3 infection (FIG. 2D).

In summary, these data are consistent with the re-activation of cross-reactive memory B cells with a higher protecting capacity against the previous serotypes of infection.

### —Plasmablast Abs Bind to the Virus Surface E Protein

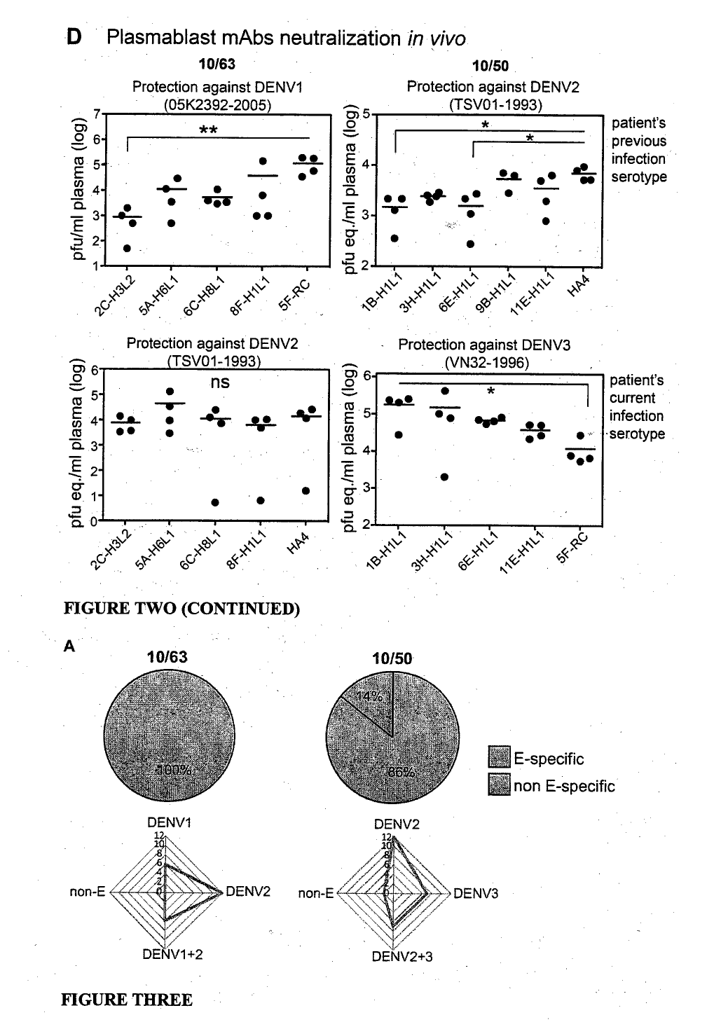

The virus particle ELISA used for the first antibody screen (FIG. 1A) showed that most antibodies bound to structural proteins that comprise the coat of virus particles. The virus coat of mature particles consists of the E protein and the M protein, whereby M is a transmembrane protein and not exposed on the surface. However, the un-cleaved form, called prM, is maximally exposed on immature virus particles, and virus preparations normally contain small amounts of immature particles. To confirm E protein binding ELISA plates were coated with recombinant E protein and found that 86-100% of the mAbs bound to the recombinant E protein serotype of either the current or previous infection (FIG. 3A; Table 3). The mAbs tested in mice were further assessed in Western Blot. mAbs bound to E protein monomers (ca. 48 kD) and dimers (ca. 100 kD) (FIG. 3B), and no binding to prM was observed except for the pooled human serum that was used as a positive control. All mAb clones were also tested for binding to EDIII, the E protein domain that contains the most effective neutralizing epitopes. However, none of the antibodies tested bound to EDIII, at least not to the soluble monomeric form used in these assays (Table 3).

In contrast to the memory B response, which is thought to be dominated by prM- and nonstructural protein-specific cells, plasmablasts preferentially bound to the E protein. This in turn implies that mature virus particles are the activating antigen that triggers B cells early during infection.

TABLE 3A
E protein binging of mAB (patient 10/63)
Patient 10/63

| | | EProtein ELISA | | |
|---|---|---|---|---|
| No. | mAb ID | DENV 1 | DENV2 | EDIII ELISA |
| 1 | 1B-H14L2 | − | | |
| 2 | 1D-H4L1 | − | + | − |
| 3 | 1E-H3L1 | − | | |
| 4 | 2A-H5L2 | − | | |
| 5 | 2C-H3L2 | − | + | − |
| 6 | 2E-H2L2 | − | | |
| 7 | 2F-H1L1 | + | + | − |
| 8 | 2G-H2L1 | − | | |
| 9 | 3D-H2L2 | − | | |
| 10 | 3E-H1L2 | − | | |
| 11 | 3F-H1L2 | − | | |
| 12 | 4F-H6L1 | + | + | − |
| 13 | 5A-H6L1 | + | + | − |
| 14 | 5B-H1L1 | − | + | − |
| 15 | 5C-H2L1 | | | |
| 16 | 5D-H1L2 | − | + | − |
| 17 | 5G-H3L1 | − | | |
| 18 | 5H-H3L1 | + | + | − |
| 19 | 6C-H8L1 | − | + | − |
| 20 | 6E-H6L1 | − | + | − |
| 21 | 7E-H1L1 | − | | |
| 22 | 7G-H1L1 | + | + | − |
| 23 | 7H-H1L1 | − | | |
| 24 | 8F-H1L1 | + | + | − |
| | Summary of | 26% (6/23) | 100% (12/12) | 0% (0/12) |
| | binding^{a} | | | |

TABLE 3B
E protein binging of mAB (patient 10/50)
Patient 10/50

| | | EProtein ELISA | | |
|---|---|---|---|---|
| No. | mAb ID | DENV2 | DENV3 | EDIII ELISA |
| 1 | 1B-H1L1 | + | + | − |
| 2 | 3H-H1L1 | + | + | − |
| 3 | 5A-H1L1 | − | − | |
| 4 | 6E-H1L1 | + | + | − |
| 5 | 7A-H1L1 | + | − | − |
| 6 | 7H-H1L1 | + | − | |
| 7 | 9B-H1L1 | − | − | − |
| 8 | 11E-H1L1 | + | + | − |
| 9 | 1D-H8L1 | + | − | |
| 10 | 2F-H1L3 | + | − | − |
| 11 | 4B-H2L1 | + | + | − |
| 12 | 5C-H6L1 | + | | |
| 13 | 5D-H6L2 | + | + | − |
| 14 | 6B-H1L2 | + | | |
| 15 | 6D-H3L1 | + | | |
| 16 | 7C-H3L1 | + | | |
| 17 | 7E-H1L1 | + | − | − |
| 18 | 9E-H2L2 | + | + | − |
| 19 | 10G-H1L11 | − | | |
| | Summary of | 84% (16/19) | 50% (7/14) | 0% (0/11) |
| | binding^{a} | | | |

Clones in bold type were tested further in neutralization assays (Supplemental Table I).
^{a}Number of binding clones out of total clones tested; only those clones with “−” or “+” were tested in the respective assay.

—The Plasmablast Response is Polyclonal and Generated from Affinity-Matured and Selected Memory B Cells Based on Sequence Analysis

Plasmablasts isolated in the current report were class-switched (IgG) and were thus likely to have undergone selection in a germinal center. Given that DENV is an acute infection, it was hypothesized that the B cell clones studied in the current report may have undergone limited affinity maturation and may therefore have maintained the capacity to partially neutralize several serotypes. To address this question, the affinity maturation of plasmablasts relative to that of non-specific “general” memory B cells was compared based on numbers of CDR mutations. The MBCs isolated from patient 10/50 were CD19⁺CD27⁺ and non-dengue-specific, selected based on their inability to bind to fluorescently labeled DENV2 virus, whereas cells from patient 10/63 were collected from the total pool of CD19⁺CD27⁺ MBCs and might thus contain dengue-specific memory cells besides the larger pool of non-specific memory cells. PCR products from single cells were sequenced for this analysis (see scheme in FIG. 1A). In the two patients analyzed here the number of CDR mutations was similar in plasmablasts and memory B cells (FIG. 4A). It was next tested whether few high affine clones were expanded and dominated the PB response, as observed after influenza infection. FIG. 4B illustrates that while clonal expansion was observed particularly for the plasmablast population the response remained polyclonal. Interestingly, amino acid mutations at the junction and CDR3 region were observed for many clones. Amongst the PB sequences chosen for recombinant Ab expression, five mAbs from patient 10/50 used the same heavy and light chain V(D)J elements and thus appeared to originate from the same progenitor cell. However, the introduction of mutations led to the loss of dengue specificity in three of the five plasmablasts (FIG. 4C).

In summary, plasmablasts were generated from a very diverse, affinity-matured and selected pool of memory B cells that did not proliferate extensively before differentiating into plasmablasts. However, limited proliferation might allow for the introduction of mutations for further repertoire diversification, which can also lead to the loss of dengue-specificity.

—Dengue-Specific Plasmablasts Use Unique VDJ Combinations Compared with Memory B Cells

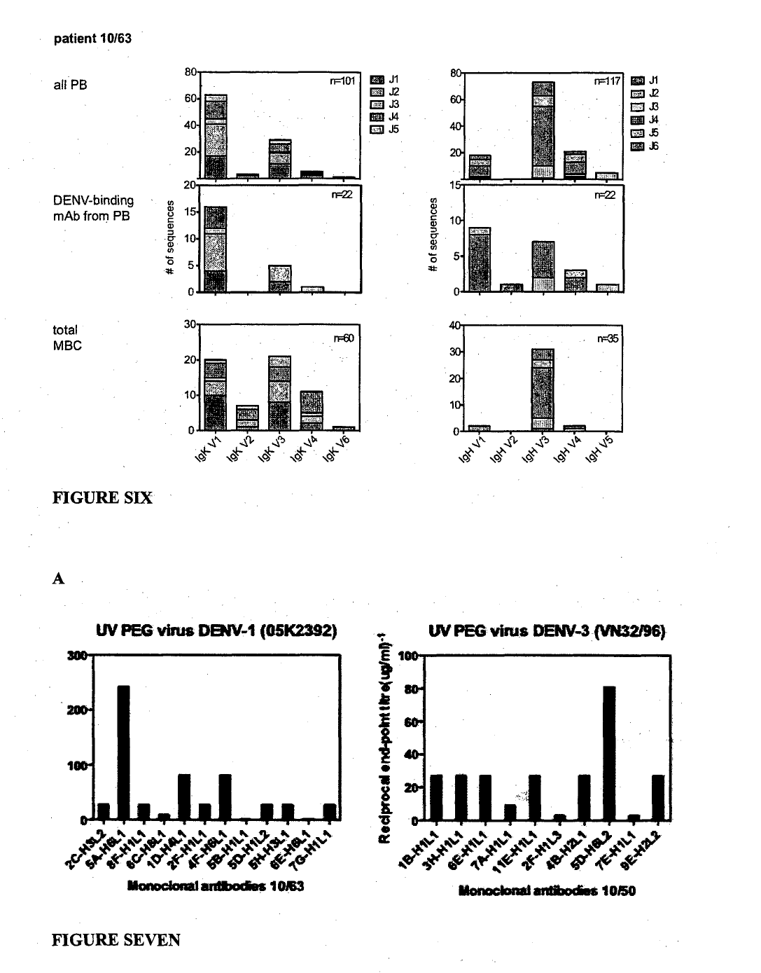

To address a potential dengue-specific genetic pattern the V and J gene family usage of both heavy and light chains in dengue-specific and non-specific B cell populations of the multi-serotype-specific plasmablasts isolated here was determined (FIG. 4D). Sequences from all plasmablasts were compared with sequences from confirmed DENV-binding plasmablasts and with sequences from non-specific memory B cells that were isolated as described in the previous paragraph. For both patients 10/63 (FIG. 6) and 10/50 (FIG. 4D) the VDJ gene family usage of plasmablasts was restricted compared with the broader VDJ gene family usage in non-specific “general” memory B cells, and the unique VDJ usage in confirmed dengue-binding plasmablasts (expression of recombinant mAbs; FIG. 1A) was even more pronounced than in total plasmablasts. The repertoire of CD138⁺ and CD138⁻ plasmablasts was similar for patient 10/50 (data not shown).

Interestingly, dengue-binding plasmablasts from both patients showed a preference for VH1 family gene usage, whereas VH3 gene usage was dominant in MBCs. VH3 normally dominates total peripheral B cells in healthy individuals. These data suggest that dengue virus selectively binds to B cells using rare V family genes, which are efficiently activated and differentiate into plasmablasts during acute disease.

### 1.3 Discussion

During acute disease, pre-existing antibodies in the circulation produced by long-lived plasma cells are present alongside antibodies produced from newly activated plasmablasts. Since the plasmablast response in dengue patients is fulminant and likely has an impact on the disease, the aim of the current study was to address the antigen-specificity of the total plasmablast response and to identify the specificity and protective capacity of individual cells. Moreover, since many symptomatic dengue infections are secondary cases and with the prospect of a dengue vaccine becoming available in the near future the question was asked whether the plasmablast response in secondary patients originated from specific memory and/or naïve B cells. Patient-derived plasmablasts were therefore sorted during acute-phase disease and the mAbs they expressed were analyzed. Plasmablasts were found to be predominantly dengue virus-specific in the patients studied here (respectively 60% and 77% specificity). Plasmablasts comprising mostly re-activated memory B cells are thus dominating the secondary acute response, accounting for the original antigenic sin phenomenon. At the same time a primary response to the new serotype of infection is initiated, eventually leading to immunity against past and current serotypes of infection.

On the individual cell level, it was observed that E-protein-specific cells dominated the acute plasmablast response. This is in contrast to the previously reported memory B cell repertoire in dengue patients where 50% mAbs bind to NS1, NS3, prM or capsid. All antibodies that bound to infected BHK-21 cells in histology also bound to virus particles in ELISA (Table 2). The virus particle preparations used for ELISA did not contain NS 1 (the plasma control containing anti-NS 1 antibodies did not show a band typical for NS1 in the western blot in FIG. 3 for which the same virus particles were used) and binding to NS1 was therefore excluded. However, binding to NS- and prM proteins was not specifically tested and not all antibodies binding in the virus particle ELISA were available for the recombinant E protein ELISA (Table 3).

The E protein is exposed on the virus coat and contains neutralizing Ab epitopes. E protein-specificity of the presently disclosed Abs can therefore explain the serotype-specific partial protection observed in animal experiments (FIG. 2C). However, the Ab binding sites seemed to include highly conserved amino acids since most antibodies bound to all serotypes in vitro. It fact, DENV neutralizing Ab epitopes may consist of quarternary structures, spanning over two adjacent E protein dimers and involving E domains I and II or III, respectively suggesting that Ab binding may involve both serotype-specific and conserved epitopes. Despite binding activity of the plasmablast mAbs in Western Blot, which indicated that the presently disclosed antibodies recognize linear epitopes binding to peptides was not observed (15mers with 5AA overlap).

A key question besides the specificity of Abs is the biological origin of the B cells secreting them. Cross-reactive antibodies prevail after both a primary and secondary infection and it is therefore not clear which B cells are activated during a secondary heterologous infection. Knowledge about the nature of activated or re-activated cells is of particular relevance in the context of vaccination.

Plasmablast Ig sequences identified after influenza infection were highly mutated, exceeding the mutation rate of memory B cells. Here it was observed that dengue infection generated plasmablasts that showed a mutation rate similar to memory cells in the same patients. Moreover, plasmablasts had undergone selection based on replacement versus silent mutations in framework and CDR regions (data not shown), further demonstrating their memory B cell origin. Preferential activation of B cells with the highest affinities has been demonstrated after influenza vaccination in humans and in mouse models, suggesting that high affinity of a memory cell is a general prerequisite for the initiation of plasmablast differentiation during acute disease. At least for the two patients analyzed here, plasmablast responses were relatively polyclonal compared to the pauci-clonal response in influenza vaccinees with pre-existing immunity.

Strikingly, dengue-specific B cells used VH and VL gene families with a bias towards VH1 and Vkappa1 (FIG. 4D). VH1 gene bias might be representative of a B cell population activated in the context of viral infections. BCRs using VH1 gene families may be particularly suited to bind to virus coat glycoproteins and might reside in a lymphatic environment where the virus is accumulated or filtered.

In conclusion, secondary infections with a heterologous virus serotype engage pre-existing dengue-specific memory B cells from previous dengue infections and activate non-specific memory B cells to a lesser extent. The E protein specificity and diversity of plasmablast-derived antibodies may help clear the mutating virus faster after secondary than after primary infection. It seems unlikely that these antibodies have an impact on severity by enhancing infection because the plasmablast response peaks when viremia is already declining. It will be important to see to which extent plasmablast clones are selected into the memory pool and whether they change their specificity or affinity in the memory phase, as this could help to determine correlates of protection.

### Example 2

### Diagnostic and Treatment Applications of Anti-DENV Antibodies

### 2.1 Summary

The binding patterns of the antibodies described herein are summarised in the Examples above and accompanying Figures/Tables. Additional analyses were performed, as detailed below.

In particular, it was found that several human monoclonal antibodies (humAbs) bind to the whole virus particles, but much less or not at all to recombinantly expressed E protein, which is the major component of the virus structure. This represents an important feature for diagnostic applications.

Furthermore, novel bi-specific antibodies were generated by combining variable domains (Fab) of two different antibodies into “artificial” antibodies (Kontermann, 2012). Bi-specific antibodies were tested in dengue neutralization assays and showed a high neutralizing capacity that was superior to the one of the parent antibody.

### 2.2 Diagnostic Applications

### Preferential Binding of Antibodies to Whole Virus Particles

The antibodies described herein bind to the dengue virus E protein, the major component of the virus particle.

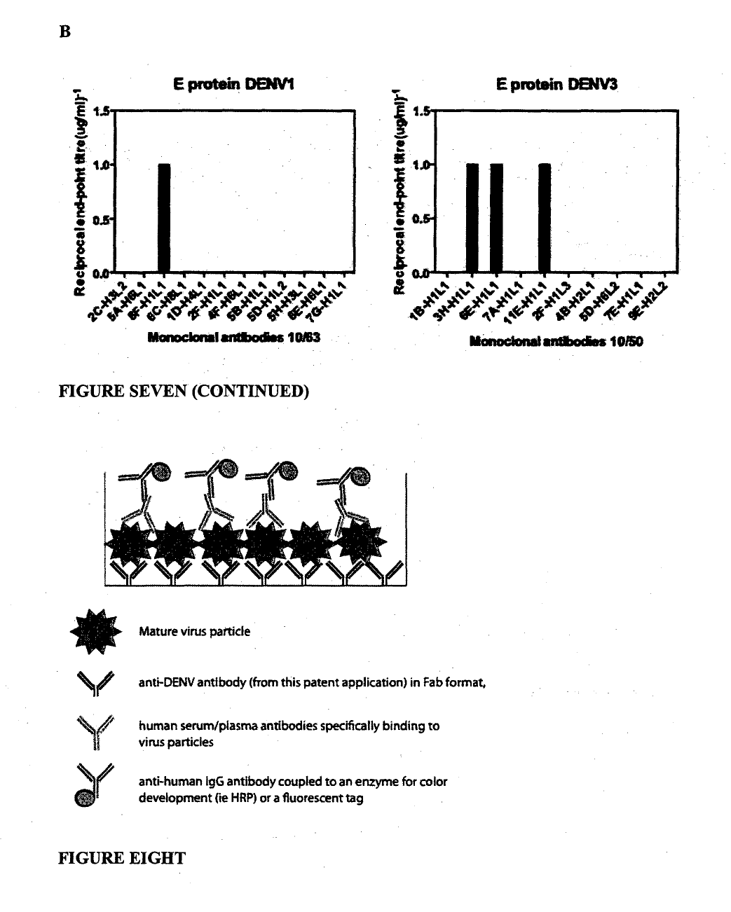

Antibodies that preferentially bind to the intact virus particle show a high affinity in ELISA (FIG. 7) and efficacy in vivo to decrease viremia (see Table 1 in Example 1 above). The epitopes of these antibodies likely comprise of a quarternary structure that is only present on intact, infectious virus particles. Such antibodies can be particularly useful for the detection of intact, infectious virus particles when used in bio-sensors to detect and quantify virus for diagnostic purpose or to detect and quantify virus in the course of attenuated vaccine production. Virus particle fragments and non-infectious immature virus particles, which comprise a major part of the viral material in human blood, can be excluded when employing an antibody that only binds to intact mature virus particles. Immature virus particles are coated by a protein called prM that shields the E protein, making it much less accessible to E-protein-specific antibodies. In addition to the potential application in biosensors, mature virus-specific antibodies can be used in ELISA-based assays to detect virus-specific antibodies for diagnostic purpose or to assess the specific antibody titers in vaccinated individuals in vaccine trials.

Exemplary antibodies described herein particularly useful for diagnostic purposes due to their high affinity to virus particles are summarized in Table 4. Exemplary applications are described in the following paragraphs.

TABLE 4
Summary of antibodies that are serotype specific and/or
show potent binding to intact virus particles

| | Patient ID | Serotype-specificity | Epitope |
|---|---|---|---|
| 5D-H1L2 | 10/63 | DENV2 >> DENV1/3/4 | E protein and whole |
| | | | virus particle |
| 2C-H3L2 | 10/63 | DENV1 > DENV2/3/4 | Whole virus particle |
| | | | >> E protein |
| 6C-H8L1 | 10/63 | DENV3 >> DENV1 > 2 | E protein and whole |
| | | | virus particle |
| 8F-H1L1 | 10/63 | DENV1 > DENV2/3/4 | E protein and whole |
| | | | virus particle |
| 5A-H6L1 | 10/63 | DENV1 >> DENV2/3/4 | Whole virus particle |
| | | | >> E protein |
| 11E-H1L1 | 10/50 | DENV2 > DENV1/3/4 | E protein and whole |
| | | | virus particle |
| 5D-H6L2 | 10/50 | DENV1/2/3/4 | Whole virus particle |
| | | | >> E protein |
| 6E-H1L1 | 10/50 | DENV2 > DENV1 > | E protein and whole |
| | | DENV3/4 | virus particle |

Quarternary structures on the whole virus particle are immunogenic, and three antibodies so far have been described in the literature to bind to viral structures that comprise of two adjacent E proteins in the arrangement that exists on mature virus particles (quarternary structures). Two of those antibodies, one specific to West Nile Virus (Thompson et al. 2009) and one specific for dengue (Teo et al., 2012) have been suggested as candidates for therapeutic antibodies.

The E protein is arranged in a dimeric or trimeric form on intact virus particles. The trimeric form is assumed by the virus before fusion with the endosome after uptake into host cells. The three antibodies mentioned in the paragraph above bind to the dimeric form that is found on mature virus particles.

In dengue diagnostics, the neutralization assay is the gold standard to assess protective antibody titers. However, due to the inherent biophysical characteristics of dengue virus it is almost impossible to standardize the neutralization assay (Dowd et al. 2011; WHO, 2009). The neutralization assay therefore offers limited value and additional diagnostic tools are needed (Sabchareon et al. 2012).

ELISA-based assays represent an alternative to assess potentially protective antibody titers in patients or vaccinees. However, it has not been established which antibodies, when employed in ELISA, result in the best correlation with neutralizing titers. The antibodies described herein are effective for use as a “neutralization assay correlate” due to their efficient binding to virus particles.

### —Exemplary Application: Biosensors to Detect Mature Virus Particles

Biosensors to detect virus particles are well established, and several of the described biosensors make use of antibodies to detect specific virus particles. The antibodies described herein could be tested in biosensors. For the detection of individual dengue serotypes, serotype-specific antibodies would need to be used for binding to the sensor.

### —Exemplary Application: ELISA-Based Technologies to Detect Dengue-Specific Antibodies

### (i) Capture ELISA

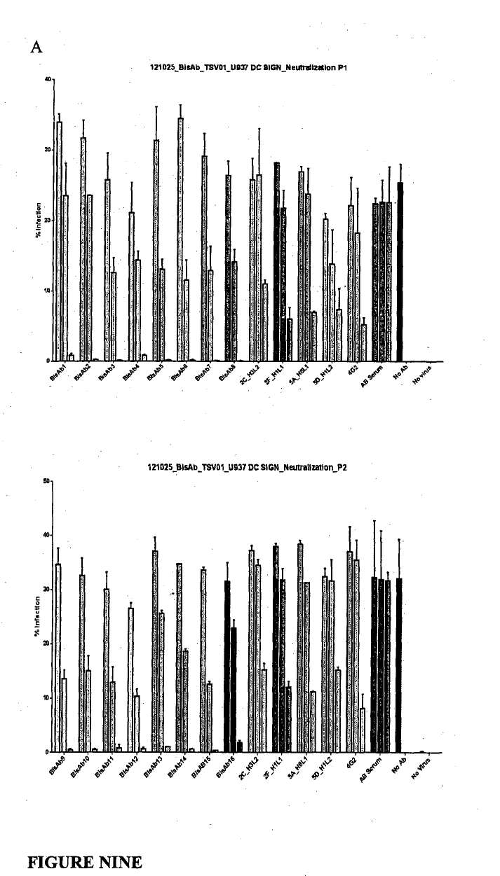

The antibodies described herein can be used for the diagnostic detection of DENV-specific antibodies in patients or in vaccinees. An exemplary format of ELISA-based detection of DENV-specific antibodies is illustrated in FIG. 8. Several other formats such as beads instead of ELISA plates etc. are possible to apply the same principle, namely the detection of Abs specific for intact virus particle with the help of the DENV-specific Abs described herein.

### (ii) Competition ELISA

Based on the additional analysis (Table 4; FIG. 7), the use of whole virus particles for coating of ELISA plates or beads is suggested as an alternative to coating with EDIII or E protein. The DENV-specific Abs described herein bind with high affinity and compete with cross-reactive or heterologous serotype-specific antibodies of lower affinity in the serum of patients (see Examples above). This can applied for two readouts:

A. Distinguish Between Primary and Secondary Infection:

Abs with a high affinity for conserved epitopes are only present in patients with a secondary infection. Competition between unlabelled patient plasma and a tagged anti-DENV Ab from this patent indicates a previous infection (see FIG. 5 in the patent draft). Knowledge about pre-existing dengue infections is of relevance for clinicians to decide whether a patient is at higher risk to develop a severe disease

B. Determine the Serotype-Specific Component of Abs to Test Balanced Immune Responses in Vaccines:

The antibodies with a preferential serotype-specific binding in animal experiments (See Example 2 above) or in ELISA (FIG. 7) can be employed to detect whether a patient's antibody repertoire covers all four serotypes. Competition of serum with serotype-specific antibodies will indicate the presence of serum antibodies specific for the respective serotype.

### 2.3 Therapeutic Applications

Bi-specific antibodies were generated by linking the variable domain of a given antibody with the variable domain of a different antibody via a protein linker. The bi-specific antibodies were expressed in HEK cells and tested for their capacity to neutralize dengue infection in a fluorescene-based neutralization assay (Xu et al, J Immunol 2012).

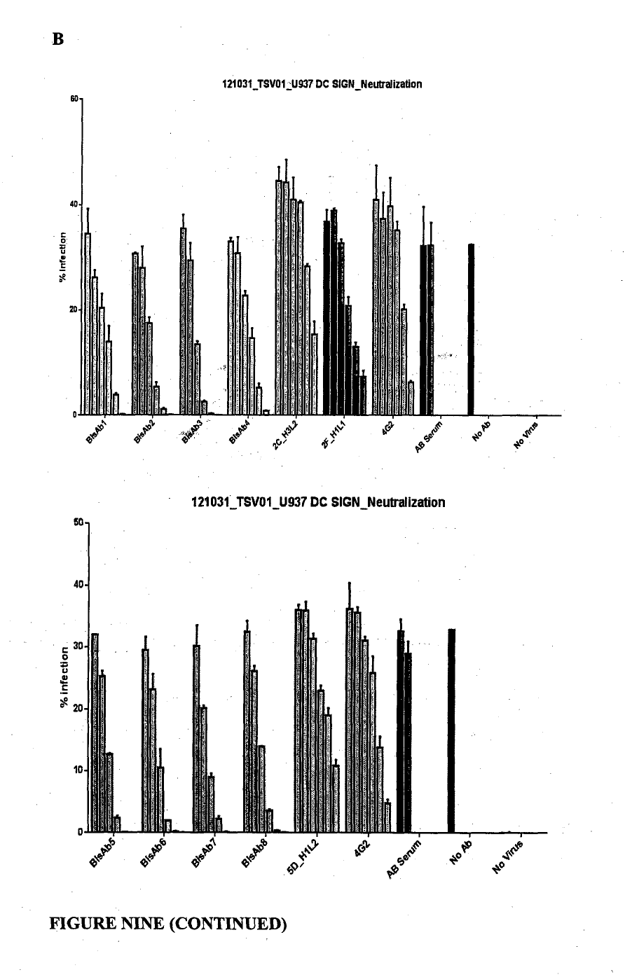

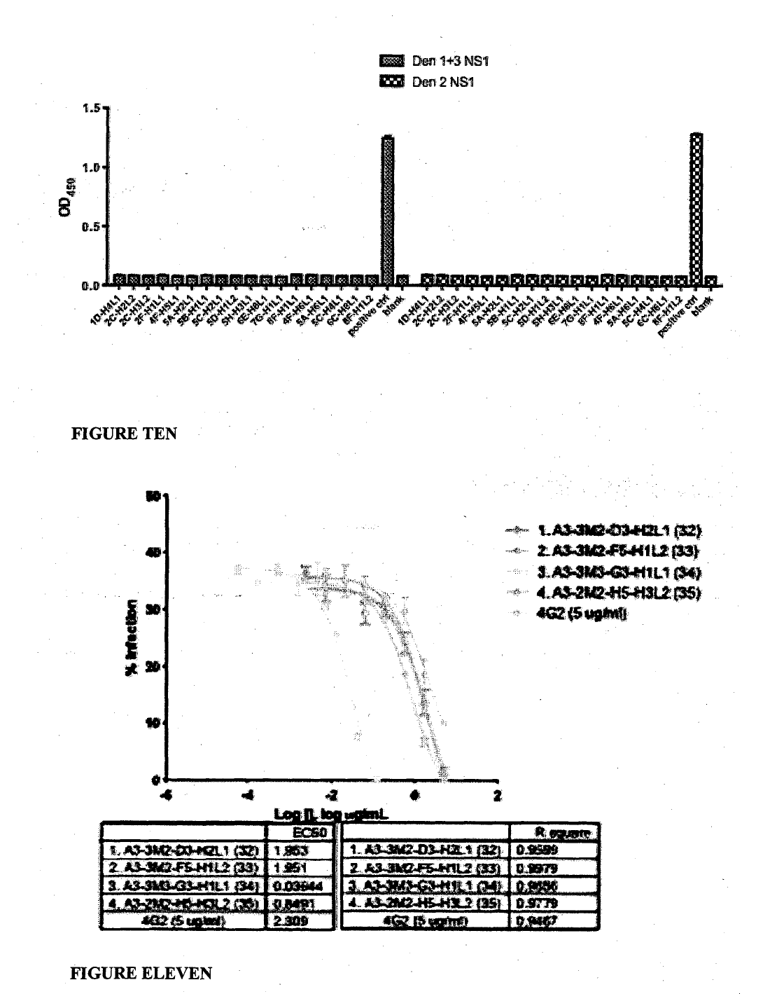

The antibody combinations 1-16 in Table 2 were tested in neutralization assay as shown in FIG. 9.

TABLE 5
Antibody combinations for testing in neutralization assay
as shown in FIG. 9

| | 2C_H3L2 | 2F_H1L1 | 5A-H6L1 | 5D_H1L2 |
|---|---|---|---|---|
| 2C_H3L2 | 1 | 2 | 3 | 4 |
| 2F_H1L1 | 5 | 6 | 7 | 8 |
| 5A-H6L1 | 9 | 10 | 11 | 12 |
| 5D_H1L2 | 13 | 14 | 15 | 16 |

Abs in green boxes indicate mono-specificity bi-specific Abs

The neutralization data are shown in FIG. 9. The neutralizing capacity of dual-specificity bi-specific antibodies is higher than the capacity of the parent antibodies. Moreover, the neutralizing capacity of mono-specificity bi-specific antibodies is higher than the parent antibody.

In addition to the combinations shown in Table 5, additional combinations will be tested for their neutralizing capacity against one or more dengue serotypes. The combinations can comprise of any of the antibodies originally described in the patent application or they can comprise of combinations between newly identified antibodies. All possible combinations are indicated in Table 6.

TABLE 6

| combinations of bi-specific antibodies with potential to bind and neutralize dengue virus. | | | | | | | | | | | | | | | | |
|---|---|---|---|---|---|---|---|---|---|---|---|---|---|---|---|---|
| | ID-H4L1 | 1E-H3L1 | 2A-H5L4 | 2C-H3L2 | 2E-H2L2 | 2F-H1L1 | 2G-H2L1 | 3D-H2L2 | 3E-H1L2 | 3F-H1L2 | 4F-H6L1 | 5A-H6L1 | 5B-H1L1 | 5C-H2L1 | 5D-H1L2 | 5H-H3L1 |
| 1D-H4L1 | | | | | | | | | | | | | | | | |
| 1E-H3L1 | | | | | | | | | | | | | | | | |
| 2A-H5L4 | | | | | | | | | | | | | | | | |
| 2C-H3L2 | | | | | | | | | | | | | | | | |
| 2E-H2L2 | | | | | | | | | | | | | | | | |
| 2F-H1L1 | | | | | | | | | | | | | | | | |
| 2G-H2L1 | | | | | | | | | | | | | | | | |
| 3D-H2L2 | | | | | | | | | | | | | | | | |
| 3E-H1L2 | | | | | | | | | | | | | | | | |
| 3F-H1L2 | | | | | | | | | | | | | | | | |
| 4F-H6L1 | | | | | | | | | | | | | | | | |
| 5A-H6L1 | | | | | | | | | | | | | | | | |
| 5B-H1L1 | | | | | | | | | | | | | | | | |
| 5C-H2L1 | | | | | | | | | | | | | | | | |
| 5D-H1L2 | | | | | | | | | | | | | | | | |
| 5H-H3L1 | | | | | | | | | | | | | | | | |
| 6C-H8L1 | | | | | | | | | | | | | | | | |
| 6E-H6L1 | | | | | | | | | | | | | | | | |
| 7E-H1L1 | | | | | | | | | | | | | | | | |
| 7G-H1L1 | | | | | | | | | | | | | | | | |
| 7H-H1L1 | | | | | | | | | | | | | | | | |
| 8F-H1L1 | | | | | | | | | | | | | | | | |
| 1B-H1L1 | | | | | | | | | | | | | | | | |
| 1D-H8L1 | | | | | | | | | | | | | | | | |
| 2F-H1L3 | | | | | | | | | | | | | | | | |
| 3H-H1L1 | | | | | | | | | | | | | | | | |
| 4B-H2L1 | | | | | | | | | | | | | | | | |
| 5A-H1L1 | | | | | | | | | | | | | | | | |
| 5C-H6L1 | | | | | | | | | | | | | | | | |
| 5D-H6L2 | | | | | | | | | | | | | | | | |
| 6B-H1L2 | | | | | | | | | | | | | | | | |
| 6D-H3L1 | | | | | | | | | | | | | | | | |
| 6E-H1L1 | | | | | | | | | | | | | | | | |
| 7A-H1L1 | | | | | | | | | | | | | | | | |
| 7C-H3L1 | | | | | | | | | | | | | | | | |
| 7E-H1L1 | | | | | | | | | | | | | | | | |
| 7H-H1L1 | | | | | | | | | | | | | | | | |
| 9H-H1L1 | | | | | | | | | | | | | | | | |
| 9E-H2L2 | | | | | | | | | | | | | | | | |
| 11E-H1L1 | | | | | | | | | | | | | | | | |
| A3-3M1-B11-H1L2 | | | | | | | | | | | | | | | | |
| A3-3M1-D1-H1L3 | | | | | | | | | | | | | | | | |
| A3-3M2-D3-H2L1 | | | | | | | | | | | | | | | | |
| A3-3M2-F5-H1L2 | | | | | | | | | | | | | | | | |
| A3-3M3-C4-H3L3 | | | | | | | | | | | | | | | | |
| A3-3M3-G3-H1L1 | | | | | | | | | | | | | | | | |
| A3-2M1-A5-H1L1 | | | | | | | | | | | | | | | | |
| A3-2M2-G8-H1L2 | | | | | | | | | | | | | | | | |
| A3-2M2-H5-H3L2 | | | | | | | | | | | | | | | | |
| A3-2M3-C9-H2L1 | | | | | | | | | | | | | | | | |
| A3-2M3-E9-H1L2 | | | | | | | | | | | | | | | | |
| A3-2M3-F9-H2L1 | | | | | | | | | | | | | | | | |
| A3-2M3-G3-H2L3 | | | | | | | | | | | | | | | | |
| A3-3M3-C2-H3L1 | | | | | | | | | | | | | | | | |
| | 6C-H8L1 | 6E-H6L1 | 7E-H1L1 | 7G-H1L1 | 7H-H1L1 | 8F-H1L1 | 1B-H1L1 | 1D-H8L1 | 2F-H1L3 | 3H-H1L1 | 4B-H2L1 | 5A-H1L1 | 5C-H6L1 | 5D-H6L2 | 6B-H1L2 | 6D-H3L1 |
| 1D-H4L1 | | | | | | | | | | | | | | | | |
| 1E-H3L1 | | | | | | | | | | | | | | | | |
| 2A-H5L4 | | | | | | | | | | | | | | | | |
| 2C-H3L2 | | | | | | | | | | | | | | | | |
| 2E-H2L2 | | | | | | | | | | | | | | | | |
| 2F-H1L1 | | | | | | | | | | | | | | | | |
| 2G-H2L1 | | | | | | | | | | | | | | | | |
| 3D-H1L2 | | | | | | | | | | | | | | | | |
| 3E-H1L2 | | | | | | | | | | | | | | | | |
| 3F-H1L2 | | | | | | | | | | | | | | | | |
| 4F-H6L1 | | | | | | | | | | | | | | | | |
| 5A-H6L1 | | | | | | | | | | | | | | | | |
| 5B-H1L1 | | | | | | | | | | | | | | | | |
| 5C-H2L1 | | | | | | | | | | | | | | | | |
| 5D-H1L2 | | | | | | | | | | | | | | | | |
| 5H-H3L1 | | | | | | | | | | | | | | | | |
| 6C-H8L1 | | | | | | | | | | | | | | | | |
| 6E-H6L1 | | | | | | | | | | | | | | | | |
| 7E-H1L1 | | | | | | | | | | | | | | | | |
| 7G-H1L1 | | | | | | | | | | | | | | | | |
| 7H-H1L1 | | | | | | | | | | | | | | | | |
| 8F-H1L1 | | | | | | | | | | | | | | | | |
| 1B-H1L1 | | | | | | | | | | | | | | | | |
| 1D-H8L1 | | | | | | | | | | | | | | | | |
| 2F-H1L3 | | | | | | | | | | | | | | | | |
| 3H-H1L1 | | | | | | | | | | | | | | | | |
| 4B-H2L1 | | | | | | | | | | | | | | | | |
| 5A-H1L1 | | | | | | | | | | | | | | | | |
| 5C-H6L1 | | | | | | | | | | | | | | | | |
| 5D-H6L2 | | | | | | | | | | | | | | | | |
| 6B-H1L2 | | | | | | | | | | | | | | | | |
| 6D-H3L1 | | | | | | | | | | | | | | | | |
| 6E-H1L1 | | | | | | | | | | | | | | | | |
| 7A-H1L1 | | | | | | | | | | | | | | | | |
| 7C-H3L1 | | | | | | | | | | | | | | | | |
| 7E-H1L1 | | | | | | | | | | | | | | | | |
| 7H-H1L1 | | | | | | | | | | | | | | | | |
| 9H-H1L1 | | | | | | | | | | | | | | | | |
| 9E-H2L2 | | | | | | | | | | | | | | | | |
| 11E-H1L1 | | | | | | | | | | | | | | | | |
| A3-3M1-B11-H1L2 | | | | | | | | | | | | | | | | |
| A3-3M1-D1-H1L3 | | | | | | | | | | | | | | | | |
| A3-3M2-D3-H2L1 | | | | | | | | | | | | | | | | |
| A3-3M2-F5-H1L2 | | | | | | | | | | | | | | | | |
| A3-3M3-C4-H3L3 | | | | | | | | | | | | | | | | |
| A3-3M3-G3-H1L1 | | | | | | | | | | | | | | | | |
| A3-2M1-A5-H1L1 | | | | | | | | | | | | | | | | |
| A3-2M2-G8-H1L2 | | | | | | | | | | | | | | | | |
| A3-2M2-H5-H3L2 | | | | | | | | | | | | | | | | |
| A3-2M3-C9-H2L1 | | | | | | | | | | | | | | | | |
| A3-2M3-E9-H1L2 | | | | | | | | | | | | | | | | |
| A3-2M3-F9-H2L1 | | | | | | | | | | | | | | | | |
| A3-2M3-G3-H2L3 | | | | | | | | | | | | | | | | |
| A3-3M3-C2-H3L1 | | | | | | | | | | | | | | | | |
| A3-2M3-C9-H2L1 | | | | | | | | | | | | | | | | |
| | | | | | | | | | A3-3M1- | A3-3M1- | A3-3M2- | A3-3M2- | A3-3M3- | A3-3M3- | A3-2M1- | A3-2M2- |
| | 6E-H1L1 | 7A-H1L1 | 7C-H3L1 | 7E-H1L1 | 7H-H1L1 | 9H-H1L1 | 9E-H2L2 | 11E-H1L1 | B11-H1L2 | D1-H1L3 | D3-H2L1 | F5-H1L2 | C4-H3L3 | G3-H1L1 | A5-H1L1 | G8-H1L2 |
| 1D-H4L1 | | | | | | | | | | | | | | | | |
| 1E-H3L1 | | | | | | | | | | | | | | | | |
| 2A-H5L4 | | | | | | | | | | | | | | | | |
| 2C-H3L2 | | | | | | | | | | | | | | | | |
| 2E-H2L2 | | | | | | | | | | | | | | | | |
| 2F-H1L1 | | | | | | | | | | | | | | | | |
| 2G-H2L1 | | | | | | | | | | | | | | | | |
| 3D-H1L2 | | | | | | | | | | | | | | | | |
| 3E-H1L2 | | | | | | | | | | | | | | | | |
| 3F-H1L2 | | | | | | | | | | | | | | | | |
| 4F-H6L1 | | | | | | | | | | | | | | | | |
| 5A-H6L1 | | | | | | | | | | | | | | | | |
| 5B-H1L1 | | | | | | | | | | | | | | | | |
| 5C-H2L1 | | | | | | | | | | | | | | | | |
| 5D-H1L2 | | | | | | | | | | | | | | | | |
| 5H-H3L1 | | | | | | | | | | | | | | | | |
| 6C-H8L1 | | | | | | | | | | | | | | | | |
| 6E-H6L1 | | | | | | | | | | | | | | | | |
| 7E-H1L1 | | | | | | | | | | | | | | | | |
| 7G-H1L1 | | | | | | | | | | | | | | | | |
| 7H-H1L1 | | | | | | | | | | | | | | | | |
| 8F-H1L1 | | | | | | | | | | | | | | | | |
| 1B-H1L1 | | | | | | | | | | | | | | | | |
| 1D-H8L1 | | | | | | | | | | | | | | | | |
| 2F-H1L3 | | | | | | | | | | | | | | | | |
| 3H-H1L1 | | | | | | | | | | | | | | | | |
| 4B-H2L1 | | | | | | | | | | | | | | | | |
| 5A-H1L1 | | | | | | | | | | | | | | | | |
| 5C-H6L1 | | | | | | | | | | | | | | | | |
| 5D-H6L2 | | | | | | | | | | | | | | | | |
| 6B-H1L2 | | | | | | | | | | | | | | | | |
| 6D-H3L1 | | | | | | | | | | | | | | | | |
| 6E-H1L1 | | | | | | | | | | | | | | | | |
| 7A-H1L1 | | | | | | | | | | | | | | | | |
| 7C-H3L1 | | | | | | | | | | | | | | | | |
| 7E-H1L1 | | | | | | | | | | | | | | | | |
| 7H-H1L1 | | | | | | | | | | | | | | | | |
| 9H-H1L1 | | | | | | | | | | | | | | | | |
| 9E-H2L2 | | | | | | | | | | | | | | | | |
| 11E-H1L1 | | | | | | | | | | | | | | | | |
| A3-3M1-B11-H1L2 | | | | | | | | | | | | | | | | |
| A3-3M1-D1-H1L3 | | | | | | | | | | | | | | | | |
| A3-3M2-D3-H2L1 | | | | | | | | | | | | | | | | |
| A3-3M2-F5-H1L2 | | | | | | | | | | | | | | | | |
| A3-3M3-C4-H3L3 | | | | | | | | | | | | | | | | |
| A3-3M3-G3-H1L1 | | | | | | | | | | | | | | | | |
| A3-2M1-A5-H1L1 | | | | | | | | | | | | | | | | |
| A3-2M2-G8-H1L2 | | | | | | | | | | | | | | | | |
| A3-2M2-H5-H3L2 | | | | | | | | | | | | | | | | |
| A3-2M3-C9-H2L1 | | | | | | | | | | | | | | | | |
| A3-2M3-E9-H1L2 | | | | | | | | | | | | | | | | |
| A3-2M3-F9-H2L1 | | | | | | | | | | | | | | | | |
| A3-2M3-G3-H2L3 | | | | | | | | | | | | | | | | |
| A3-3M3-C2-H3L1 | | | | | | | | | | | | | | | | |
| A3-2M3-C9-H2L1 | | | | | | | | | | | | | | | | |
| | | | | | | | | | | | A3-2M2- | A3-2M2- | A3-2M3- | A3-2M3- | A3-2M3- | A3-2M3- |
| | | | | | | | | | | | H5-H3L2 | C9-H2L1 | E9-H1L2 | F9-H2L1 | G3-H2L3 | [symbol] |

1D-H4L1
1E-H3L1
2A-H5L4
2C-H3L2
2E-H2L2
2F-H1L1
2G-H2L1
3D-H1L2
3E-H1L2
3F-H1L2
4F-H6L1
5A-H6L1
5B-H1L1
5C-H2L1
5D-H1L2
5H-H3L1
6C-H8L1
6E-H6L1
7E-H1L1
7G-H1L1
7H-H1L1
8F-H1L1
1B-H1L1
1D-H8L1
2F-H1L3
3H-H1L1
4B-H2L1
5A-H1L1
5C-H6L1
5D-H6L2
6B-H1L2
6D-H3L1
6E-H1L1
7A-H1L1
7C-H3L1
7E-H1L1
7H-H1L1
9H-H1L1
9E-H2L2
11E-H1L1
A3-3M1-B11-H1L2
A3-3M1-D1-H1L3
A3-3M2-D3-H2L1
A3-3M2-F5-H1L2
A3-3M3-C4-H3L3
A3-3M3-G3-H1L1
A3-2M1-A5-H1L1
A3-2M2-G8-H1L2
A3-2M2-H5-H3L2
A3-2M3-C9-H2L1
A3-2M3-E9-H1L2
A3-2M3-F9-H2L1
A3-2M3-G3-H2L3
A3-3M3-C2-H3L1
A3-2M3-C9-H2L1

[symbol]

indicates data missing or illegible when filed

### 2.4 Further Characterization of the Antibodies

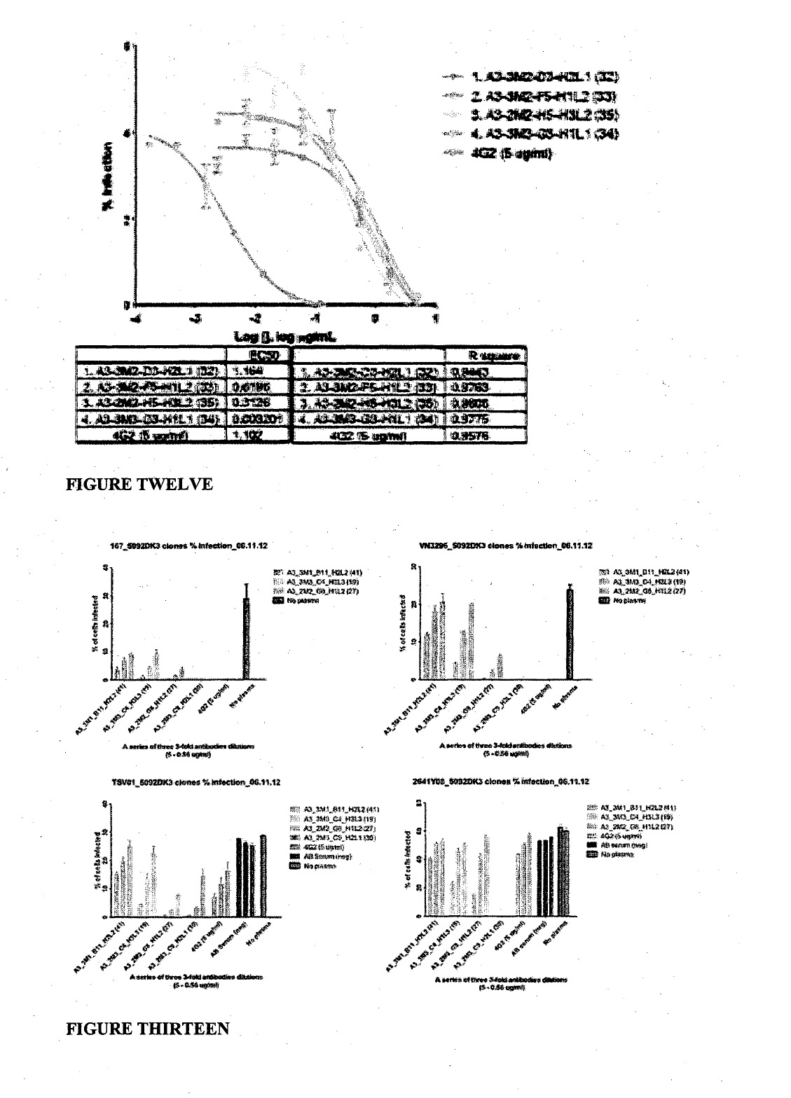

To confirm that the antibodies pertaining described herein do not bind to nonstructural protein NS1, an ELISA was performed (FIG. 10). For none of the antibodies tested binding to NS1 was observed.

### Example 3

### Additional Anti-DENV Antibodies

—Anti-DENV Antibodies Isolated from Memory B Cells

Additional antibodies were isolated from memory B cells from the two patients described in the Examples above, which were all isolated from plasmablasts.

Antibodies isolated from memory B cells from patients 10/50 that bind to dengue virus with high specificity and affinity are summarized in Table 7.

TABLE 7
binding patterns of antibodies isolated from memory B cells at day 16 after fever
onset in patient 10/50

| | Positive in UV PEG ELISA (OD450): E | | | | | | | | | |
|---|---|---|---|---|---|---|---|---|---|---|
| | Positive in Histology: H* | | | | | | | | | |
| Antibody | Den1 | Den2 | Den3 | Den4 | E protein ELISA | | | | serotype- | |
| [symbol] | (167) | (TSV01) | (VN32/96) | (2641Y08) | [symbol] | [symbol] | [symbol] | [symbol] | specificity | Epitope |
| A3-3M1-B11-H1L2 | E/H | E/H | E/H | E/H | Neg | Neg | Neg | Neg | DENV1/2/3/4 | whole virus particle |
| A3-3M1-D1-H1L3 | E | E | E | E | Neg | Neg | Neg | Neg | DENV1/2/3/4 | whole virus particle |
| A3-3M2-D3-H2L1 | E/H | E/H | E/H | E/H | Neg | Pos | Neg | Neg | DENV2 > DENV1/3/4 | E protein and whole virus particle |
| A3-3M2-F5-H1L2 | E/H | E/H | E/H | E/H | Neg | Pos | Neg | Neg | DENV2 > DENV1/3/4 | E protein and whole virus particle |
| A3-3M3-C4-H3L3 | E/H | E/H | E/H | E/H | Neg | Neg | Neg | Neg | DENV1/2 > 3/4 | whole virus particle |
| A3-3M3-G3-H1L1 | E/H | E/H | E/H | E/H | Neg | Pos | Pos | Neg | DENV1/2/3/4 | E protein and whole virus particle |
| A3-2M1-A5-H1L1 | H | H | H:nd | neg | nd | nd | nd | nd | DENV1/2/3 | Not identified yet |
| A3-2M2-G8-H1L2 | E/H | E/H | E/H | E/H | Neg | Neg | Neg | Neg | DENV1/3>2/4 | whole virus particle |
| A3-2M2-H5-H3L2 | E/H | E/H | E/H | E/H | Neg | Pos | Pos | Pos | DENV1/2/3/4 | E protein and whole virus particle |
| A3-2M3-C9-H2L1 | E/H | E/H | E/H | E/H | Neg | Neg | Neg | Neg | DENV1/2/3/4 | whole virus particle |
| A3-2M3-E9-H1L2 | E/H | E/H | E/H | E/H | nd | nd | nd | nd | DENV1/2/3/4 | Not identified yet |
| A3-2M3-F9-H2L1 | E/H | E/H | E/H | E/H | nd | nd | nd | nd | DENV1/2/3/4 | Not identified yet |
| A3-2M3-G3-H2L3 | E/H | E/H | E/H | E/H | nd | nd | nd | nd | DENV1/2/3/4 | Not identified yet |
| A3-2M3-C2-H3L1 | H | H | H | neg | nd | nd | nd | nd | DENV1/2/3 | Not identified yet |

nd: not done
*Histology on infected BHK21 cells as described in EXAMPLE 2 above [symbol]
indicates data missing or illegible when filed

### —Neutralizing Data for E Protein Binding Antibodies

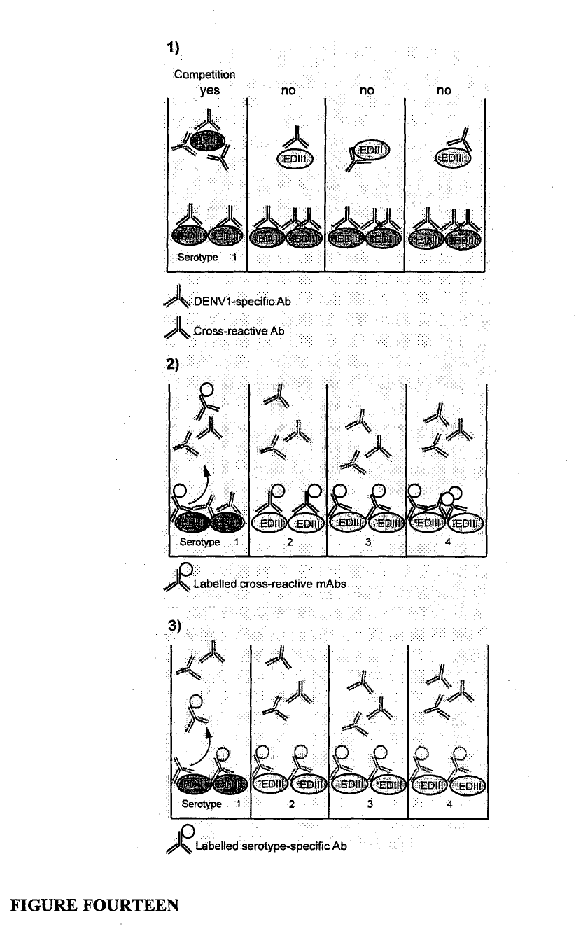

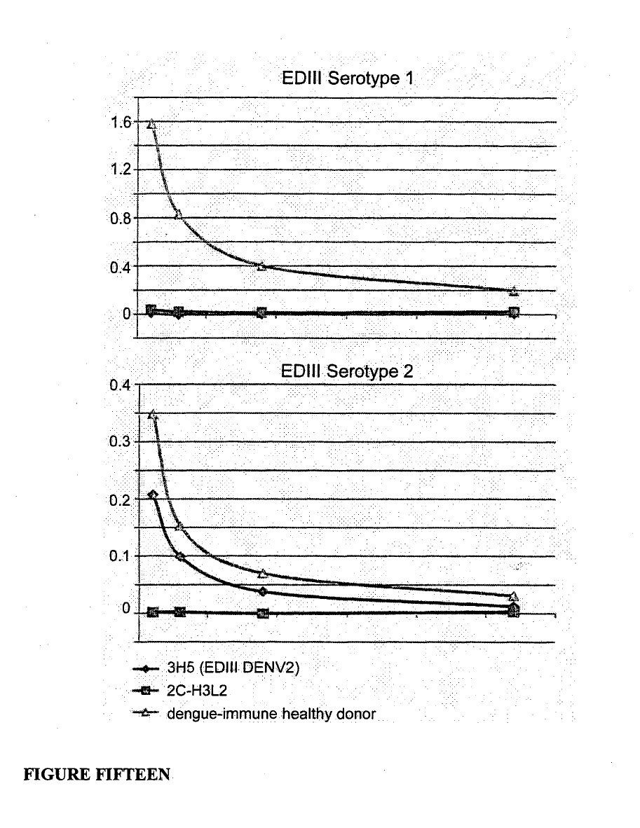

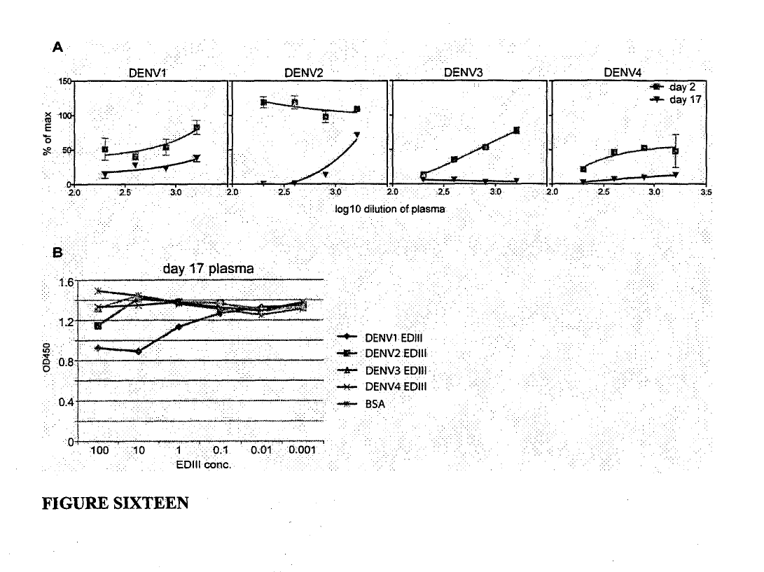

Since patient 10/50 had previous DENV-2 and DENV-3 infections we tested the capacity of the antibodies specific for E protein to neutralize these two virus serotypes. The antibodies were tested at a concentration range of 5 to 0.002 ug/ml. 4G2 was used as a positive control antibody. Serum of blood type AB (commercially available) was used as a negative control (FIGS. 11, 12 and 13).

### Example 4

### Exemplary Assays for Detection of Dengue Serotype-Specific Antibodies

### —Exemplary Competition ELISA Approaches:

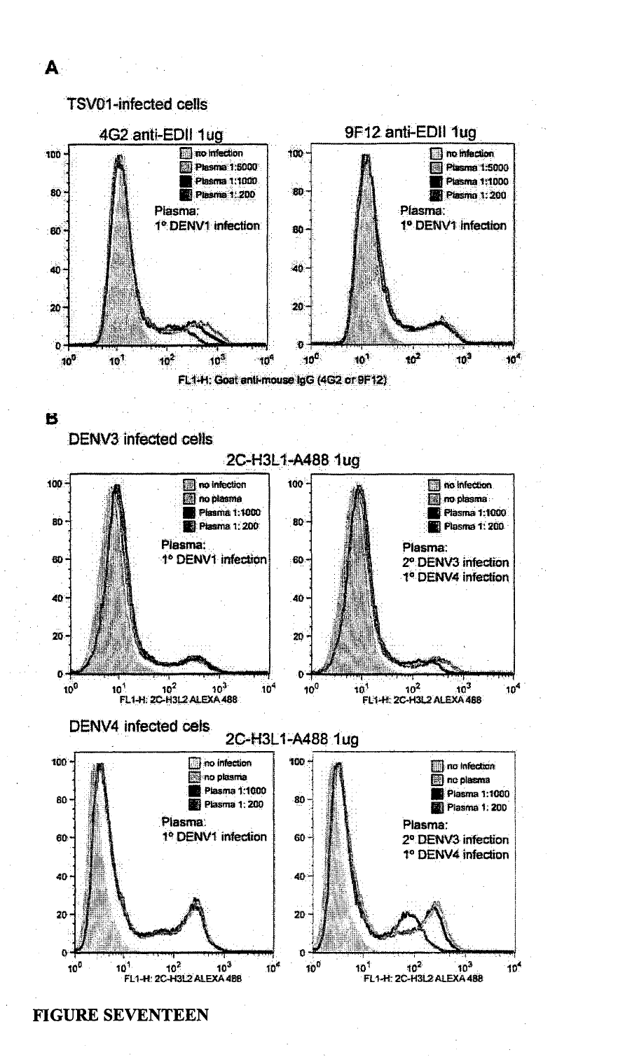

FIG. 14 details three exemplary competition. ELISA approaches employing antibodies as described herein.

### (i) Approach One:

Competition with EDIII proteins of different serotypes in an ELISA-based system to differentiate between cross-serotype and serotype-specific antibodies. Only EDIII protein of the correct serotype in solution will compete with the immobilized EDIII protein for serotype-specific antibodies, resulting in reduced ELISA signal when the latter are released from the immobilized EDIII.

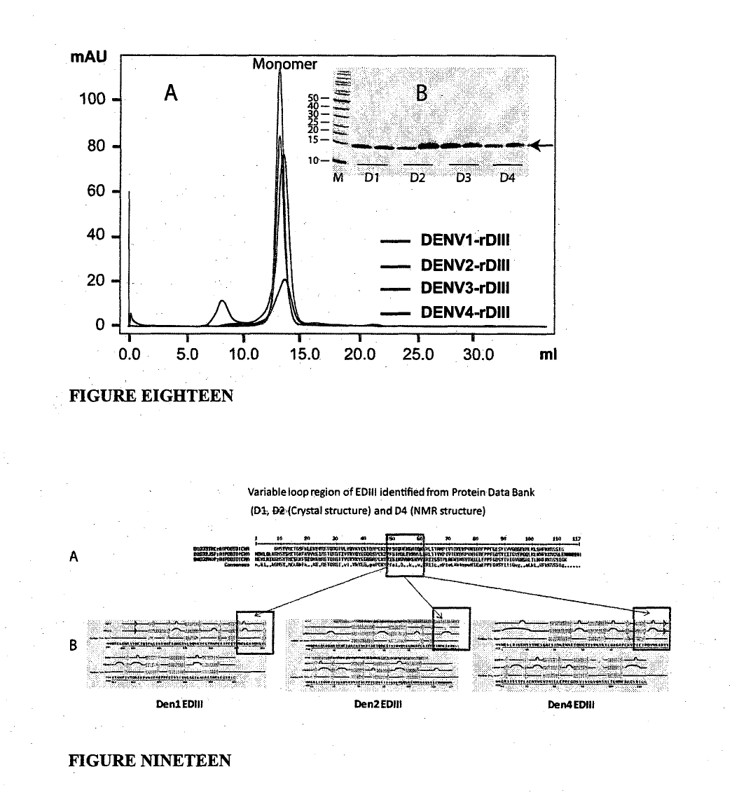

EDIII protein contains neutralizing domain(s) and is thus prone to mutate to escape antibody-mediated immune responses. While the EDIII is the least conserved protein of the E protein, parts of it are still conserved, and EDIII-specific antibodies can be cross-reactive. An EDIII-ELISA is thus not useful to determine the serotype (FIG. 15).

However, when adding an increasing amount of soluble EDIII protein the competition between serotypes will allow to determine the serotype of the previous infection. Once an equilibrium is reached, more serotype-specific EDIII-specific antibodies will have bound to an excess of soluble EDIII protein of the respective serotypes, whereas cross-reactive EDIII-specific antibodies will bind equally to both bound and soluble EDIII protein. The loss of antibodies bound to EDIII protein on the plate is detected as decreasing fluorescent signal.

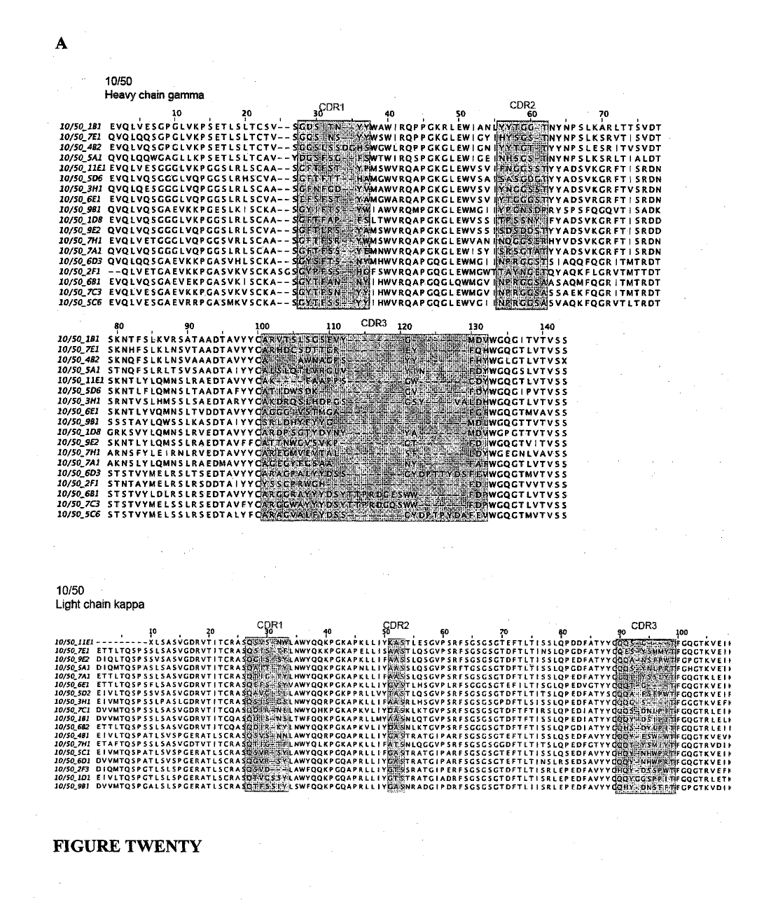

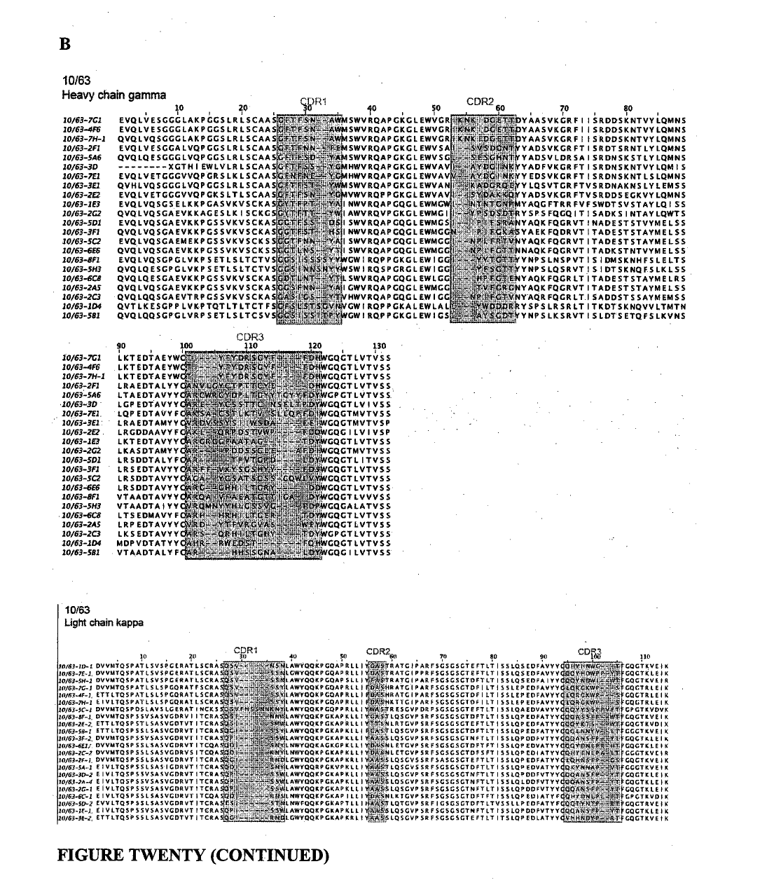

FIG. 16 shows the neutralization assay and EDIII protein competition assay with the plasma of a patient with secondary DENV2 infection. The neutralization assay performed with plasma from two days after fever onset suggested, previous DENV1 or 3 infections, whereas the competition ELISA done at early convalescence clearly indicated a previous DENV1 infection.

### (ii) Approach Two:

The immobilized EDIII or E proteins are first tagged with labeled cross-reactive human monoclonal antibodies. Upon addition of the patient blood sample, the high affinity serotype-specific serum-antibodies will compete with the bound labeled monoclonal antibodies, resulting in their release, which is subsequently measured (e.g. fluorescence intensity). This test is also based on the fact that cross-reactive antibodies are of lower affinity than serotype-specific ones.

Disclosed herein are a large set of antibodies from patients experiencing acute dengue infection. Many of these antibodies are cross-reactive, and when used in a competition assay, they should be replaced by higher affinity serotype-specific antibodies present in a given patient sample. Variable regions mAb sequences are shown in FIG. 20.

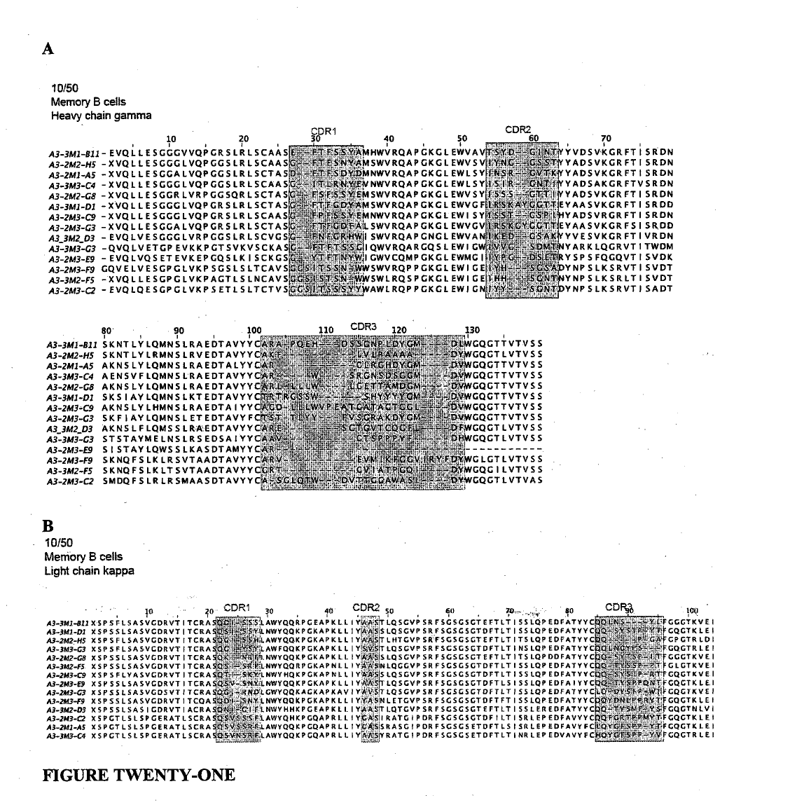

FIG. 17a shows that convalescent patient plasma can replace a cross-reactive EDII-specific 4G2, but not EDIII-specific 9F12. When using patient plasma taken during early convalescence when most antibodies are cross-reactive, an equally cross-reactive antibody 2C-H3L2 is not replaced. However, a plasma from a secondary infection patient containing antibodies with higher affinity from the first infection is able to replace 2C-H3L2 (FIG. 17b).

This approach has been established using flow cytometry, which is not as easily applicable as ELISA. It is therefore suitable to adopt this technology to ELISA.

Since cross-reactive antibodies can still have a higher affinity for one serotype it is necessary to use a combination of several cross-reactive antibodies to coat the E protein or EDIII in Approach 2. This will increase the chance that a detectable amount of labeled antibodies will be released when high affinity antibodies replace them.

(iii) Approach Three:

The immobilized EDIII or E proteins are first tagged with labeled artificially engineered, serotype-specific antibody (one per serotype). These antibodies are engineered against EDIII surface motifs, especially the surface loops that are unique in sequence to each serotype and that are immunogenic against which the antibody responses are known to be elicited from infected patients. Upon the addition of the patient blood sample, the serotype-specific serum-antibodies will compete with the labeled artificial monoclonal antibodies for the binding to the selected surface motif of the EDIII protein, resulting in their release which is subsequently measured (e.g. fluorescence intensity).

Purified serotype-specific loop regions (see below) can be used to isolate serotype-specific antibodies. The serotype specific artificial antibody will be isolated from a naive antibody phage display library. The 3×10¹⁰ diversity library was constructed using a variety of lymphocyte tissues from a large number of donors of diverse ethnic groups. To select for the antibodies against the EDIII of a specific serotype, the recombinant target protein will be biotinylated. The target EDIII is then incubated with the antibody library in the presence of the non-biotinylated EDIII of the 3 other serotypes at large excess (>20-fold over the biotinylated target). Using this strategy, the cross-reactive antibodies will preferentially be driven to binding to the non-biotinylated proteins, allowing the pre-determined serotype specific antibodies to be isolated by magnetic streptavidin beads. The thus isolated antibody phage will be amplified in E. coli culture for the next round of selection. Three to five rounds of selection with increasing stringency will be carried out. Following the selection process, individual antibodies will be expressed, purified, and examined for their affinity and selectivity by ELISA. Antibodies with the highest serotype-differentiating capacity will be evaluated for diagnostic applications.

### —Expression and Purification of Viral Proteins

In order to obtain purified DIII proteins for the above-mentioned studies, DENV1 to DENV4 envelope DIII proteins are expressed, purified and characterized. Recombinant DENV1 to DENV4 envelope DIII protein (r-DIII) are purified from inclusion bodies in E. coli.

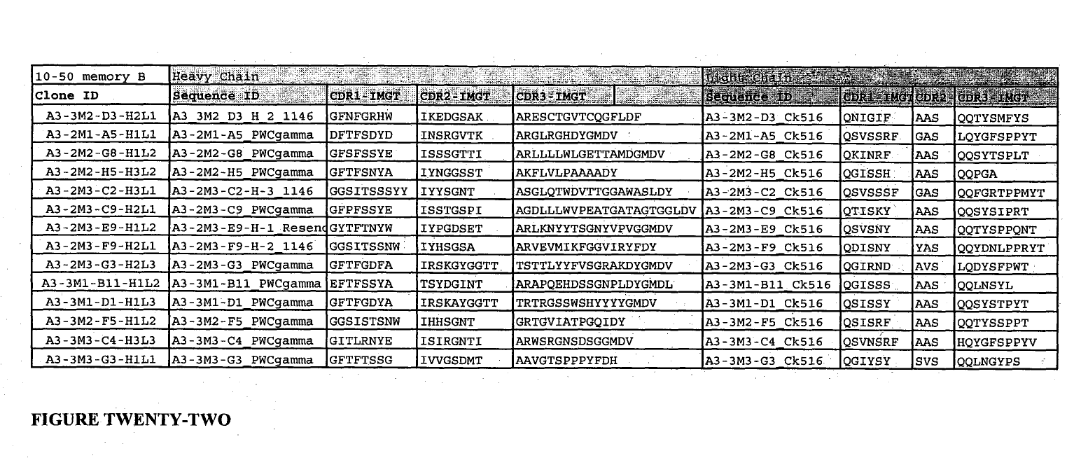

Recombinant proteins corresponding to the EDIII of DENV1 to 4 were expressed in bacterial system and the expressed proteins were extracted from inclusion bodies. The extracted inclusion body was subjected to initial Ni-NTA purification followed by refolding of the protein by slow dialysis. After the completion of the dialysis the protein was subjected to FPLC purification to obtain highly purified EDIII proteins which will be further used for the ELISA assays platforms in this proposal. FIG. 18 shows the FPLC profiles and a gel showing bands corresponding to the EDIII of DENV 1 to 4. The purification procedure is well-established in the laboratory and reproducible for the production of milligram amount of proteins.

The methodology to identify the variable loop regions is shown below.

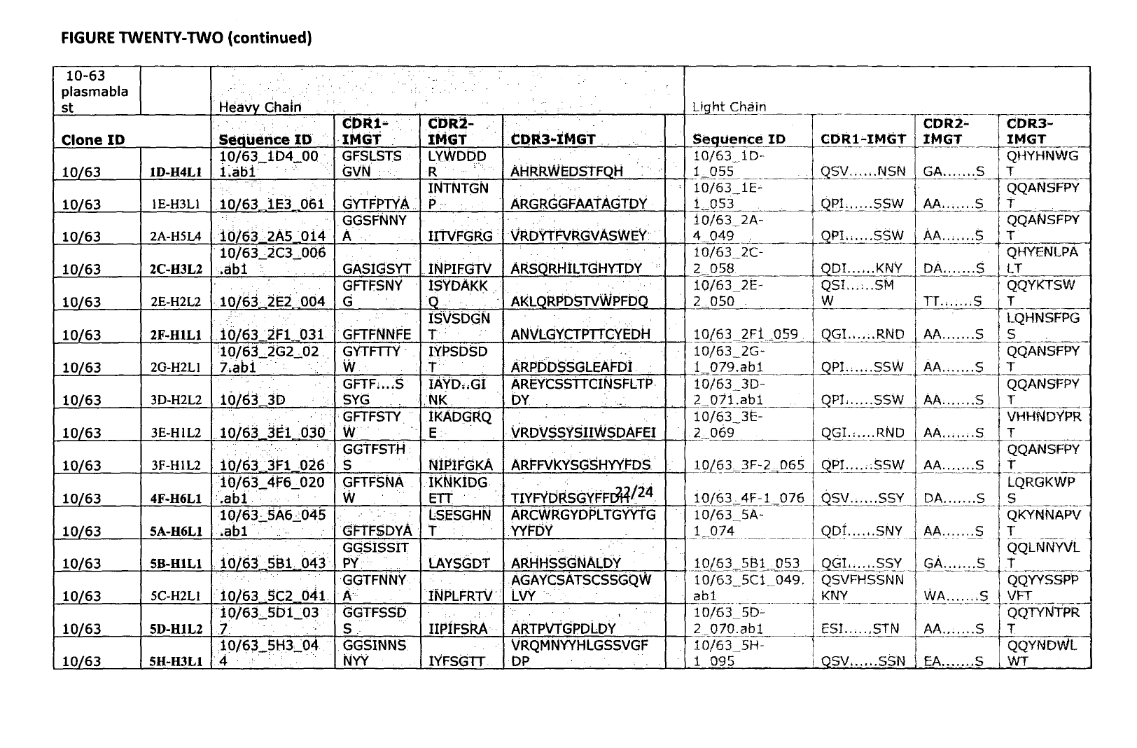

This approach uses the available protein sequences of EDIII proteins, followed by an extensive bioinformatics analyses to identify the variable loop regions of the respective DENV serotypes. By this we can identify new epitopes for EDIII protein. Further validation was done to confirm whether region lies in the loop region or sub domain of EDIII protein with the available crystal structures (DENV 1 and 2) or NMR (DENV 4) data. There is no crystal structure or NMR data for DENV3. Preliminary analysis identified the variable regions for the respective DENV EDIII proteins (FIG. 19). The identified variable regions can be synthesized using solid phase peptide synthesis or could be expressed in bacterial systems followed by purification. They will be used to generate antibodies which specifically recognise a particular serotype.

### REFERENCES

Dowd K A, Jost C A, Durbin A P, Whitehead S S, Pierson T C. A dynamic landscape for antibody binding modulates antibody-mediated neutralization of west nile virus. PLoS Pathog. 2011; 7(6):e1002111. Epub 2011/07/09. doi: 10.1371/journal.ppat.1002111 PPATHOGENS-D-10-00370 [pii]. PubMed PMID: 21738473; PubMed Central PMCID: PMC3128118.
Durocher, Y., S. Perret, and A. Kamen. 2002. High-level and high-throughput recombinant protein production by transient transfection of suspension-growing human 293-EBNA1 cells. Nucleic Acids Res 30:E9.
Giudicelli, V., P. Duroux, C. Ginestoux, G. Folch, J. Jabado-Michaloud, D. Chaume, and M. P. Lefranc. 2006. IMGT/LIGM-DB, the IMGT comprehensive database of immunoglobulin and T cell receptor nucleotide sequences. Nucleic Acids Res 34:D781-784.
Kontermann R. Dual targeting strategies with bispecific antibodies. MAbs. 2012; 4(2):182-97. Epub March 1.
Kraus, A. A., W. Messer, L. B. Haymore, and A. M. de Silva. 2007. Comparison of plaque- and flow cytometry-based methods for measuring dengue virus neutralization. J Clin Microbiol 45:3777-3780.
Low, J. G., E. E. Ooi, T. Tolfvenstam, Y. S. Leo, M. L. Hibberd, L. C. Ng, Y. L. Lai, G. S. Yap, C. S. Li, S. G. Vasudevan, and A. Ong. 2006. Early Dengue infection and outcome study (EDEN)—study design and preliminary findings. Ann Acad Med Singapore 35:783-789.
McBride, W., and S. Vasudevan. 1995. Relationship of a dengue 2 isolate from Townsville, 1993, to international isolates. Comm Dis Intelligence 19:522-523. Organization, W.H. 2009. Dengue: guidelines for diagnosis, treatment, prevention and control. In.
Sabchareon A, Wallace D, Sirivichayakul C, Limkittikul K, Chanthavanich P, Suvannadabba S, et al. Protective efficacy of the recombinant, live-attenuated, CYD tetravalent dengue vaccine in That schoolchildren: a randomised, controlled phase 2b trial. Lancet. 2012. Epub 2012/09/15. doi: 10.1016/S0140-6736(12)61428-7. PubMed PMID: 22975340.
Schreiber, M. J., E. C. Holmes, S. H. Ong, H. S. Soh, W. Liu, L. Tanner, P. P. Aw, H. C. Tan, L. C. Ng, Y. S. Leo, J. G. Low, A. Ong, E. E. Ooi, S. G. Vasudevan, and M. L. Hibberd. 2009. Genomic epidemiology of a dengue virus epidemic in urban Singapore. J Virol 83:4163-4173.
Schul, W., W. Liu, H. Y. Xu, M. Flamand, and S. G. Vasudevan. 2007. A dengue fever viremia model in mice shows reduction in viral replication and suppression of the inflammatory response after treatment with antiviral drugs. J Infect Dis 195:665-674.
Smith, K., L. Garman, J. Wrammert, N. Y. Zheng, J. D. Capra, R. Ahmed, and P. C. Wilson. 2009. Rapid generation of fully human monoclonal antibodies specific to a vaccinating antigen. Nat Protoc 4:372-384.
Souto-Carneiro, M. M., N. S. Longo, D. E. Russ, H. W. Sun, and P. E. Lipsky. 2004. Characterization of the human Ig heavy chain antigen binding complementarity determining region 3 using a newly developed software algorithm, JOINSOLVER. J Immunol 172:6790-6802.
Teoh E P, Kukkaro P, Teo E W, Lim A P, Tan T T, Yip A, et al. The structural basis for serotype-specific neutralization of dengue virus by a human antibody. Sci Transl Med. 2012; 4(139):139ra83. Epub 2012/06/23. doi: 10.1126/scitranslmed.3003888. PubMed PMID: 22723463.
Thompson B S, Moesker B, Smit J M, Wilschut J, Diamond M S, Fremont D H. A therapeutic antibody against west nile virus neutralizes infection by blocking fusion within endosomes. PLoS Pathog. 2009; 5(5):e1000453. Epub 2009/05/30. doi: 10.1371/journal.ppat.1000453. PubMed PMID: 19478866; PubMed Central PMCID: PMC2679195.
Umashankar, M., C. Sanchez-San Martin, M. Liao, B. Reilly, A. Guo, G. Taylor, and M. Kielian. 2008. Differential cholesterol binding by class II fusion proteins determines membrane fusion properties. J Virol 82:9245-9253.
Warter, L., C. Y. Lee, R. Thiagarajan, M. Grandadam, S. Lebecque, R. T. Lin, S. Bertin-Maghit, L. F. Ng, J. P. Abastado, P. Despres, C. I. Wang, and A. Nardin. 2011. Chikungunya virus envelope-specific human monoclonal antibodies with broad neutralization potency. J Immunol 186:3258-3264
WHO. Dengue: guidelines for diagnosis, treatment, prevention and control. 2009:
Wrammert, J., K. Smith, J. Miller, W. A. Langley, K. Kokko, C. Larsen, N. Y. Zheng, I. Mays, L. Garman, C. Helms, J. James, G. M. Air, J. D. Capra, R. Ahmed, and P. C. Wilson. 2008. Rapid cloning of high-affinity human monoclonal antibodies against influenza virus. Nature 453:667-671.
Wrammert, J., N. Onlamoon, R. S. Akondy, G. C. Perng, K. Polsrila, A. Chandele, M. Kwissa, B. Pulendran, P. C. Wilson, O. Wittawatmongkol, S. Yoksan, N. Angkasekwinai, K. Pattanapanyasat, K. Chokephaibulkit, and R. Ahmed. 2012. Rapid and massive virus-specific plasmablast responses during acute dengue virus infection in humans. J Virol 86:2911-2918.

1. A human monoclonal antibody capable of specifically binding to an envelope (E) protein of at least one dengue virus serotype, wherein the antibody comprises a light chain variable domain sequence comprising the first, second, and third complementarity determining region (CDR) sequences of a single clone designated in FIG. 20, 21 or 22.

2. A cross-reactive human monoclonal antibody derived from a memory B lymphocyte capable of specifically binding to an E protein of at least one dengue virus serotype, wherein the antibody comprises all CDR sequences from one pair of the following heavy and light chain variable domain peptide sequences: SEQ ID NOs: 189 and 190; SEQ ID NOs: 191 and 192; SEQ ID NOs: 199 and 200; SEQ ID NOs: 201 and 202: SEQ ID NOs: 203 and 204; SEQ ID NOs: 207 and 208; SEQ ID NOs: 209 and 210; SEQ ID NOs: 211 and 212; SEQ ID NOs: 213 and 214; or SEQ ID NOs: 215 and 216.

3. A cross-reactive human monoclonal antibody derived from a plasmablast capable of specifically binding to an E protein of at least one dengue virus serotype, wherein the antibody comprises all CDR sequences from one pair of the following heavy and light chain variable domain peptide sequences: SEQ ID NOs: 113 and 114; SEQ ID NOs: 115 and 116; SEQ ID NOs: 121 and 122; SEQ ID NOs: 123 and 124; SEQ ID NOs: 127 and 128; SEQ ID NOs: 129 and 130; SEQ ID NOs: 131 and 132; SEQ ID NOs: 133 and 134; SEQ ID NOs: 135 and 136; SEQ ID NOs: 137 and 138; SEQ ID NOs: 139 and 140; SEQ ID NOs: 141 and 142; SEQ ID NOs: 143 and 144; SEQ ID NOs: 145 and 146; SEQ ID NOs: 147 and 148; SEQ ID NOs: 151 and 152; SEQ ID NOs: 153 and 154; SEQ ID NOs: 155 and 156; SEQ ID NOs: 157 and 158; SEQ ID NOs: 159 and 160; SEQ ID NOs: 161 and 162; SEQ ID NOs: 163 and 164; SEQ ID NOs: 165 and 166; SEQ ID NOs: 169 and 170; SEQ ID NOs: 171 and 172; SEQ ID NOs: 173 and 174; SEQ ID NOs: 177 and 178; SEQ ID NOs: 179 and 180; SEQ ID NOs: 181 and 182; SEQ ID NOs: 183 and 184; SEQ ID NOs: 187 and 188; SEQ ID NOs: 189 and 190; SEQ ID NOs: 191 and 192; SEQ ID NOs: 193 and 104: SEQ ID NOs: 195 and 196; SEQ ID NOs: 197 and 198; SEQ ID NOs: 199 and 200; SEQ ID NOs: 201 and 202; SEQ ID NOs: 203 and 204; SEQ ID NOs: 205 and 206; SEQ ID NOs: 207 and 208; SEQ ID NOs: 209 and 210; SEQ ID NOs: 211 and 212; SEQ ID NOs: 213 and 214; or SEQ ID NOs: 215 and 216.

4. A serotype-specific human monoclonal antibody derived from a plasmablast capable of specifically binding to an E protein of one or two dengue virus serotypes with higher affinity compared to E proteins of other dengue virus serotypes, wherein the antibody comprises all CDR sequences from one pair of the following heavy and light chain variable domain peptide sequences: SEQ ID NO: 109 and SEQ ID NO: 110; SEQ ID NO: 111 and SEQ ID: NO 112; SEQ ID NO: 119 and SEQ ID NO: 120: SEQ ID NO: 125 and SEQ ID NO: 126; SEQ ID NO: 149 and SEQ ID NO: 150; SEQ ID NO: 153 and SEQ ID NO: 154; SEQ ID NO: 167 and SEQ ID NO: 168; SEQ ID NO: 175 and SEQ ID NO: 176; SEQ ID NO: 185 or SEQ ID NO: 186.

5. A serotype-specific human monoclonal antibody derived from a memory B lymphocyte capable of specifically binding to an E protein of one or two dengue virus serotypes with higher affinity compared to E proteins of other dengue virus serotypes, wherein the antibody comprises all CDR sequences from one pair of the following heavy and light chain variable domain peptide sequences: SEQ ID NO: 193 and SEQ ID NO: 194; SEQ ID NO: 195 and SEQ ID NO: 196; SEQ ID NO: 197 or 198; or SEQ ID NO: 205 and SEQ ID NO: 206.

6. The monoclonal antibody of claim 1, wherein the antibody comprises full heavy and light chain variable domain sequences from said pair.

7. A bi-specific antibody comprising a variable domain of a first monoclonal antibody linked to a variable domain of a second monoclonal antibody, wherein the first and second antibody each comprise all CDR sequences from one pair of heavy and light chain variable domain peptide sequences according to claim 1.

8. A bispecific antibody comprising a variable domain of a first monoclonal antibody linked to a variable domain of a second monoclonal antibody, wherein first and second antibodies are a combination as defined in Table 5 or Table 6.

9. A pharmaceutical composition or kit comprising the antibody of claim 1.

10. An isolated polynucleotide encoding first, second, and third complementarity determining region (CDR) sequences in the heavy chain variable domain of a single clone designated in FIG. 20, 21 or 22.

11. An isolated polynucleotide encoding first, second, and third complementarity determining region (CDR) sequences in the light chain heavy chain variable domain of a single clone designated in FIG. 20, 21 or 22.

12. An isolated polynucleotide encoding an antibody according to claim 1.

13.-15. (canceled)

16. An antibody according to claim 1 for use in the prevention or treatment of dengue virus infection.

17. The antibody according to claim 16, wherein the antibody comprises:
all CDR sequences from any one or more of the following pairs of heavy and light chain variable domain peptide sequences (SEQ ID NOs: 109 and 110; SEQ ID NOs: 111 and 112; SEQ ID NOs: 117 and 118; SEQ ID NOs: 119 and 120; SEQ ID NOs: 125 and 126; SEQ ID NOs: 149 and 150; SEQ ID NOs: 153 and 154; SEQ ID NOs: 167 and 168; SEQ ID NOs: 175 and 176; SEQ ID NOs: 185 and 186; SEQ ID NOs: 189 and 190; SEQ ID NOs: 191 and 192; SEQ ID NOs: 193 and 194; SEQ ID NOs: 195 and 196; SEQ ID NOs: 197 and 198; SEQ ID NOs: 199 and 200; SEQ ID NOs: 201 and 202: SEQ ID NOs: 203 and 204; SEQ ID NOs: 205 and 206; SEQ ID NOs: 207 and 208; SEQ ID NOs: 209 and 210; SEQ ID NOs: 211 and 212; SEQ ID NOs: 213 and 214; SEQ ID NOs: 215 and 216; or
a series of heavy and light chain variable domain CDR sequences in accordance with any one clone as designated in FIG. 22 herein.

18. (canceled)

19. A method for detecting immunity to dengue virus in a subject, comprising:
coating at least one insoluble support with a dengue virus E protein,
contacting the E protein coated to the support with a first antibody comprising:
an antibody according to claim 1; and
the biological sample of the subject;
and detecting a presence or absence of competitive binding to the E protein between the first antibody and an antibody specific to the E protein that may be present in the sample,
wherein detection of said competitive binding indicates pre-existing immunity to dengue virus in the subject.

20. The method according to claim 19, comprising:
coating four of said insoluble supports with E protein, wherein each said support is coated with E protein from a distinct dengue virus serotype, and each said support is isolated from all other said supports;
contacting the E protein coated to each said isolated insoluble support with said first antibody and said biological sample; and
determining the amount of first antibody bound to the E protein coated on each said isolated insoluble support;
wherein detection of less first antibody bound to the E protein coated on one of said supports when compared to at least one other of said supports indicates pre-existing immunity to dengue virus in the subject,
and wherein said pre-existing immunity is specific to the dengue serotype of the E protein to which less first antibody is bound.

21. The method according to claim 20, comprising:
coating four of said insoluble supports with E protein, wherein each said support is coated with E protein from the same dengue virus serotype, and each said support is isolated from all other said supports;
contacting the E protein coated to each said isolated insoluble support with said first antibody, said biological sample and soluble E protein of a specific dengue virus serotype, wherein each said isolated insoluble support is contacted with soluble E protein from a different dengue virus serotype; and
determining the amount of first antibody bound to the E protein of each said isolated insoluble support;
wherein detection of less first antibody bound to the E protein coated on one of said supports when compared to at least one other of said supports indicates pre-existing immunity to dengue virus in the subject,
and wherein said pre-existing immunity is specific to the dengue serotype of the soluble E protein contacted with the E protein to which less first antibody is bound.

22. A method for detecting immunity to dengue virus in a subject, comprising:
coating four insoluble supports with E protein, wherein each said support is coated with E protein from a distinct dengue virus serotype, and each said support is isolated from all other said supports;
contacting the E protein coated on each said isolated support with a biological sample from the subject, and, a first antibody according to claim 1;
wherein the first antibody contacted with the E protein coated on each said isolated insoluble support binds specifically to that said E protein, and cannot bind to an E protein coated to any other of said supports;
determining the amount of first antibody bound to the E protein of each said isolated insoluble support;
wherein detection of less first antibody bound to the E protein coated on one of said supports when compared to at least one other of said supports indicates pre-existing immunity to dengue virus in the subject,
and wherein said pre-existing immunity is specific to the dengue serotype of the E protein to which less first antibody is bound.

23. The method according to claim 22, wherein said first antibody is an antibody derived from a plasmablast capable of specifically binding to an E protein of one or two dengue virus serotypes with higher affinity compared to E proteins of other dengue virus serotypes, wherein the antibody comprises all CDR sequences from one pair of the following heavy and light chain variable domain peptide sequences: SEQ ID NO: 109 and SEQ ID NO: 110; SEQ ID NO: 111 and SEQ ID: NO 112; SEQ ID NO: 119 and SEQ ID NO: 120: SEQ ID NO: 125 and SEQ ID NO: 126; SEQ ID NO: 149 and SEQ ID NO: 150; SEQ ID NO: 153 and SEQ ID NO: 154; SEQ ID NO: 167 and SEQ ID NO: 168; SEQ ID NO: 175 and SEQ ID NO: 176; SEQ ID NO: 185 or SEQ ID NO: 186.

24. The method according to claim 19, wherein any one or more of said insoluble supports is a bead or a well in culture plate.

25. The method according to claim 19, wherein the first antibody is labelled with a detectable marker.

26. A method for detecting immunity to dengue virus in a subject, comprising:
coating at least one insoluble support with a first antibody comprising all heavy and light chain variable domain CDR sequences from an antibody according to claim 1;
contacting the first antibody coated on the support with dengue virus particles to thereby allow said virus particles to bind to said first antibody;
contacting the virus particles with a biological sample from the subject to thereby allow any dengue virus-specific antibodies that may be present in said sample to bind to said virus particles; and
detecting whether said virus particles are bound by any said dengue virus-specific antibodies to thereby determine whether immunity to dengue virus exists in the subject.

27. The method according to claim 26, wherein the first antibody is only capable of binding to an E protein from one dengue virus serotype.

28. The method according to claim 19, wherein the E protein is E domain III (EDIII) protein.
